SEQUENCE LISTING (171 sequences)

SEQ ID NO: 1 - protein, 16 aa, Artificial Sequence
  modified peptide derived from HSA Sequence
LVRYTKKVPQVSTPTL

SEQ ID NO: 2 - protein, 12 aa, Artificial Sequence
  modified peptide derived from HSA Sequence
IVRWSKKVPCVS

SEQ ID NO: 3 - protein, 12 aa, Artificial Sequence
  modified peptide derived from HSA Sequence
ILRWSKKVPCVS

SEQ ID NO: 4 - protein, 12 aa, Artificial Sequence
  modified peptide derived from HSA Sequence
IFRWSKKVPCVS

SEQ ID NO: 5 - protein, 12 aa, Artificial Sequence
  modified peptide derived from HSA Sequence
IVRWSRKVPCVS

SEQ ID NO: 6 - protein, 12 aa, Artificial Sequence
  modified peptide derived from HSA Sequence
IVRWSHKVPCVS

SEQ ID NO: 7 - protein, 12 aa, Artificial Sequence
  modified peptide derived from HSA Sequence
IVRWSKKLPCVS

SEQ ID NO: 8 - protein, 12 aa, Artificial Sequence
  modified peptide derived from HSA Sequence
IVRWSKKIPCVS

SEQ ID NO: 9 - protein, 12 aa, Artificial Sequence
  modified peptide derived from HSA Sequence
IVRWSKKFPCVS

SEQ ID NO: 10 - protein, 12 aa, Artificial Sequence
  modified peptide derived from HSA Sequence
ILRWSRKVPCVS

SEQ ID NO: 11 - protein, 12 aa, Artificial Sequence
  modified peptide derived from HSA Sequence
ILRWSHKVPCVS

SEQ ID NO: 12 - protein, 12 aa, Artificial Sequence
  modified peptide derived from HSA Sequence
IFRWSRKVPCVS

SEQ ID NO: 13 - protein, 12 aa, Artificial Sequence
  modified peptide derived from HSA Sequence
IFRWSHKVPCVS

SEQ ID NO: 14 - protein, 12 aa, Artificial Sequence
  modified peptide derived from HSA Sequence
IVRWSKKMPCVS

SEQ ID NO: 15 - protein, 12 aa, Artificial Sequence
  modified peptide derived from HSA Sequence
ILRWSRKMPCVS

SEQ ID NO: 16 - protein, 12 aa, Artificial Sequence
  modified peptide derived from HSA Sequence
  NON_STD 11: Xaa = D-Val
IVRWSKKVPCXS

SEQ ID NO: 17 - protein, 12 aa, Artificial Sequence
  modified peptide derived from HSA Sequence
  NON_STD 11: Xaa = D-Val
ILRWSRKVPCXS

SEQ ID NO: 18 - protein, 12 aa, Artificial Sequence
  modified peptide derived from HSA Sequence
IIRWSRKMPCVS

SEQ ID NO: 19 - protein, 12 aa, Artificial Sequence
  modified peptide derived from HSA Sequence
IMRWSRKMPCVS

SEQ ID NO: 20 - protein, 12 aa, Artificial Sequence
  modified peptide derived from HSA Sequence
ILRWSRKMPCLS

SEQ ID NO: 21 - protein, 12 aa, Artificial Sequence
  modified peptide derived from HSA Sequence
ILRWSRKMPCFS

SEQ ID NO: 22 - protein, 12 aa, Artificial Sequence
  modified peptide derived from HSA Sequence
ILRWSRKMPCMS

SEQ ID NO: 23 - protein, 12 aa, Artificial Sequence
  modified peptide derived from HSA Sequence
ILRWSRKLPCVS

SEQ ID NO: 24 - protein, 12 aa, Artificial Sequence
  modified peptide derived from HSA Sequence
ILRWSRKFPCVS

SEQ ID NO: 25 - protein, 12 aa, Artificial Sequence
  modified peptide derived from HSA Sequence
ILRWSRKVPSVS

SEQ ID NO: 26 - protein, 12 aa, Artificial Sequence
  modified peptide derived from HSA Sequence
ILRWSRKMPSVS

SEQ ID NO: 27 - protein, 12 aa, Artificial Sequence
  MOD_RES 1: ACETYLATION
  modified peptide derived from HSA Sequence
SLRWSRKMPCVS

SEQ ID NO: 28 - protein, 12 aa, Artificial Sequence
  NON_STD 1: Xaa = acetylated D-Ser
  modified peptide derived from HSA Sequence
XLRWSRKMPCVS

SEQ ID NO: 29 - protein, 12 aa, Artificial Sequence
  modified peptide derived from HSA Sequence
MLRWSRKMPCVS

SEQ ID NO: 30 - protein, 12 aa, Artificial Sequence
  NON_STD 1: Xaa = D-Met
  modified peptide derived from HSA Sequence
XLRWSRKMPCVS

SEQ ID NO: 31 - protein, 12 aa, Artificial Sequence
  NON_STD 1: Xaa = D-Leu
  modified peptide derived from HSA Sequence
XLRWSRKMPCVS

SEQ ID NO: 32 - protein, 12 aa, Artificial Sequence
  NON_STD 1: Xaa = D-Phe
  modified peptide derived from HSA Sequence
XLRWSRKMPCVS

SEQ ID NO: 33 - protein, 12 aa, Artificial Sequence
  modified peptide derived from HSA Sequence
GLRWSRKMPCVS

SEQ ID NO: 34 - protein, 12 aa, Artificial Sequence
  MOD_RES 1: ACETYLATION
  modified peptide derived from HSA Sequence
SMRWSRKMPCVS

SEQ ID NO: 35 - protein, 12 aa, Artificial Sequence
  NON_STD 1: Xaa = acetylated D-Ser
  modified peptide derived from HSA Sequence
XMRWSRKMPCVS

SEQ ID NO: 36 - protein, 12 aa, Artificial Sequence
  modified peptide derived from HSA Sequence
MMRWSRKMPCVS

SEQ ID NO: 37 - protein, 12 aa, Artificial Sequence
  NON_STD 1: Xaa = D-Met
  modified peptide derived from HSA Sequence
XMRWSRKMPCVS

SEQ ID NO: 38 - protein, 12 aa, Artificial Sequence
  NON_STD 1: Xaa = D-Leu
  modified peptide derived from HSA Sequence
XMRWSRKMPCVS

SEQ ID NO: 39 - protein, 12 aa, Artificial Sequence
  NON_STD 1: Xaa = D-Phe
  modified peptide derived from HSA Sequence
XMRWSRKMPCVS

SEQ ID NO: 40 - protein, 12 aa, Artificial Sequence
  NON_STD 1: Xaa = D-Gly
  modified peptide derived from HSA Sequence
XMRWSRKMPCVS

SEQ ID NO: 41 - protein, 12 aa, Artificial Sequence
  modified peptide derived from HSA Sequence
MLRWSRKLPCVS

SEQ ID NO: 42 - protein, 12 aa, Artificial Sequence
  NON_STD 1: Xaa = D-Met
  modified peptide derived from HSA Sequence
XLRWSRKLPCVS

SEQ ID NO: 43 - protein, 12 aa, Artificial Sequence
  modified peptide derived from HSA Sequence
  NON_STD 2: Xaa = D-Leu
IXRWSRKLPCVS

SEQ ID NO: 44 - protein, 12 aa, Artificial Sequence
  modified peptide derived from HSA Sequence
  NON_STD 2: Xaa = D-Leu
IXRWSRKMPCVS

SEQ ID NO: 45 - protein, 12 aa, Artificial Sequence
  modified peptide derived from HSA Sequence
  NON_STD 2: Xaa = D-Leu
MXRWSRKLPCVS

SEQ ID NO: 46 - protein, 12 aa, Artificial Sequence
  modified peptide derived from HSA Sequence
  NON_STD 2: Xaa = D-Leu
MXRWSRKMPCVS

SEQ ID NO: 47 - protein, 9 aa, Artificial Sequence
  modified peptide derived from HSA Sequence
  MOD_RES 9: Amination
IVRWSKKVP

SEQ ID NO: 48 - protein, 7 aa, Artificial Sequence
  modified peptide derived from HSA Sequence
  MOD_RES 7: Amination
IVRWSKK

SEQ ID NO: 49 - protein, 9 aa, Artificial Sequence
  modified peptide derived from HSA Sequence
  MOD_RES 9: Amination
ILRWSRKLP

SEQ ID NO: 50 - protein, 7 aa, Artificial Sequence
  modified peptide derived from HSA Sequence
  MOD_RES 7: Amination
ILRWSRK

SEQ ID NO: 51 - protein, 12 aa, Artificial Sequence
  modified peptide derived from HSA Sequence
ILRWSRKLPSVS

SEQ ID NO: 52 - protein, 12 aa, Artificial Sequence
  NON_STD 1: Xaa = D-Leu
  modified peptide derived from HSA Sequence
  MOD_RES 7: Palmitic acid with glutamate linker
XLRWSRKMPCVS

SEQ ID NO: 53 - protein, 12 aa, Artificial Sequence
  NON_STD 1: Xaa = D-Leu
  modified peptide derived from HSA Sequence
  MOD_RES 7: Palmitic acid
XLRWSRKMPCVS

SEQ ID NO: 54 - protein, 12 aa, Artificial Sequence
  modified peptide derived from HSA Sequence
  MOD_RES 7: Palmitic acid with Glutamate linker
ILRWSRKLPCVS

SEQ ID NO: 55 - protein, 12 aa, Artificial Sequence
  modified peptide derived from HSA Sequence
  MOD_RES 7: Palmitic acid
ILRWSRKLPCVS

SEQ ID NO: 56 - protein, 12 aa, Artificial Sequence
  modified peptide derived from HSA Sequence
  MOD_RES 11: Palmitic acid with Glutamate linker
ILRWSRKLPCKS

SEQ ID NO: 57 - protein, 12 aa, Artificial Sequence
  modified peptide derived from HSA Sequence
IYRWSRKMPCLS

SEQ ID NO: 58 - protein, 12 aa, Artificial Sequence
  modified peptide derived from HSA Sequence
  MOD_RES 7: Palmitic acid with Glutamate linker
ILRWSRKMPCLS

SEQ ID NO: 59 - protein, 12 aa, Artificial Sequence
  modified peptide derived from HSA Sequence
  MOD_RES 7: Palmitic acid
ILRWSRKMPCLS

SEQ ID NO: 60 - protein, 12 aa, Artificial Sequence
  modified peptide derived from HSA Sequence
IVRWSKKVPSVS

SEQ ID NO: 61 - protein, 7 aa, Artificial Sequence
  modified peptide derived from HSA Sequence
  MOD_RES 6: Palmitic acid
  MOD_RES 7: Amination
IVRWSKK

SEQ ID NO: 62 - protein, 7 aa, Artificial Sequence
  modified peptide derived from HSA Sequence
  MOD_RES 7: Palmitic acid
  MOD_RES 7: Amination
IVRWSKK

SEQ ID NO: 63 - protein, 9 aa, Artificial Sequence
  modified peptide derived from HSA Sequence
  MOD_RES 6: Palmitic acid
  MOD_RES 9: Amination
IVRWSKKVP

SEQ ID NO: 64 - protein, 9 aa, Artificial Sequence
  modified peptide derived from HSA Sequence
  MOD_RES 7: Palmitic acid
  MOD_RES 9: Amination
IVRWSKKVP

SEQ ID NO: 65 - protein, 12 aa, Artificial Sequence
  modified peptide derived from HSA Sequence
  MOD_RES 6: Palmitic acid
IVRWSKKVPCVS

SEQ ID NO: 66 - protein, 12 aa, Artificial Sequence
  modified peptide derived from HSA Sequence
  MOD_RES 7: Palmitic acid
IVRWSKKVPCVS

SEQ ID NO: 67 - protein, 7 aa, Artificial Sequence
  modified peptide derived from HSA Sequence
  MOD_RES 7: Palmitic acid
  MOD_RES 7: Amination
ILRWSRK

SEQ ID NO: 68 - protein, 9 aa, Artificial Sequence
  modified peptide derived from HSA Sequence
  MOD_RES 7: Palmitic acid
  MOD_RES 9: Amination
ILRWSRKLP

SEQ ID NO: 69 - protein, 8 aa, Artificial Sequence
  modified peptide derived from HSA Sequence
  MOD_RES 7: Palmitic acid
  MOD_RES 8: Amination
ILRWSRKL

SEQ ID NO: 70 - protein, 7 aa, Artificial Sequence
  NON_STD 1: Xaa = D-Ile
  modified peptide derived from HSA Sequence
  MOD_RES 7: Amination
XLRWSRK

SEQ ID NO: 71 - protein, 9 aa, Artificial Sequence
  NON_STD 1: Xaa = D-Ile
  modified peptide derived from HSA Sequence
  MOD_RES 9: Amination
XLRWSRKLP

SEQ ID NO: 72 - protein, 9 aa, Artificial Sequence
  NON_STD 1: Xaa = Ile
  modified peptide derived from HSA Sequence
  MOD_RES 7: Palmitic acid
  MOD_RES 9: Amination
XLRWSRKLP

SEQ ID NO: 73 - protein, 12 aa, Artificial Sequence
  NON_STD 1: Xaa = D-Leu
  modified peptide derived from HSA Sequence
  MOD_RES 7: Palmitic acid
XMRWSRKMPCVS

SEQ ID NO: 74 - protein, 12 aa, Artificial Sequence
  modified peptide derived from HSA Sequence
  NON_STD 2: Xaa = D-Leu
  MOD_RES 7: Palmitic acid
MXRWSRKLPCVS

SEQ ID NO: 75 - protein, 9 aa, Artificial Sequence
  NON_STD 1: Xaa = D-Leu
  modified peptide derived from HSA Sequence
  MOD_RES 7: Palmitic acid
  MOD_RES 9: Amination
XMRWSRKMP

SEQ ID NO: 76 - protein, 9 aa, Artificial Sequence
  modified peptide derived from HSA Sequence
  NON_STD 2: Xaa = D-Leu
  MOD_RES 7: Palmitic acid
  MOD_RES 9: Amination
MXRWSRKLP

SEQ ID NO: 77 - protein, 7 aa, Artificial Sequence
  NON_STD 1: Xaa = D-Leu
  modified peptide derived from HSA Sequence
  MOD_RES 7: Palmitic acid
  MOD_RES 7: Amination
XMRWSRK

SEQ ID NO: 78 - protein, 7 aa, Artificial Sequence
  modified peptide derived from HSA Sequence
  NON_STD 2: Xaa = D-Leu
  MOD_RES 7: Palmitic acid
  MOD_RES 7: Amination
MXRWSRK

SEQ ID NO: 79 - protein, 12 aa, Artificial Sequence
  modified peptide derived from HSA Sequence
  MOD_RES 7: Stearic acid
ILRWSRKLPCVS

SEQ ID NO: 80 - protein, 12 aa, Artificial Sequence
  modified peptide derived from HSA Sequence
  MOD_RES 7: Oleic acid
ILRWSRKLPCVS

SEQ ID NO: 81 - protein, 12 aa, Artificial Sequence
  modified peptide derived from HSA Sequence
  MOD_RES 7: Cholesterol
ILRWSRKLPCVS

SEQ ID NO: 82 - protein, 12 aa, Artificial Sequence
  MOD_RES 1: ACETYLATION
  modified peptide derived from HSA Sequence
ILRWSRKLPCVS

SEQ ID NO: 83 - protein, 12 aa, Artificial Sequence
  MOD_RES 1: ACETYLATION
  NON_STD 1: Xaa = D-Ile
  modified peptide derived from HSA Sequence
XLRWSRKLPCVS

SEQ ID NO: 84 - protein, 12 aa, Artificial Sequence
  MOD_RES 1: ACETYLATION
  modified peptide derived from HSA Sequence
MLRWSRKLPCVS

SEQ ID NO: 85 - protein, 12 aa, Artificial Sequence
  MOD_RES 1: ACETYLATION
  NON_STD 1: Xaa = D-Met
  modified peptide derived from HSA Sequence
XLRWSRKLPCVS

SEQ ID NO: 86 - protein, 12 aa, Artificial Sequence
  modified peptide derived from HSA Sequence
VLRWSRKLPCVS

SEQ ID NO: 87 - protein, 12 aa, Artificial Sequence
  NON_STD 1: D-Val
  modified peptide derived from HSA Sequence
XLRWSRKLPCVS

SEQ ID NO: 88 - protein, 8 aa, Artificial Sequence
  modified peptide derived from HSA Sequence
  MOD_RES 8: Palmitic acid with glutamate linker
  MOD_RES 8: Amination
ILRWSRKK

SEQ ID NO: 89 - protein, 7 aa, Artificial Sequence
  modified peptide derived from HSA Sequence
  MOD_RES 7: Palmitic acid with glutamate linker
  MOD_RES 7: Amination
ILRWSRK

SEQ ID NO: 90 - protein, 9 aa, Artificial Sequence
  modified peptide derived from HSA Sequence
  MOD_RES 9: Palmitic acid with glutamate linker
  MOD_RES 9: Amination
ILRWSRKLK

SEQ ID NO: 91 - protein, 7 aa, Artificial Sequence
  NON_STD 1: Xaa = D-Leu
  modified peptide derived from HSA Sequence
  MOD_RES 7: Palmitic acid with glutamate linker
  MOD_RES 7: Amination
XMRWSRK

SEQ ID NO: 92 - protein, 8 aa, Artificial Sequence
  NON_STD 1: D-Leu
  modified peptide derived from HSA Sequence
  MOD_RES 8: Palmitic acid with glutamate linker
  MOD_RES 8: Amination
XMRWSRKK

SEQ ID NO: 93 - protein, 7 aa, Artificial Sequence
  NON_STD 1: Xaa = D-Met
  modified peptide derived from HSA Sequence
  MOD_RES 7: Palmitic acid with glutamate linker
  MOD_RES 7: Amination
XLRWSRK

SEQ ID NO: 94 - protein, 8 aa, Artificial Sequence
  NON_STD 1: Xaa = D-Met
  modified peptide derived from HSA Sequence
  MOD_RES 8: Palmitic acid with glutamate linker
  MOD_RES 8: Amination
XLRWSRKK

SEQ ID NO: 95 - protein, 12 aa, Artificial Sequence
  modified peptide derived from HSA Sequence
  MOD_RES 7: Stearic acid with glutamate linker
ILRWSRKLPCVS

SEQ ID NO: 96 - protein, 13 aa, Artificial Sequence
  modified peptide derived from HSA Sequence
  MOD_RES 8: Stearic acid with glutamate linker
ILRWSRKKLPCVS

SEQ ID NO: 97 - protein, 12 aa, Artificial Sequence
  modified peptide derived from HSA Sequence
  MOD_RES 7: ACETYLATION
ILRWSRKLPCVS

SEQ ID NO: 98 - protein, 12 aa, Artificial Sequence
  modified peptide derived from HSA Sequence
  MOD_RES 7: Decanoic acid with glutamate linker
ILRWSRKLPCVS

SEQ ID NO: 99 - protein, 12 aa, Artificial Sequence
  modified peptide derived from HSA Sequence
  MOD_RES 7: Myristic acid with glutamate linker
ILRWSRKLPCVS

SEQ ID NO: 100 - protein, 12 aa, Artificial Sequence
  modified peptide derived from HSA Sequence
  MOD_RES 7: Stearic acid with glutamate linker
ILRWSRKLPCVS

SEQ ID NO: 101 - protein, 13 aa, Artificial Sequence
  modified peptide derived from HSA Sequence
  MOD_RES 13: dodecane tetraacetic acid
ILRWSRKLPCVSK

SEQ ID NO: 102 - protein, 13 aa, Artificial Sequence
  modified peptide derived from HSA Sequence
  MOD_RES 13: dodecane tetraacetic acid
ILRWSRKLPSVSK

SEQ ID NO: 103 - protein, 26 aa, Artificial Sequence
  Pleurocidin antimicrobial peptide NRC-03
  MOD_RES 26: Amination
GRRKRKWLRRIGKGVKIIGGAALDHL

SEQ ID NO: 104 - protein, 12 aa, Artificial Sequence
  modified peptide derived from HSA Sequence
  MOD_RES 7: Lauric acid with glutamate linker
ILRWSRKLPCVS

SEQ ID NO: 105 - protein, 12 aa, Artificial Sequence
  modified peptide derived from HSA Sequence
  MOD_RES 7: Lauric acid
ILRWSRKLPCVS

SEQ ID NO: 106 - protein, 12 aa, Artificial Sequence
  modified peptide derived from HSA Sequence
  MOD_RES 7: Decanoic acid
ILRWSRKLPCVS

SEQ ID NO: 107 - protein, 12 aa, Artificial Sequence
  modified peptide derived from HSA Sequence
  MOD_RES 7: Myristic acid
ILRWSRKLPCVS

SEQ ID NO: 108 - protein, 12 aa, Artificial Sequence
  modified peptide derived from HSA Sequence
  MOD_RES 7: Oleic acid with glutamate linker
ILRWSRKLPCVS

SEQ ID NO: 109 - protein, 9 aa, Artificial Sequence
  modified peptide derived from HSA Sequence
  DISULFID 5..9: disulfide bond between amino acid 5 and amino acid 9 to form cyclic peptide
  MOD_RES 9: Amination
ILRWCRKPC

SEQ ID NO: 110 - protein, 9 aa, Artificial Sequence
  modified peptide derived from HSA Sequence
  MOD_RES 9: Amination
ILRWCRKPC

SEQ ID NO: 111 - protein, 9 aa, Artificial Sequence
  modified peptide derived from HSA Sequence
  NON_STD 5: Xaa=D-Cys
  DISULFID 5..9: disulfide bond between amino acid 5 and amino acid 9 to form cyclic peptide
  MOD_RES 9: Amination
ILRWXRKPC

SEQ ID NO: 112 - protein, 9 aa, Artificial Sequence
  modified peptide derived from HSA Sequence
  NON_STD 5: Xaa=D-Cys
  MOD_RES 9: Amination
ILRWXRKPC

SEQ ID NO: 113 - protein, 8 aa, Artificial Sequence
  modified peptide derived from HSA Sequence
  NON_STD 5: Xaa=D-Cys
  DISULFID 5..8: disulfide bond between amino acid 5 and amino acid 8 to form cyclic peptide
  MOD_RES 8: Amination
IPRWXRKC

SEQ ID NO: 114 - protein, 8 aa, Artificial Sequence
  modified peptide derived from HSA Sequence
  NON_STD 5: Xaa=D-Cys
  MOD_RES 8: Amination
IPRWXRKC

SEQ ID NO: 115 - protein, 12 aa, Artificial Sequence
  modified peptide derived from HSA Sequence
  SITE 7..12: Peptide bond between amino acid 7 and the carboxyl-terminus to form cyclic peptide
ILRWSRKLPCVS

SEQ ID NO: 116 - protein, 12 aa, Artificial Sequence
  modified peptide derived from HSA Sequence
  SITE 6..12: peptide bond between amino acid 6 and the carboxyl-terminus to form cyclic peptide
ILRWSKKLPCVS

SEQ ID NO: 117 - protein, 12 aa, Artificial Sequence
  modified peptide derived from HSA Sequence
  MOD_RES 7: saturated C16 fatty diacid
ILRWSRKLPCVS

SEQ ID NO: 118 - protein, 12 aa, Artificial Sequence
  modified peptide derived from HSA Sequence
  MOD_RES 7: saturated C16 fatty acid with glutamate linker
ILRWSRKLPCVS

SEQ ID NO: 119 - protein, 12 aa, Artificial Sequence
  modified peptide derived from HSA Sequence
  MOD_RES 7: saturated C18 fatty diacid
ILRWSRKLPCVS

SEQ ID NO: 120 - protein, 12 aa, Artificial Sequence
  modified peptide derived from HSA Sequence
  MOD_RES 7: saturated C18 fatty diacid with glutamate linker
ILRWSRKLPCVS

SEQ ID NO: 121 - protein, 12 aa, Artificial Sequence
  modified peptide derived from HSA Sequence
  MOD_RES 7: Saturated C18 fatty diacid with OEG-OEG-gamma glutamate linker. OEG represents the residue of 8-amino-3,6- dioxaoctanoic acid
ILRWSRKLPCVS

SEQ ID NO: 122 - protein, 8 aa, Artificial Sequence
  modified peptide derived from HSA Sequence
  NON_STD 5: Xaa=D-Cys
  THIOLEST 5..8: thiolester bond between amino acid 5 and amino acid 8 to form cyclic peptide
IPRWXRKP

SEQ ID NO: 123 - protein, 8 aa, Artificial Sequence
  modified peptide derived from HSA Sequence
  NON_STD 5: Xaa=D-Cys
IPRWXRKP

SEQ ID NO: 124 - protein, 8 aa, Artificial Sequence
  modified peptide derived from HSA Sequence
  NON_STD 5: Xaa=D-Cys
  THIOLEST 5..8: thiolester bond between amino acid 5 and amino acid 8 to form cyclic peptide
ILRWXRKP

SEQ ID NO: 125 - protein, 8 aa, Artificial Sequence
  modified peptide derived from HSA Sequence
  NON_STD 5: Xaa=D-Cys
ILRWXRKP

SEQ ID NO: 126 - protein, 8 aa, Artificial Sequence
  modified peptide derived from HSA Sequence
  NON_STD 5: Xaa=D-Ser
  SITE 5..8: ester bond between amino acid 5 and amino acid 8 to form cyclic peptide
IPRWXRKP

SEQ ID NO: 127 - protein, 8 aa, Artificial Sequence
  modified peptide derived from HSA Sequence
  NON_STD 5: Xaa=D-Ser
IPRWXRKP

SEQ ID NO: 128 - protein, 8 aa, Artificial Sequence
  modified peptide derived from HSA Sequence
  NON_STD 5: Xaa=D-Ser
  SITE 5..8: ester bond between amino acid 5 and amino acid 8 to form cyclic peptide
ILRWXRKP

SEQ ID NO: 129 - protein, 8 aa, Artificial Sequence
  modified peptide derived from HSA Sequence
  NON_STD 5: Xaa=D-Ser
ILRWXRKP

SEQ ID NO: 130 - protein, 7 aa, Artificial Sequence
  NON_STD 1: Xaa=D-Leu
  modified peptide derived from HSA Sequence
  MOD_RES 7: Palmitic acid with glutamate linker
  MOD_RES 7: Amination
XLRWSRK

SEQ ID NO: 131 - protein, 7 aa, Artificial Sequence
  NON_STD 1: Xaa=D-Leu
  modified peptide derived from HSA Sequence
  MOD_RES 7: Palmitic acid
  MOD_RES 7: Amination
XLRWSRK

SEQ ID NO: 132 - protein, 7 aa, Artificial Sequence
  NON_STD 1: Xaa=D-Ile
  modified peptide derived from HSA Sequence
  MOD_RES 7: Palmitic acid
  MOD_RES 7: Amination
XLRWSRK

SEQ ID NO: 133 - protein, 7 aa, Artificial Sequence
  NON_STD 1: Xaa=D-Ile
  modified peptide derived from HSA Sequence
  MOD_RES 7: Palmitic acid with glutamate linker
  MOD_RES 7: Amination
XLRWSRK

SEQ ID NO: 134 - protein, 14 aa, Artificial Sequence
  NON_STD 1: Xaa=D-Ile
  modified peptide derived from HSA Sequence
  MOD_RES 11: Palmitic acid
XLRWSRKEYEKEYE

SEQ ID NO: 135 - protein, 9 aa, Artificial Sequence
  NON_STD 1: Xaa=D-Ile
  modified peptide derived from HSA Sequence
  MOD_RES 7: Palmitic acid
  MOD_RES 9: Palmitic acid
XLRWSRKEK

SEQ ID NO: 136 - protein, 9 aa, Artificial Sequence
  modified peptide derived from HSA Sequence
  NON_STD 5: Xaa=D-Cys
  DISULFID 5..9: disulfide bond between amino acid 5 and amino acid 9 to form cyclic peptide
  MOD_RES 7: Palmitic acid
  MOD_RES 9: Amination
ILRWXRKPC

SEQ ID NO: 137 - protein, 9 aa, Artificial Sequence
  NON_STD 1: Xaa=D-Ile
  modified peptide derived from HSA Sequence
  NON_STD 5: Xaa=D-Cys
  DISULFID 5..9: disulfide bond between amino acid 5 and amino acid 9 to form cyclic peptide
  MOD_RES 9: Amination
XLRWXRKPC

SEQ ID NO: 138 - protein, 9 aa, Artificial Sequence
  NON_STD 1: Xaa=D-Ile
  modified peptide derived from HSA Sequence
  NON_STD 5: Xaa=D-Cys
  DISULFID 5..9: disulfide bond between amino acid 5 and amino acid 9 to form cyclic peptide
  MOD_RES 7: Palmitic acid
  MOD_RES 9: Amination
XLRWXRKPC

SEQ ID NO: 139 - protein, 7 aa, Artificial Sequence
  modified peptide derived from HSA Sequence
  MOD_RES 7: saturated C16 fatty diacid
  MOD_RES 7: Amination
ILRWSRK

SEQ ID NO: 140 - protein, 7 aa, Artificial Sequence
  modified peptide derived from HSA Sequence
  MOD_RES 7: saturated C18 fatty diacid
  MOD_RES 7: Amination
ILRWSRK

SEQ ID NO: 141 - protein, 7 aa, Artificial Sequence
  modified peptide derived from HSA Sequence
  MOD_RES 7: saturated C16 fatty diacid with glutamate linker
  MOD_RES 7: Amination
ILRWSRK

SEQ ID NO: 142 - protein, 7 aa, Artificial Sequence
  modified peptide derived from HSA Sequence
  MOD_RES 7: saturated C18 fatty diacid with glutamate linker
  MOD_RES 7: Amination
ILRWSRK

SEQ ID NO: 143 - protein, 7 aa, Artificial Sequence
  NON_STD 1: Xaa=D-Leu
  modified peptide derived from HSA Sequence
  MOD_RES 7: saturated C16 fatty diacid
  MOD_RES 7: Amination
XLRWSRK

SEQ ID NO: 144 - protein, 7 aa, Artificial Sequence
  NON_STD 1: Xaa=D-Leu
  modified peptide derived from HSA Sequence
  MOD_RES 7: saturated C18 fatty diacid
  MOD_RES 7: Amination
XLRWSRK

SEQ ID NO: 145 - protein, 7 aa, Artificial Sequence
  NON_STD 1: Xaa=D-Leu
  modified peptide derived from HSA Sequence
  MOD_RES 7: saturated C16 fatty diacid with glutamate linker
  MOD_RES 7: Amination
XLRWSRK

SEQ ID NO: 146 - protein, 7 aa, Artificial Sequence
  NON_STD 1: Xaa=D-Leu
  modified peptide derived from HSA Sequence
  MOD_RES 7: saturated C18 fatty diacid with glutamate linker
  MOD_RES 7: Amination
XLRWSRK

SEQ ID NO: 147 - protein, 12 aa, Artificial Sequence
  modified peptide derived from HSA Sequence
  MOD_RES 7: saturated C20 fatty acid
ILRWSRKLPCVS

SEQ ID NO: 148 - protein, 12 aa, Artificial Sequence
  modified peptide derived from HSA Sequence
  MOD_RES 7: saturated C20 fatty acid with glutamate linker
ILRWSRKLPCVS

SEQ ID NO: 149 - protein, 7 aa, Artificial Sequence
  modified peptide derived from HSA Sequence
  MOD_RES 7: Stearic acid
  MOD_RES 7: Amination
ILRWSRK

SEQ ID NO: 150 - protein, 7 aa, Artificial Sequence
  modified peptide derived from HSA Sequence
  MOD_RES 7: Stearic acid with glutamate linker
  MOD_RES 7: Amination
ILRWSRK

SEQ ID NO: 151 - protein, 7 aa, Artificial Sequence
  NON_STD 1: Xaa=D-Leu
  modified peptide derived from HSA Sequence
  MOD_RES 7: Stearic acid
  MOD_RES 7: Amination
XLRWSRK

SEQ ID NO: 152 - protein, 7 aa, Artificial Sequence
  NON_STD 1: Xaa=D-Leu
  modified peptide derived from HSA Sequence
  MOD_RES 7: Stearic acid with glutamate linker
  MOD_RES 7: Amination
XLRWSRK

SEQ ID NO: 153 - protein, 9 aa, Artificial Sequence
  modified peptide derived from HSA Sequence
  DISULFID 5..9: Disulfide bond between amino acid 5 and amino acid 9 to form cyclic peptide
  MOD_RES 9: Amination
IMRWCRKPC

SEQ ID NO: 154 - protein, 9 aa, Artificial Sequence
  modified peptide derived from HSA Sequence
  MOD_RES 9: Amination
IMRWCRKPC

SEQ ID NO: 155 - protein, 9 aa, Artificial Sequence
  modified peptide derived from HSA Sequence
  NON_STD 5: Xaa=D-Cys
  DISULFID 5..8: Disulfide bond between amino acid 5 and amino acid 8 to form cyclic peptide
  MOD_RES 9: Amination
IPRWXRKCP

SEQ ID NO: 156 - protein, 9 aa, Artificial Sequence
  modified peptide derived from HSA Sequence
  NON_STD 5: Xaa=D-Cys
  MOD_RES 9: Amination
IPRWXRKCP

SEQ ID NO: 157 - protein, 7 aa, Artificial Sequence
  NON_STD 1: Xaa=D-Leu
  modified peptide derived from HSA Sequence
  MOD_RES 7: Myrisitc acid with glutamate linker
  MOD_RES 7: Amination
XLRWSRK

SEQ ID NO: 158 - protein, 7 aa, Artificial Sequence
  NON_STD 1: Xaa=D-Leu
  modified peptide derived from HSA Sequence
  MOD_RES 7: Myristic acid
  MOD_RES 7: Amination
XLRWSRK

SEQ ID NO: 159 - protein, 7 aa, Artificial Sequence
  modified peptide derived from HSA Sequence
  MOD_RES 7: Myristic acid
  MOD_RES 7: Amination
ILRWSRK

SEQ ID NO: 160 - protein, 7 aa, Artificial Sequence
  modified peptide derived from HSA Sequence
  MOD_RES 7: Myristic acid with glutamate linker
  MOD_RES 7: Amination
ILRWSRK

SEQ ID NO: 161 - protein, 7 aa, Artificial Sequence
  modified peptide derived from HSA Sequenc
  MOD_RES 7: Palmitic acid with glutamate linker
  MOD_RES 7: Amination
LVRYTKK

SEQ ID NO: 162 - protein, 7 aa, Artificial Sequence
  NON_STD 1: Xaa=D-Leu
  modified peptide derived from HSA Sequence
  MOD_RES 7: Palmitic acid with glutamate linker
  MOD_RES 7: Amination
XVRYTKK

SEQ ID NO: 163 - protein, 12 aa, Artificial Sequence
  modified peptide derived from HSA Sequence
  MOD_RES 7: Palmitic acid with glutamate linker
ILRWSRKLPSVS

SEQ ID NO: 164 - protein, 12 aa, Artificial Sequence
  modified peptide derived from HSA Sequence
  MOD_RES 7: Stearic acid with glutamate linker
ILRWSRKLPSVS

SEQ ID NO: 165 - protein, 13 aa, Artificial Sequence
  NON_STD 1: Xaa=D-Leu
  modified peptide derived from HSA Sequence
  MOD_RES 13: Dodecane tetraacetic acid
XLRWSRKMPCVSK

SEQ ID NO: 166 - protein, 7 aa, Artificial Sequence
  modified peptide derived from HSA Sequence
  MOD_RES 7: Dodecane tetraacetic acid
  MOD_RES 7: Amination
ILRWSRK

SEQ ID NO: 167 - protein, 8 aa, Artificial Sequence
  modified peptide derived from HSA Sequence
  MOD_RES 8: Dodecane tetraacetic acid
  MOD_RES 8: Amination
ILRWSRKK

SEQ ID NO: 168 - protein, 7 aa, Artificial Sequence
  NON_STD 1: Xaa=D-Ile
  modified peptide derived from HSA Sequence
  MOD_RES 7: Dodecane tetraacetic acid
  MOD_RES 7: Amination
XLRWSRK

SEQ ID NO: 169 - protein, 8 aa, Artificial Sequence
  NON_STD 1: Xaa=D-Ile
  modified peptide derived from HSA Sequence
  MOD_RES 8: Dodecane tetraacetic acid
  MOD_RES 8: Amination
XLRWSRKK

SEQ ID NO: 170 - protein, 7 aa, Artificial Sequence
  NON_STD 1: Xaa=D-Leu
  modified peptide derived from HSA Sequence
  MOD_RES 7: Palmitic acid with glutamate linker
  MOD_RES 7: Dodecane tetraacetic acid
  MOD_RES 7: Amination
XLRWSRK

SEQ ID NO: 171 - protein, 8 aa, Artificial Sequence
  NON_STD 1: Xaa=D-Leu
  modified peptide derived from HSA Sequence
  MOD_RES 7: Palmitic acid with glutamate linker
  MOD_RES 8: Dodecane tetraacetic acid
  MOD_RES 8: Amination
XLRWSRKK

(19) United States
(12) Patent Application Publication
(10) Pub. No.: US 2023/0131339 A1
(43) Pub. Date: Apr. 27, 2023

(54) EPI-X4 BASED PEPTIDES AND DERIVATIVES THEREOF

(71) Applicants: Universitaet Ulm, Ulm (DE); Baden-Wuerttemberg Stiftung gGmbH, Stuttgart (DE); German University in Cairo, Cairo (EG); Aarhus University, Aarhus (DK); Universitaet Duisburg-Essen, Duisburg (DE)

(72) Inventors: Jan MUENCH, Neu-Ulm (DE); Frank KIRCHHOFF, Ulm-Einsingen (DE); Mirja HARMS, Ulm (DE); Ashraf H. ABADI, Cairo (EG); Monika Maged Wadi HABIB, Cairo (EG); Alexander ZELIKIN, Aarhus N (DK); Sheiliza CARMALI, Aarhus N (DK); Elsa SANCHEZ GARCIA, Bochum (DE); Pandian SOKKAR, Tamil Nadu (IN)

(21) Appl. No.: 17/801,684

(22) PCT Filed: Feb. 26, 2021
(86) PCT No.: PCT/EP2021/054794

§ 371 Date: Aug. 23, 2022

(30) Foreign Application Priority Data
Feb. 27, 2020 (EP) 20159879.4

(51) Int. Cl. C07K 7/08; A61K 47/54; C07K 7/06; A61K 47/10; A61K 47/24; A61K 47/28; A61K 47/32; A61K 47/42

(52) U.S. Cl. CPC C07K 7/08; A61K 47/547; C07K 7/06; A61K 47/10; A61K 47/24; A61K 47/28; A61K 47/32; A61K 47/42

(57) ABSTRACT

A peptide derivative selected from a list of peptide derivatives with a sequence derived from human serum albumin fragment EPI-X4 is provided, which binds to CXCR4 with about 1000-fold stronger efficiency than EPI-X4. The peptide derivatives comprise length variants as well as modifications by length variants, D-amino acids, coupling of fatty acids, cholesterol or polymers, acetyl substitutions of amino groups, amination of carboxyl groups. Further provided are pharmaceutical compositions of the peptide derivative.

### FIELD OF THE INVENTION

The present invention concerns peptides and derivates thereof binding to C-X-C chemokine receptor type 4, therapeutic uses of the peptides and methods for manufacturing the peptides of the invention.

### BACKGROUND OF THE INVENTION

CXC chemokine receptor type 4 (CXCR4) is expressed in many cells of the hematopoietic system, particularly stem cells and tumor cells. CXCR4 is a G protein-coupled receptor (GPCR) with stromal cell-derived factor-1 (SDF-1 or CXCL12) as sole chemokine ligand. CXCR4 is involved in multiple developmental and physiological processes including stem cell homing to the liver and bone marrow as well as participation in organogenesis and healing processes of the organs and wounds. In terms of pathophysiology, CXCR4 plays a role in various disease processes, including tumor growth, cancer cell metastasis, and inflammation. In addition, CXCR4 is a major co-receptor for HIV-1 entry into target cells.

Its involvement in many processes makes CXCR4 an attractive target in intervening with cancer cell proliferation, differentiation, and metastasis as well as inflammatory diseases. So far only one CXCR4 antagonist has obtained clinical approval (AMD3100, Hendrix et al., 2000) but only to mobilize hematopoietic stem cells in cancer patients with lymphoma and multiple myeloma.

A peptide derived from Human Serum Albumin amino acid sequence (EPI-X4 (SEQ ID NO.: 1)) has been shown to bind CXCR4, thereby inhibiting the binding of natural ligand CXCL12 (EP 2 162 462 B1). Peptides derived from EPI-X4 (SEQ ID NO.: 1) have been shown to bind CXCR4 more effectively than the original peptide (EP 3 007 717 A1). Yet, effective inhibition of binding of CXCL12 requests higher nanomolar values of these peptides, and the half-life period of the peptides is limited due to degradation by protease activity. There is a need for CXCR4 antagonists binding CXCR4 more effectively and with an increased blood stability and pharmacokinetic properties.

### SUMMARY OF THE INVENTION

A first aspect of the present invention is related to a peptide consisting of one of the following amino acid sequences, selected from any of the groups 1 to 11, wherein
Group 7 consists of

(SEQ ID NO.: 70, JM#173)
d-ILRWSRK-NH2
(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 71, JM#174)
d-ILRWSRKLP-NH2
(SEQ ID NO.: 95, JM#198)
ILRWSRK(Glu-Ste)LPCVS
(SEQ ID NO.: 104, JM#213)
ILRWSRK(Glu-Lau)LPCVS
(SEQ ID NO.: 108, JM#217)
ILRWSRK(Glu-Ole)LPCVS
(SEQ ID NO.: 117, JM#226)
ILRWSRK(C16diacid)LPCVS
(SEQ ID NO.: 118, JM#227)
ILRWSRK(Glu-C16diacid)LPCVS
(SEQ ID NO.: 119, JM#228)
ILRWSRK(C18diacid)LPCVS
(SEQ ID NO.: 120, JM#229)
ILRWSRK(Glu-C18diacid)LPCVS
(SEQ ID NO.: 121, JM#230)
ILRWSRK(OEG-OEG-γGlu-C18diacid)LPCVS
(SEQ ID NO.: 130, JM#235)
d-LLRWSRK(Glu-Pal)-NH2
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 133, JM#238)
d-ILRWSRK(Glu-Pal)-NH2
(SEQ ID NO.: 139, JM#244)
ILRWSRK(C16diacid)-NH2
(SEQ ID NO.: 140, JM#245)
ILRWSRK(C18diacid)-NH2
(SEQ ID NO.: 141, JM#246)
ILRWSRK(Glu-C16diacid)-NH2
(SEQ ID NO.: 142, JM#247)
ILRWSRK(Glu-C18diacid)-NH2
(SEQ ID NO.: 143, JM#248)
d-LLRWSRK(C16diacid)-NH2
(SEQ ID NO.: 144, JM#249)
d-LLRWSRK(C18diacid)-NH2
(SEQ ID NO.: 145, JM#250)
d-LLRWSRK(Glu-C16diacid)-NH2
(SEQ ID NO.: 146, JM#251)
d-LLRWSRK(Glu-C18diacid)-NH2
(SEQ ID NO.: 147, JM#252)
ILRWSRK(Ara)LPCVS
(SEQ ID NO.: 148, JM#253)
ILRWSRK(Glu-Ara)LPCVS
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 151, JM#256)
d-LLRWSRK(Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2
(SEQ ID NO.: 159, JM#262)
ILRWSRK(Myr)-NH2
(SEQ ID NO.: 161, JM#264)
LVRYTKK(Glu-Pal)-NH2
(SEQ ID NO.: 162, JM#265)
d-LVRYTKK(Glu-Pal)-NH2
(SEQ ID NO.: 163)
ILRWSRK(Pal-Glu)LPSVS

Group 1 consists of

(SEQ ID NO.: 20, JM#18)
ILRWSRKMPCLS
(SEQ ID NO.: 55, JM#144)
ILRWSRK(Pal)LPCVS
(SEQ ID NO.: 89, JM#192)
ILRWSRK(Glu-Pal)-NH2
(SEQ ID NO.: 91, JM#194)
d-LMRWSRK(Glu-Pal)-NH2
(SEQ ID NO.: 150, JM#255)
ILRWSRK(Glu-Ste)-NH2

Group 2 consists of

(SEQ ID NO.: 69, JM#172)
ILRWSRK(Pal)L-NH2
(SEQ ID NO.: 75, JM#178)
d-LMRWSRK(Pal)MP-NH2
(SEQ ID NO.: 77, JM#180)
d-LMRWSRK(Pal)-NH2
(SEQ ID NO.: 80, JM#183)
ILRWSRK(Ole)LPCVS
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 152, JM#257)
d-LLRWSRK(Glu-Ste)-NH2

Group 3 consists of

(SEQ ID NO.: 53, JM#141)
d-LLRWSRK(Pal)MPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 66, JM#169)
IVRWSKK(Pal)VPCVS
(SEQ ID NO.: 67, JM#170)
ILRWSRK(Pal)-NH2
(SEQ ID NO.: 68, JM#171)
ILRWSRK(Pal)LP-NH2
(SEQ ID NO.: 88, JM#191)
ILRWSRKK(Glu-Pal)-NH2
(SEQ ID NO.: 90, JM#193)
ILRWSRKLK(Glu-Pal)-NH2
(SEQ ID NO.: 96, JM#199)
ILRWSRKK(Glu-Ste)LPCVS
(SEQ ID NO.: 98, JM#203)
ILRWSRK(Glu-Dec)LPCVS
(SEQ ID NO.: 100, JM#205)
ILRWSRK(Glu-Ste)LPCVS
(SEQ ID NO.: 107, JM#216)
ILRWSRK(Myr)LPCVS
(SEQ ID NO.: 149, JM#254)
ILRWSRK(Ste)-NH2
(SEQ ID NO.: 157, JM#260)
d-LLRWSRK(Glu-Myr)-NH2
(SEQ ID NO.: 160, JM#263)
ILRWSRK(Glu-Myr)-NH2

Group 4 consists of

(SEQ ID NO.: 10, JM#8)
ILRWSRKVPCVS
(SEQ ID NO.: 12, JM#10)
IFRWSRKVPCVS
(SEQ ID NO.: 29, JM#27)
MLRWSRKMPCVS
(SEQ ID NO.: 36, JM#34)
MMRWSRKMPCVS
(SEQ ID NO.: 41, JM#39)
MLRWSRKLPCVS
(SEQ ID NO.: 51, JM#122)
ILRWSRKLPSVS
(SEQ ID NO.: 52, JM#140)
d-LLRWSRK(Glu-Pal)MPCVS
(SEQ ID NO.: 59, JM#149)
ILRWSRK(Pal)MPCLS
(SEQ ID NO. 92, JM#195)
d-LMRWSRKK(Glu-Pal)-NH2
(SEQ ID NO.: 97, JM#200)
ILRWSRK-AcLPCVS
(SEQ ID NO.: 105, JM#214)
ILRWSRK(Lau)LPCVS
(SEQ ID NO.: 151, JM#256)
d-LLRWSRK(Ste)-NH2
(SEQ ID NO.: 159, JM#262)
ILRWSRK(Myr)-NH2

Group 5 consists of

(SEQ ID NO. 3, JM#1)
ILRWSKKVPCVS
(SEQ ID NO. 4, JM#2)
IFRWSKKVPCVS
(SEQ ID NO. 5, JM#3)
IVRWSRKVPCVS
(SEQ ID NO. 6, JM#4)
IVRWSHKVPCVS
(SEQ ID NO. 7, JM#5)
IVRWSKKLPCVS
(SEQ ID NO. 8, JM#6)
IVRWSKKIPCVS
(SEQ ID NO. 9, JM#7)
IVRWSKKFPCVS
(SEQ ID NO. 11, JM#9)
ILRWSHKVPCVS
(SEQ ID NO. 13, JM#11)
IFRWSHKVPCVS
(SEQ ID NO. 14, JM#12)
IVRWSKKMPCVS
(SEQ ID NO. 16, JM#14)
IVRWSKKVPCd-VS
(SEQ ID NO. 17, JM#15)
ILRWSRKVPCd-VS
(SEQ ID NO. 18, JM#16)
IIRWSRKMPCVS
(SEQ ID NO. 25, JM#23)
ILRWSRKVPSVS
(SEQ ID NO. 26, JM#24)
ILRWSRKMPSVS
(SEQ ID NO. 27, JM#25)
Ac-SLRWSRKMPCVS
(SEQ ID NO. 28, JM#26)
d-Ac-SLRWSRKMPCVS
(SEQ ID NO. 30, JM#28)
d-MLRWSRKMPCVS
(SEQ ID NO. 31, JM#29)
d-LLRWSRKMPCVS
(SEQ ID NO. 32, JM#30)
d-FLRWSRKMPCVS
(SEQ ID NO. 33, JM#31)
GLRWSRKMPCVS
(SEQ ID NO. 34, JM#32)
Ac-SMRWSRKMPCVS
(SEQ ID NO. 35, JM#33)
d-Ac-SMRWSRKMPCVS
(SEQ ID NO. 37, JM#35)
d-MMRWSRKMPCVS
(SEQ ID NO. 38, JM#36)
d-LMRWSRKMPCVS
(SEQ ID NO. 39, JM#37)
d-FMRWSRKMPCVS
(SEQ ID NO. 40, JM#38)
d-GMRWSRKMPCVS
(SEQ ID NO. 42, JM#40)
d-MLRWSRKLPCVS
(SEQ ID NO. 43, JM#41)
Id-LRWSRKLPCVS
(SEQ ID NO. 44, JM#42)
Id-LRWSRKMPCVS
(SEQ ID NO. 45, JM#43)
Md-LRWSRKLPCVS
(SEQ ID NO. 46, JM#44)
Md-LRWSRKMPCVS
(SEQ ID NO. 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS
(SEQ ID NO.: 106, JM#215)
ILRWSRK(Dec)LPCVS
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 158, JM#261)
d-LLRWSRK(Myr)-NH2
(SEQ ID NO.: 163)
ILRWSRK(Pal-Glu)LPSVS
(SEQ ID NO.: 164)
ILRWSRK(Glu-Ste)LPSVS

Group 6 consists of

(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 164)
ILRWSRK(Glu-Ste)LPSVS

Group 8 consists of

(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2

Group 9 consists of

(SEQ ID NO.: 27, JM#25)
Ac-SLRWSRKMPCVS
(SEQ ID NO.: 30, JM#28)
d-MLRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 52, JM#140)
d-LLRWSRK(Glu-Pal)MPCVS
(SEQ ID NO.: 75, JM#178)
d-LMRWSRK(Pal)MP-NH2
(SEQ ID NO.: 106, JM#215)
ILRWSRK(Dec)LPCVS
(SEQ ID NO.: 158, JM#261)
d-LLRWSRK(Myr)-NH2
(SEQ ID NO.: 160, JM#263)
ILRWSRK(Glu-Myr)-NH2

Group 10 consists of

(SEQ ID NO.: 55, JM#144)
ILRWSRK(Pal)LPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 69, JM#172)
ILRWSRK(Pal)L-NH2
(SEQ ID NO.: 77, JM#180)
d-LMRWSRK(Pal)-NH2
(SEQ ID NO.: 88, JM#191)
ILRWSRKK(Glu-Pal)-NH2
(SEQ ID NO.: 89, JM#192)
ILRWSRK(Glu-Pal)-NH2
(SEQ ID NO.: 90, JM#193)
ILRWSRKLK(Glu-Pal)-NH2
(SEQ ID NO.: 91, JM#194)
d-LMRWSRK(Glu-Pal)-NH2
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2

Group 11 consists of
ILRWCRKPC-NH2, wherein the cysteine at position 5 is bound to the cysteine at position 9 via a disulfide bond to form a cyclic peptide (SEQ ID NO.: 109, JM #218)
ILRW(d-C)RKPC-NH2, wherein the d-cysteine at position 5 is bound to the cysteine at position 9 via a disulfide bond to form a cyclic peptide (SEQ ID NO.: 111, JM #219)
IPRW(d-C)RKC-NH2, wherein the d-cysteine at position 5 is bound to the cysteine at position 8 via a disulfide bond to form a cyclic peptide (SEQ ID NO.: 113, JM #220)
ILRWSRKLPCVS, wherein the lysine at position 7 is bound to the carboxyl-terminus via a peptide bond to form a cyclic peptide (SEQ ID NO.: 115, JM #221)
ILRWSKKLPCVS, wherein the lysine at position 6 is bound to the carboxyl-terminus via a peptide bond to form a cyclic peptide (SEQ ID NO.: 116, JM #222)
IPRW(d-C)RKP, wherein the d-cysteine at position 5 is bound to the proline at position 8 via a thioester bond to form a cyclic peptide (SEQ ID NO.: 122, JM #231)
ILRW(d-C)RKP, wherein the d-cysteine at position 5 is bound to the proline at position 8 via a thioester bond to form a cyclic peptide (SEQ ID NO.: 124, JM #232)
IPRW(d-S)RKP, wherein the d-serine at position 5 is bound to the proline at position 8 via an ester bond to form a cyclic peptide (SEQ ID NO.: 126, JM #233)
ILRW(d-S)RKP, wherein the d-serine at position 5 is bound to the proline at position 8 via an ester bond to form a cyclic peptide (SEQ ID NO.: 128, JM #234)
IMRWCRKPC-NH2, wherein the cysteine at position 5 is bound to the cysteine at position 9 via a disulfide bond to form a cyclic peptide (SEQ ID NO.: 153, JM #258)
IPRW(d-C)RKCP-NH2, wherein the d-cysteine at position 5 is bound to the cysteine at position 8 via a disulfide bond to form a cyclic peptide (SEQ ID NO.: 155, JM #259)
wherein d- in front of an amino acid indicates a D-amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol on the preceding amino acid, Ac indicates a substitution of an amino group by an acetyl group, Lau indicates lauric acid on the preceding amino acid, Glu-Lau indicates a lauric acid on the preceding amino acid with a glutamate linker, Glu-Ole indicates an oleic acid on the preceding amino acid with a glutamate linker, C16diacid indicates a saturated C16 fatty diacid on the preceding amino acid, Glu-C16diacid indicates a saturated C16 fatty diacid on the preceding amino acid with a glutamate linker, C18diacid indicates a saturated C18 fatty diacid on the preceding amino acid, Glu-C18diacid indicates a saturated C18 fatty diacid on the preceding amino acid with a glutamate linker, OEG-OEG-γGlu-C18diacid indicates a saturated C18 fatty diacid on the preceding amino acid with an OEG-OEG-gamma glutamate linker, OEG represents the residue of 8-amino-3,6-dioxaoctanoic acid, Ara indicates a saturated C20 fatty acid on the preceding amino acid, and Glu-Ara indicates a saturated C20 fatty acid on the preceding amino acid with a glutamate linker.

A second aspect of the invention is directed to a conjugate in which the peptide according to the invention is conjugated to a complexing agent.

A third aspect of the invention is directed to a conjugate in which the peptide according to the invention is coupled to a polymer.

A fourth aspect of the invention is related to a peptide consisting of two identical monomeric peptides according to the invention, wherein the monomeric peptides are linked to each other via a cysteine bridge which is formed between the monomeric peptides to form a dimeric peptide.

A fifth aspect of the invention is related to a pharmaceutical composition comprising the inventive peptide together with at least one pharmaceutically acceptable carrier, mesoporous nanoparticles, cryoprotectant, lyoprotectant, excipient and/or diluent.

A sixth aspect of the invention is related to the inventive peptide or the inventive pharmaceutical composition for use in medicine.

A seventh aspect of the invention is related to the use of the inventive peptide or the inventive pharmaceutical composition for the preparation of a formulation for oral administration, inhalation, intravenous administration, topical administration, intranasal administration, intraperitoneal administration, subcutaneous administration and/or any other injectable form.

An eighth aspect of the invention is related to the inventive peptide or the inventive pharmaceutical composition for use in the treatment of disorders of hematopoiesis, in the treatment of wounds, in the treatment of viral diseases, in particular infections with HIV-1, HIV-2, SARS-CoV-2, Cytomegalovirus, Herpes simplex virus (type 1 and 2), Varicella zoster virus, Hepatitis A and Hepatitis B virus, Influenza virus, Polio virus, Rhino virus, Rubella virus, Measles virus, Rabies virus, Rous sarcoma virus, Epstein-Barr Virus; in the treatment of infections caused by bacteria and fungi, in particular Pseudomonas, Candida, S. aureus, in the treatment of infectious processes, abnormal infectious processes, in the treatment of inflammation, in particular of periodontal disease, arthritis and inflammatory bowel disease, as well as dermatitis and asthma; in the treatment of growth disorders, in the treatment of neuronal diseases, disorders of the blood clotting cascade and hematopoiesis, vascular diseases, diseases of the immune system, for improving wound and bone healing, for use in the treatment of neurological diseases, in particular stroke, Parkinson's disease, Alzheimer's disease, multiple sclerosis, in the treatment of warts, Hypogammaglobulinemia, Immunodeficiency, and Myelokathexis syndrome (WHIM-syndrome) and rheumatoid arthritis; in the treatment of cancers, in particular cancers showing the CXCR4 receptor such as cancer of the liver, pancreas, prostate, breast cancer or other solid tumors, in the treatment of lack of mobilization, proliferation and migration of stem cells, T-cell activation as well as support of immunoblasts, such as CTL/PD-1, in the treatment of antifibrosis; in the treatment or prevention of scars; in the treatment of cardiologic disorders, in the treatment of metabolic disorders, in particular diabetes, and in the treatment of lung diseases, in particular lung fibrosis, bronchitis, chronic obstructive pulmonary disease (COPD).

A ninth aspect of the invention is related to the inventive peptide or the inventive pharmaceutical composition for use in the prophylaxis and/or treatment of cancer, viral diseases, metabolic disorders, neurologic disorders, diseases of the immune system, or disorders of the blood clotting cascade and hematopoiesis in a mammal, wherein the mammal preferably is a human.

A tenth aspect of the invention is related to a method for manufacturing the inventive peptide by solid phase synthesis.

A further aspect of the invention is related to a conjugate in which the peptide according to the invention is coupled to cholesterol.

A further aspect of the invention is related to a conjugate in which the peptide according to the invention is coupled to a drug.

A further aspect of the invention is related to a conjugate in which the peptide according to the invention is coupled to human serum albumin.

### BRIEF DESCRIPTION OF THE FIGURES

FIG. 1 shows two diagrams of X4-HIV-1 assays, wherein infection rates of cells are depending on concentrations of different peptide derivatives.

FIG. 2 shows two diagrams of antibody competition assays, wherein the percentage of bound antibody is dependent on concentrations of truncated palmitoylated variants of peptide JM #21 (SEQ ID NO.: 23) (A) and of truncated palmitoylated variants of peptide JM #21 (SEQ ID NO.: 23) which are terminally modified (B).

FIG. 3 shows a diagram of inhibition of CXCL12-induced Akt and Erk signaling, wherein the percentage of phosphorylated Akt and Erk is depending on concentrations of different peptide derivatives.

FIG. 4 shows diagrams of stability of different peptides in whole human plasma.

FIG. 5 shows a diagram of antibody competition assays, wherein the percentage of bound antibody is depending on concentrations of DOTA-coupled peptides.

FIG. 6 shows two diagrams of X4-HIV-1 assays, wherein infection rates of cells are depending on concentrations of different peptides which are coupled to polyethylene glycol (A) or poly(vinyl alcohol) and poly(vinyl pyrrolidone) (B).

FIG. 7 shows two diagrams of antibody competition assays, wherein the percentage of bound antibody is dependent on concentrations of truncated palmitoylated variants of peptides which are coupled to polyethylene glycol (A) or poly(vinyl alcohol) and poly(vinyl pyrrolidone) (B).

FIG. 8 shows two diagrams of assays testing the activity of DSPE-coupled peptides, wherein (A) shows a diagram of X4-HIV-1 assays, and (B) shows a diagram of antibody competition assays.

FIG. 9 shows diagrams illustrating the inhibition of CXCL12 induced Ca²⁺-signalling by A) JM #21 (SEQ ID NO.: 23) and peptide variants thereof and B) WSC02 (SEQ ID NO.: 2) and peptide variants thereof.

FIG. 10 shows diagrams illustrating the inhibition of CXCL12 induced T cell migration by A) peptide JM #21 (SEQ ID NO.: 23) compared to peptides of the state of the art EPIX4 (SEQ ID NO.: 1) and WSC02 (SEQ ID NO.: 2) and B) short peptides variants of JM #21 (SEQ ID NO.: 23) and WSC02 (SEQ ID NO.: 2) and C) fatty acid coupled peptide variants of JM #21 (SEQ ID NO.: 23) and WSC02 (SEQ ID NO.: 2).

FIG. 11 shows illustrations of the computational models developed for the design of novel peptide derivatives: A) peptide-protein model in explicit water and membrane environment, B) investigation of tentative binding sites, C) analysis of the energy contributions and D) molecular interactions in the binding site.

FIG. 12 shows diagrams of stability of different peptides in human S9 liver fractions.

FIG. 13 shows a diagram of in vivo stability of different peptides.

FIG. 14 shows a diagram of cellular uptake and distribution of ¹⁷⁷Lu-labeled DOTA-conjugated peptides and ¹⁷⁷Lu-labeled Pentixather in GHOST-CXCR4+ cells.

FIG. 15 shows a diagram of cellular uptake and distribution of a ¹⁷⁷Lu/⁶⁸Ga-labeled DOTA-conjugated peptide and ¹⁷⁷Lu/⁶⁸Ga-labeled Pentixather in GHOST-CXCR4+ cells.

FIG. 16 shows a diagram of cellular uptake of a ¹⁷⁷Lu-labeled DOTA-conjugated peptide and ¹⁷⁷Lu-labeled Pentixather in Jurkat cells.

### DETAILED DESCRIPTION OF THE INVENTION

A first aspect of the present invention is directed to a peptide consisting of one of the following amino acid sequences, selected from any of the groups 1 to 11, wherein
Group 7 consists of

(SEQ ID NO.: 70, JM#173)
d-ILRWSRK-NH2
(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 71, JM#174)
d-ILRWSRKLP-NH2
(SEQ ID NO.: 95, JM#198)
ILRWSRK(Glu-Ste)LPCVS
(SEQ ID NO.: 104, JM#213)
ILRWSRK(Glu-Lau)LPCVS
(SEQ ID NO.: 108, JM#217)
ILRWSRK(Glu-Ole)LPCVS
(SEQ ID NO.: 117, JM#226)
ILRWSRK(C16diacid)LPCVS
(SEQ ID NO.: 118, JM#227)
ILRWSRK(Glu-C16diacid)LPCVS
(SEQ ID NO.: 119, JM#228)
ILRWSRK(C18diacid)LPCVS
(SEQ ID NO.: 120, JM#229)
ILRWSRK(Glu-C18diacid)LPCVS
(SEQ ID NO.: 121, JM#230)
ILRWSRK(OEG-OEG-YGlu-C18diacid)LPCVS
(SEQ ID NO.: 130, JM#235)
d-LLRWSRK(Glu-Pal)-NH2
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 133, JM#238)
d-ILRWSRK(Glu-Pal)-NH2
(SEQ ID NO.: 139, JM#244)
ILRWSRK(C16diacid)-NH2
(SEQ ID NO.: 140, JM#245)
ILRWSRK(C18diacid)-NH2
(SEQ ID NO.: 141, JM#246)
ILRWSRK(Glu-C16diacid)-NH2
(SEQ ID NO.: 142, JM#247)
ILRWSRK(Glu-C18diacid)-NH2
(SEQ ID NO.: 143, JM#248)
d-LLRWSRK(C16diacid)-NH2
(SEQ ID NO.: 144, JM#249)
d-LLRWSRK(C18diacid)-NH2
(SEQ ID NO.: 145, JM#250)
d-LLRWSRK(Glu-C16diacid)-NH2
(SEQ ID NO.: 146, JM#251)
d-LLRWSRK(Glu-C18diacid)-NH2
(SEQ ID NO.: 147, JM#252)
ILRWSRK(Ara)LPCVS
(SEQ ID NO.: 148, JM#253)
ILRWSRK(Glu-Ara)LPCVS
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 151, JM#256)
d-LLRWSRK(Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2
(SEQ ID NO.: 159, JM#262)
ILRWSRK(Myr)-NH2
(SEQ ID NO.: 161, JM#264)
LVRYTKK(Glu-Pal)-NH2
(SEQ ID NO.: 162, JM#265)
d-LVRYTKK(Glu-Pal)-NH2
(SEQ ID NO.: 163)
ILRWSRK(Pal-Glu)LPSVS

Group 1 consists of

(SEQ ID NO.: 20, JM#18)
ILRWSRKMPCLS
(SEQ ID NO.: 55, JM#144)
ILRWSRK(Pal)LPCVS
(SEQ ID NO.: 89, JM#192)
ILRWSRK(Glu-Pal)-NH2
(SEQ ID NO.91, JM#194)
d-LMRWSRK(Glu-Pal)-NH2
(SEQ ID NO.: 150, JM#255)
ILRWSRK(Glu-Ste)-NH2

Group 2 consists of

(SEQ ID NO.: 69, JM#172)
ILRWSRK(Pal)L-NH2
(SEQ ID NO.: 75, JM#178)
d-LMRWSRK(Pal)MP-NH2
(SEQ ID NO.: 77, JM#180)
d-LMRWSRK(Pal)-NH2
(SEQ ID NO.: 80, JM#183)
ILRWSRK(Ole)LPCVS
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 152, JM#257)
d-LLRWSRK(Glu-Ste)-NH2

Group 3 consists of

(SEQ ID NO.: 53, JM#141)
d-LLRWSRK(Pal)MPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 66, JM#169)
IVRWSKK(Pal)VPCVS
(SEQ ID NO.: 67, JM#170)
ILRWSRK(Pal)-NH2
(SEQ ID NO.: 68, JM#171)
ILRWSRK(Pal)LP-NH2
(SEQ ID NO.: 88, JM#191)
ILRWSRKK(Glu-Pal)-NH2
(SEQ ID NO.: 90, JM#193)
ILRWSRKLK(Glu-Pal)-NH2
(SEQ ID NO.: 96, JM#199)
ILRWSRKK(Glu-Ste)LPCVS
(SEQ ID NO.: 98, JM#203)
ILRWSRK(Glu-Dec)LPCVS
(SEQ ID NO.: 100, JM#205)
ILRWSRK(Glu-Ste)LPCVS
(SEQ ID NO.: 107, JM#216)
ILRWSRK(Myr)LPCVS
(SEQ ID NO.: 149, JM#254)
ILRWSRK(Ste)-NH2
(SEQ ID NO.: 157, JM#260)
d-LLRWSRK(Glu-Myr)-NH2
(SEQ ID NO.: 160, JM#263)
ILRWSRK(Glu-Myr)-NH2

Group 4 consists of

(SEQ ID NO.: 10, JM#8)
ILRWSRKVPCVS
(SEQ ID NO.: 12, JM#10)
IFRWSRKVPCVS
(SEQ ID NO.: 29, JM#27)
MLRWSRKMPCVS
(SEQ ID NO.: 36, JM#34)
MMRWSRKMPCVS
(SEQ ID NO.: 41, JM#39)
MLRWSRKLPCVS
(SEQ ID NO.: 51, JM#122)
ILRWSRKLPSVS
(SEQ ID NO.: 52, JM#140)
d-LLRWSRK(Glu-Pal)MPCVS
(SEQ ID NO.: 59, JM#149)
ILRWSRK(Pal)MPCLS
(SEQ ID NO.: 92, JM#195)
d-LMRWSRKK(Glu-Pal)-NH2
(SEQ ID NO.: 97, JM#200)
ILRWSRK-AcLPCVS
(SEQ ID NO.: 105, JM#214)
ILRWSRK(Lau)LPCVS
(SEQ ID NO.: 151, JM#256)
d-LLRWSRK(Ste)-NH2
(SEQ ID NO.: 159, JM#262)
ILRWSRK(Myr)-NH2

Group 5 consists of

(SEQ ID NO. 3, JM#1)
ILRWSKKVPCVS
(SEQ ID NO. 4, JM#2)
IFRWSKKVPCVS
(SEQ ID NO. 5, JM#3)
IVRWSRKVPCVS
(SEQ ID NO. 6, JM#4)
IVRWSHKVPCVS
(SEQ ID NO. 7, JM#5)
IVRWSKKLPCVS
(SEQ ID NO. 8, JM#6)
IVRWSKKIPCVS
(SEQ ID NO. 9, JM#7)
IVRWSKKFPCVS
(SEQ ID NO. 11, JM#9)
ILRWSHKVPCVS
(SEQ ID NO. 13, JM#11)
IFRWSHKVPCVS
(SEQ ID NO. 14, JM#12)
IVRWSKKMPCVS
(SEQ ID NO. 16, JM#14)
IVRWSKKVPCd-VS
(SEQ ID NO. 17, JM#15)
ILRWSRKVPCd-VS
(SEQ ID NO. 18, JM#16)
IIRWSRKMPCVS
(SEQ ID NO. 25, JM#23)
ILRWSRKVPSVS
(SEQ ID NO. 26, JM#24)
ILRWSRKMPSVS
(SEQ ID NO. 27, JM#25)
Ac-SLRWSRKMPCVS
(SEQ ID NO. 28, JM#26)
d-Ac-SLRWSRKMPCVS
(SEQ ID NO. 30, JM#28)
d-MLRWSRKMPCVS
(SEQ ID NO. 31, JM#29)
d-LLRWSRKMPCVS
(SEQ ID NO. 32, JM#30)
d-FLRWSRKMPCVS
(SEQ ID NO. 33, JM#31)
GLRWSRKMPCVS
(SEQ ID NO. 34, JM#32)
Ac-SMRWSRKMPCVS
(SEQ ID NO. 35, JM#33)
d-Ac-SMRWSRKMPCVS
(SEQ ID NO. 37, JM#35)
d-MMRWSRKMPCVS
(SEQ ID NO. 38, JM#36)
d-LMRWSRKMPCVS
(SEQ ID NO. 39, JM#37)
d-FMRWSRKMPCVS
(SEQ ID NO. 40, JM#38)
d-GMRWSRKMPCVS
(SEQ ID NO. 42, JM#40)
d-MLRWSRKLPCVS
(SEQ ID NO. 43, JM#41)
Id-LRWSRKLPCVS
(SEQ ID NO. 44, JM#42)
Id-LRWSRKMPCVS
(SEQ ID NO. 45, JM#43)
Md-LRWSRKLPCVS
(SEQ ID NO. 46, JM#44)
Md-LRWSRKMPCVS
(SEQ ID NO. 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS
(SEQ ID NO.: 106, JM#215)
ILRWSRK(Dec)LPCVS
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 158, JM#261)
d-LLRWSRK(Myr)-NH2
(SEQ ID NO.: 163)
ILRWSRK(Pal-Glu)LPSVS
(SEQ ID NO.: 164)
ILRWSRK(Glu-Ste)LPSVS

Group 6 consists of

(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 164)
ILRWSRK(Glu-Ste)LPSVS

Group 8 consists of

(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2

Group 9 consists of

(SEQ ID NO.: 27, JM#25)
Ac-SLRWSRKMPCVS
(SEQ ID NO.: 30, JM#28)
d-MLRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 52, JM#140)
d-LLRWSRK(Glu-Pal)MPCVS
(SEQ ID NO.: 75, JM#178)
d-LMRWSRK(Pal)MP-NH2
(SEQ ID NO.: 106, JM#215)
ILRWSRK(Dec)LPCVS
(SEQ ID NO.: 158, JM#261)
d-LLRWSRK(Myr)-NH2
(SEQ ID NO.: 160, JM#263)
ILRWSRK(Glu-Myr)-NH2

Group 10 consists of

(SEQ ID NO.: 55, JM#144)
ILRWSRK(Pal)LPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 69, JM#172)
ILRWSRK(Pal)L-NH2
(SEQ ID NO.: 77, JM#180)
d-LMRWSRK(Pal)-NH2
(SEQ ID NO.: 88, JM#191)
ILRWSRKK(Glu-Pal)-NH2
(SEQ ID NO.: 89, JM#192)
ILRWSRK(Glu-Pal)-NH2
(SEQ ID NO.: 90, JM#193)
ILRWSRKLK(Glu-Pal)-NH2
(SEQ ID NO.: 91, JM#194)
d-LMRWSRK(Glu-Pal)-NH2
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2

Group 11 consists of
ILRWCRKPC-NH2, wherein the cysteine at position 5 is bound to the cysteine at position 9 via a disulfide bond to form a cyclic peptide (SEQ ID NO.: 109, JM #218)
ILRW(d-C)RKPC-NH2, wherein the d-cysteine at position 5 is bound to the cysteine at position 9 via a disulfide bond to form a cyclic peptide (SEQ ID NO.: 111, JM #219)
IPRW(d-C)RKC-NH2, wherein the d-cysteine at position 5 is bound to the cysteine at position 8 via a disulfide bond to form a cyclic peptide (SEQ ID NO.: 113, JM #220)
ILRWSRKLPCVS, wherein the lysine at position 7 is bound to the carboxyl-terminus via a peptide bond to form a cyclic peptide (SEQ ID NO.: 115, JM #221)
ILRWSKKLPCVS, wherein the lysine at position 6 is bound to the carboxyl-terminus via a peptide bond to form a cyclic peptide (SEQ ID NO.: 116, JM #222)
IPRW(d-C)RKP, wherein the d-cysteine at position 5 is bound to the proline at position 8 via a thioester bond to form a cyclic peptide (SEQ ID NO.: 122, JM #231)
ILRW(d-C)RKP, wherein the d-cysteine at position 5 is bound to the proline at position 8 via a thioester bond to form a cyclic peptide (SEQ ID NO.: 124, JM #232)
IPRW(d-S)RKP, wherein the d-serine at position 5 is bound to the proline at position 8 via an ester bond to form a cyclic peptide (SEQ ID NO.: 126, JM #233)
ILRW(d-S)RKP, wherein the d-serine at position 5 is bound to the proline at position 8 via an ester bond to form a cyclic peptide (SEQ ID NO.: 128, JM #234)
IMRWCRKPC-NH2, wherein the cysteine at position 5 is bound to the cysteine at position 9 via a disulfide bond to form a cyclic peptide (SEQ ID NO.: 153, JM #258)
IPRW(d-C)RKCP-NH2, wherein the d-cysteine at position 5 is bound to the cysteine at position 8 via a disulfide bond to form a cyclic peptide (SEQ ID NO.: 155, JM #259)
wherein d- in front of an amino acid indicates a D-amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol on the preceding amino acid, Ac indicates a substitution of an amino group by an acetyl group, Lau indicates lauric acid on the preceding amino acid, Glu-Lau indicates a lauric acid on the preceding amino acid with a glutamate linker, Glu-Ole indicates an oleic acid on the preceding amino acid with a glutamate linker, C16diacid indicates a saturated C16 fatty diacid on the preceding amino acid, Glu-C16diacid indicates a saturated C16 fatty diacid on the preceding amino acid with a glutamate linker, C18diacid indicates a saturated C18 fatty diacid on the preceding amino acid, Glu-C18diacid indicates a saturated C18 fatty diacid on the preceding amino acid with a glutamate linker, OEG-OEG-γGlu-C18diacid indicates a saturated C18 fatty diacid on the preceding amino acid with an OEG-OEG-gamma glutamate linker, OEG represents the residue of 8-amino-3,6-dioxaoctanoic acid, Ara indicates a saturated C20 fatty acid on the preceding amino acid, and Glu-Ara indicates a saturated C20 fatty acid on the preceding amino acid with a glutamate linker.

The inventive peptide derivatives are about 100 times more effective in binding to CXCR4 than previously known peptides. Furthermore, the invention provides peptides with a significantly higher plasma stability than peptides of the state of the art. Furthermore, the invention provides peptides with a significantly higher in vivo circulation half-life than peptides of the state of the art. Consequently, the invention provides peptides with a high therapeutic potential compared to drugs of the state of the art, because smaller doses will be sufficient in order to provide the desired effect.

The term derivative means all length fragments of the peptide EPI-X4 (SEQ ID NO.: 1) including truncations at the N and C terminus, the peptide of the invention containing amino acid residue substitutions including D-amino acid residues and modified amino acid residues as well as peptides containing disulfide bonds and extension at the N and C terminus. Here, the terms peptides, peptide derivatives and derivatives are used synonymously. The term peptides does also extent to cyclized peptides, if the inventive peptides can be provided in cyclized form.

Apart from the modifications mentioned above, the peptides are suitable to be coupled to proteins. Proteins to be coupled to the peptides are, for example, antibodies or human serum albumin (HSA).

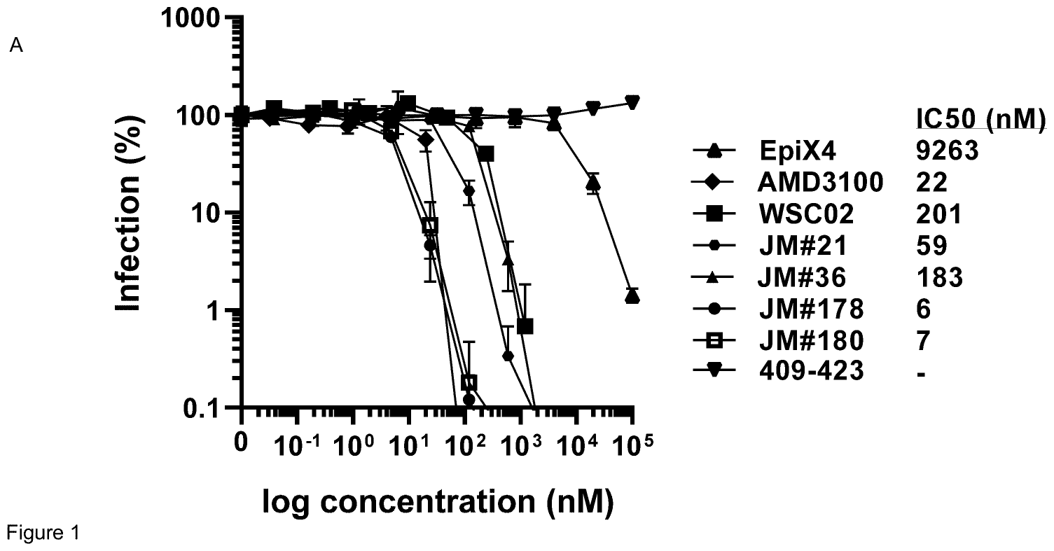

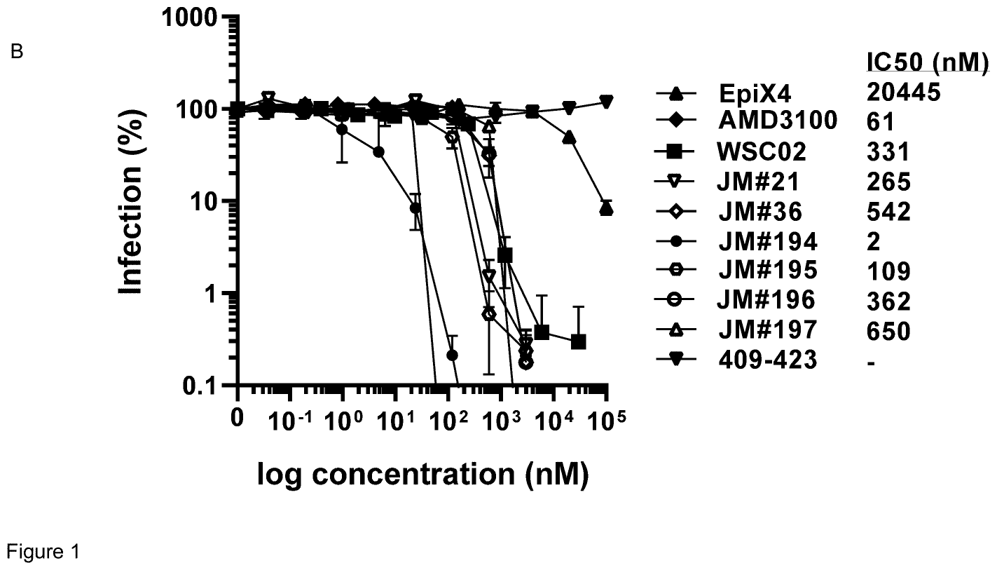

The activity of the peptides was estimated by different assays. First, the activity was tested by an HIV-1 inhibition assay (FIG. 1). The potency of inhibition of CXCR4-tropic X4-HIV-1 indirectly reflects binding affinity of derivatives to CXCR4, as X4-HIV-1 uses CXCR4 together with CD4 for entry into the cell. For cell entry the HIV-1 glycoprotein gp120 needs to bind to the receptors what subsequently leads to cells fusion. In case CXCR4 is blocked by a receptor-ligand, HIV-1 entry is blocked and infection is inhibited.

All tested EPI-X4 derivatives dose dependently and specifically inhibited infection of reporter cells by X4-HIV-1. Peptides EPI-X4 (SEQ ID NO.: 1) and WSC02 (SEQ ID NO.: 2) (EP 3 007 717 B1) were used as reference peptides. In FIG. 1, results of two assays are shown by way of diagrams, wherein the percentage of infected cells depends on the log concentration of different tested peptides (FIGS. 1A and 1B). Using mean values estimated over several tests, JM #21 (SEQ ID NO.: 23) led to an almost 100 fold more effective inhibition (IC₅₀=98 nM) than EPI-X4 (SEQ ID NO.: 1, IC₅₀=8630 nM) and WSC02 (SEQ ID NO.: 2, IC₅₀=310 nM). Peptide derivatives coupled to fatty acids, e.g. palmitic acid (SEQ ID NO.: 75, JM #178; SEQ ID NO.: 77, JM #180; SEQ ID NO.: 91, JM #194; SEQ ID NO.: 92, JM #195; SEQ ID NO.: 93, JM #196; SEQ ID NO.: 94, JM #197) strongly increased anti-HIV-1 activity. Potency of JM #21 (SEQ ID NO.: 23) coupled to palmitic acid was increased by more than 60-fold (IC₅₀ of JM #143 (SEQ ID NO.: 54)=5 nM, not shown) compared to WSC02 (SEQ ID NO.: 2) and C-terminal truncations and amidation of those derivatives even further increased anti-HIV-1 activity (JM #192 (SEQ ID NO.: 89)=2 nM, not shown, JM #194 (SEQ ID NO.: 91)=2 nM). Experiments were done in triplicates. The error bars refer to the standard deviation.

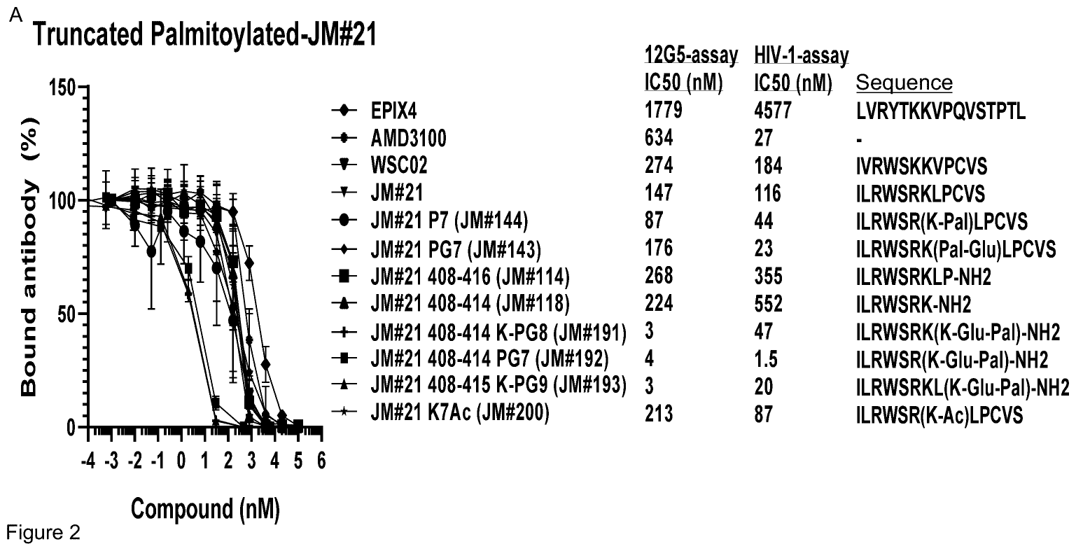

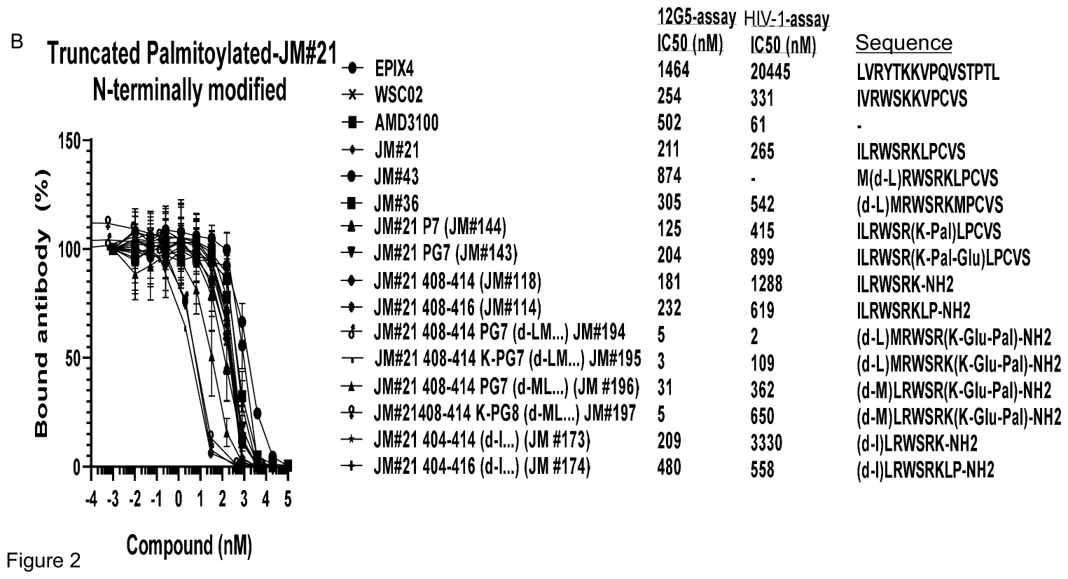

Second, the activity was tested by an antibody competition assay (FIG. 2). Values determined by the competition assay represent the potency of a compound to compete with an antibody that specifically binds to the binding pocket (ECL2) of CXCR4. Those values most probable correlate with CXCR4 binding affinity of the compounds to CXCR4. In FIG. 2 (A and B), results of two assays are shown by way of diagrams, wherein the percentage of bound antibody depends on the concentration of different peptides. To the right of the diagrams, an assignment of the curves to the tested peptides is explained. To the right of the legend a table shows the values of the IC50 in the competition assay (12G5-assay), in a HIV-1-assay and the individual sequences of the tested peptides. All tested EPI-X4 derivatives compete with binding of the antibody. JM #21 (SEQ ID NO.: 23) led to a more effective antibody competition (lower IC₅₀s) than WSC02 (SEQ ID NO.: 2) (IC₅₀=350 nM) and AMD3100 (IC₅₀=690 nM). Taking JM #21 (SEQ ID NO.: 23) as a lead for further development, the inventors designed derivatives that bind with a similar affinity to CXCR4 as JM #21 (SEQ ID NO.: 23), however have molecular weight below 1000 Da (e.g. JM #118 (SEQ ID NO.: 50)). Strikingly, coupling of fatty acids to those shorter derivatives strongly increased binding to CXCR4. Compared to EPI-X4 (SEQ ID NO.: 1) (IC₅₀˜2500 nM) some of the derivatives bind more than 2000-fold more effectively to the receptor (e.g. IC₅₀ of: JM #167 (SEQ ID NO.: 64)=2 nM; JM #178 (SEQ ID NO.: 75)=2 nM; JM #191 (SEQ ID NO.: 88)=1 nM) what is about 300-fold more effective than WSC02 (SEQ ID NO.: 2). Three independent experiments were carried out for each peptide in each assay. The error bars refer to the standard deviation.

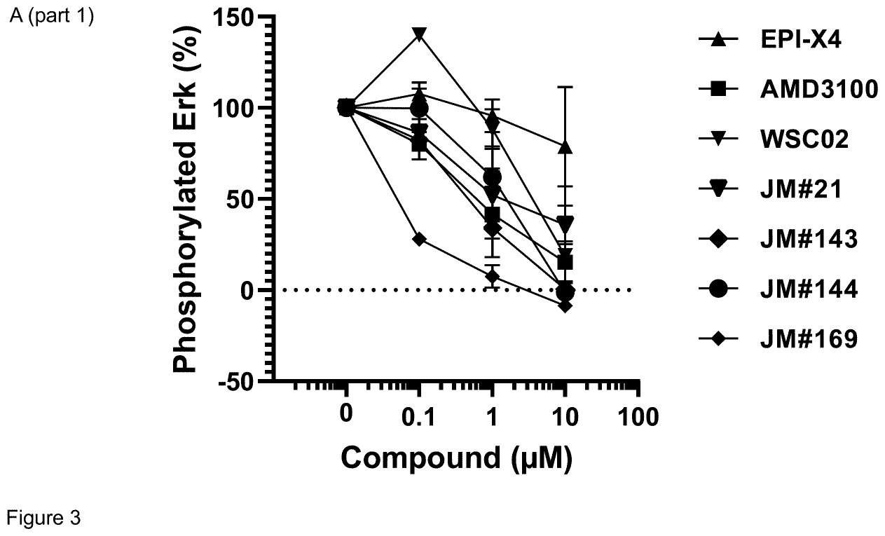

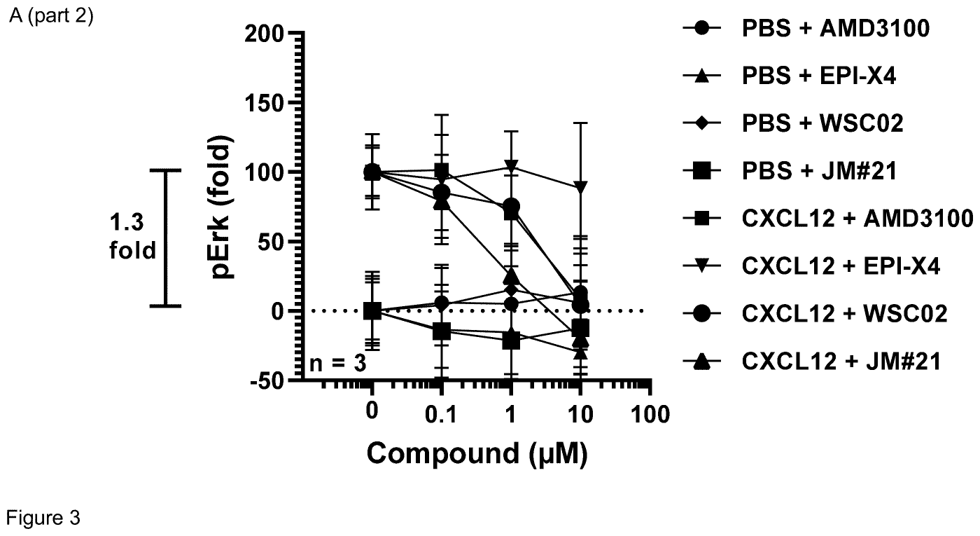

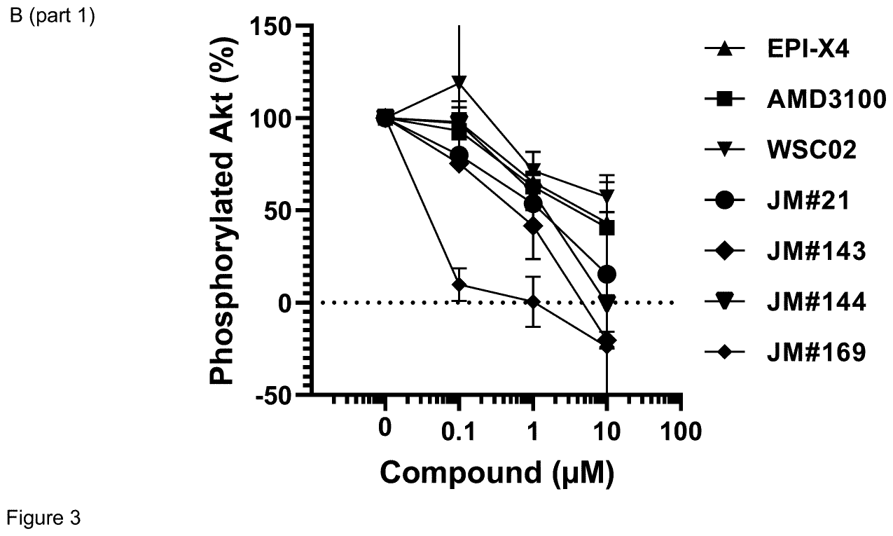

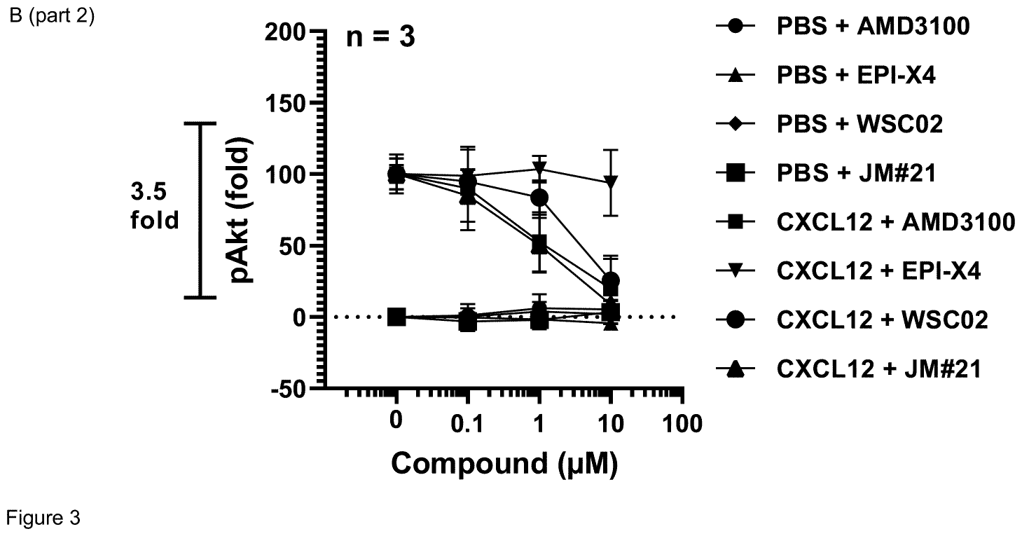

Third, the activity of the claimed EPI-X4 derivatives was tested by the effect on CXCL12/CXCR4-mediated ERK (Extracellular-signal Regulated Kinases) and AKT (a serine/threonine-protein kinase) signaling (FIG. 3). In that assay CXCR4 expressing cells are stimulated with the CXCR4 chemokine-ligand CXCL12, which subsequently leads to phosphorylation of ERK and AKT. Preincubation of cells with CXCR4-antagonists blocks those pathways. In FIG. 3, the results of two assays are shown by way of diagrams, wherein the percentage of phosphorylated ERK (FIG. 3A) and of phosphorylated AKT (FIG. 3B) depends on the concentration of different peptides. All tested EPI-X4 derivatives blocked CXCL12 induced signaling in a dose dependent manner. JM #21 (SEQ ID NO.: 23) blocked signaling more effectively than WSC02. 10 μM WSC02 (SEQ ID NO.: 2) reduced AKT signaling by about 50%, JM #21 (SEQ ID NO.: 23) led to an about 85% inhibition with the same concentration. At a concentration of 1 μM JM #21 blocked CXCL12-induced AKT signaling by about 50% what is about 10-fold as effective as WSC02 (SEQ ID NO.: 2). Also ERK signaling was effectively blocked by JM #21 (SEQ ID NO.: 23) (60% reduction at a concentration of 1 μM), whereas WSC02 (SEQ ID NO.: 2) only reduced Erk phosphorylation by 20% at the same concentration. JM #21 (SEQ ID NO.: 23) was also more effective than AMD3100 (reduction of AKT phosphorylation at 10 μM by about 60%) and EPI-X4 (SEQ ID NO.: 1) (reduction at 10 μM by about 40%). Also JM #18 (SEQ ID NO.: 20) had a very strong antagonistic effect in this assay. JM #18 (SEQ ID NO.: 20) blocked CXCL12-induced AKT and ERK signaling by about 40% at a concentration of 0.1 μM and by almost 70% at a concentration of 1 μM (not shown). Interestingly, palmitic acid coupling led to an increased antagonistic effect. At a concentration of 10 μM all tested palmitic acid coupled derivatives blocked CXCL12 induced AKT and ERK signaling by 100%. JM #143 (SEQ ID NO.: 54), a fatty acid coupled derivative of JM #21 (SEQ ID NO.: 23), blocked AKT and ERK phosphorylation at a concentration of 1 μM by almost 70% and even at a concentration of 0.1 μM signaling was reduced by 20-25%. Strikingly, palmitic acid coupled WSC02 (JM #169, SEQ ID NO.: 66) had a very strong antagonistic activity and reduced AKT signaling by 85% and ERK signaling by over 70% at a concentration of already 0.1 μM, at a concentration of 1 μM AKT signaling was completely and ERK signaling almost completely blocked. Two or three independent experiments were carried out in triplicates for each peptide in each assay. The error bars refer to the standard deviation.

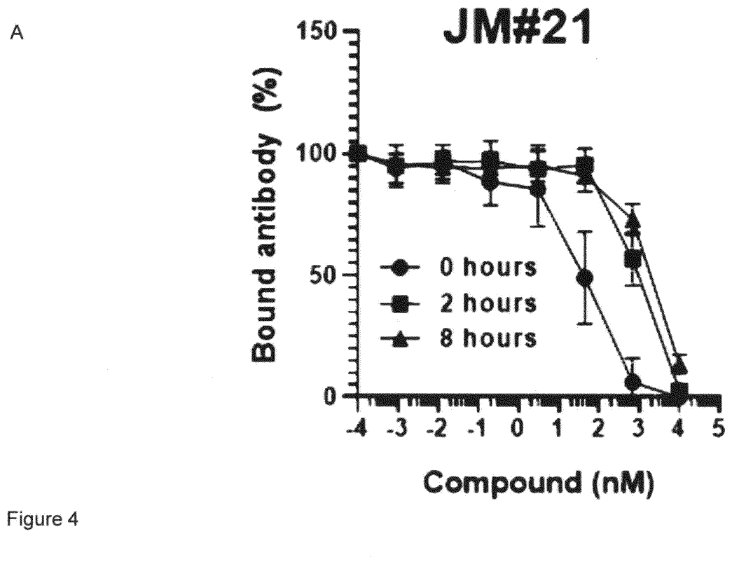

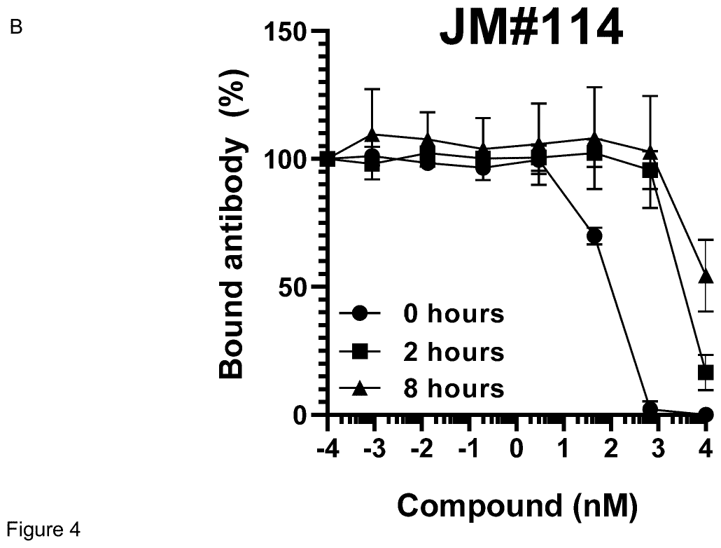

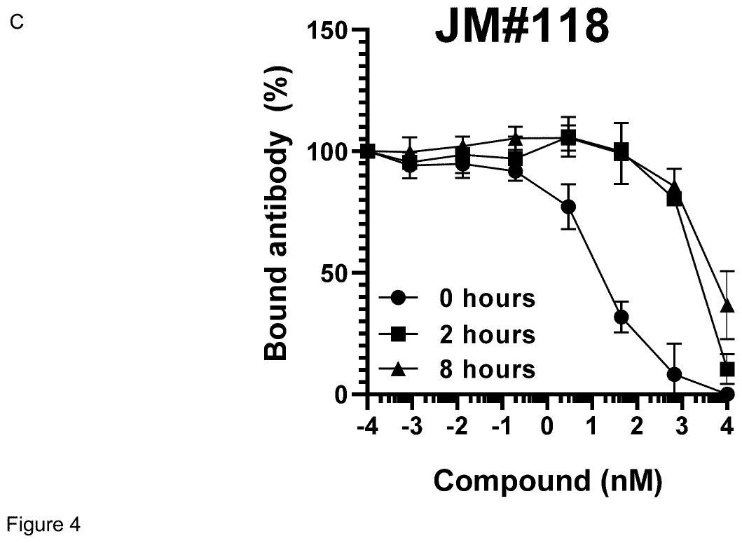

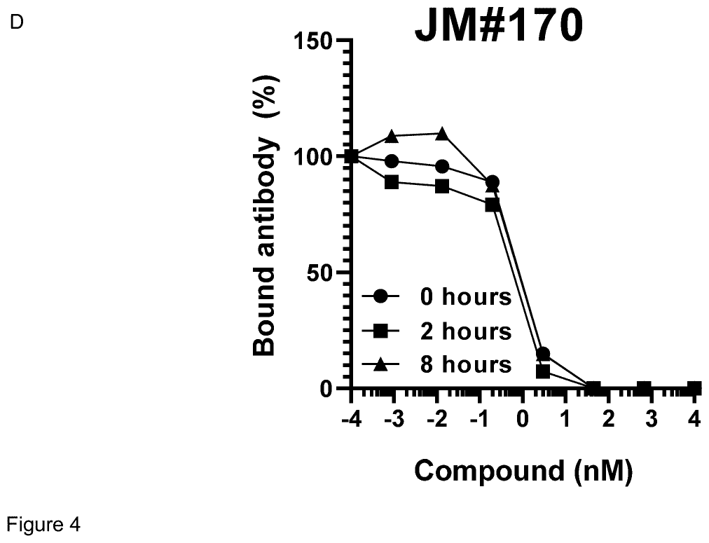

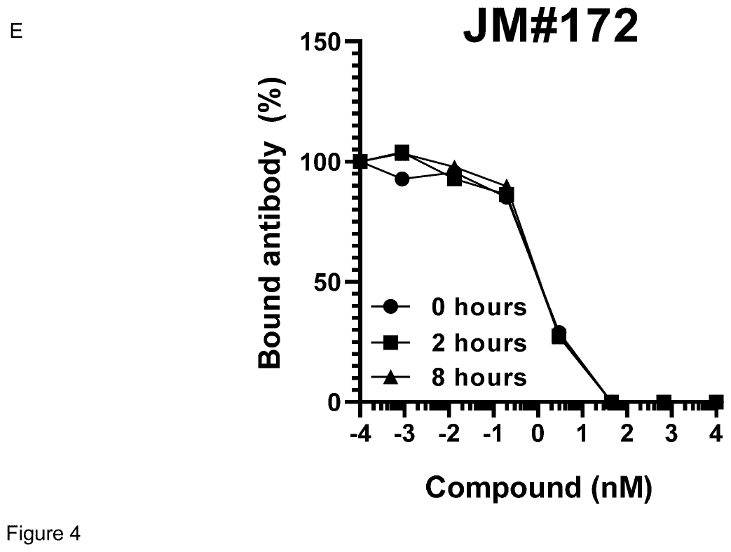

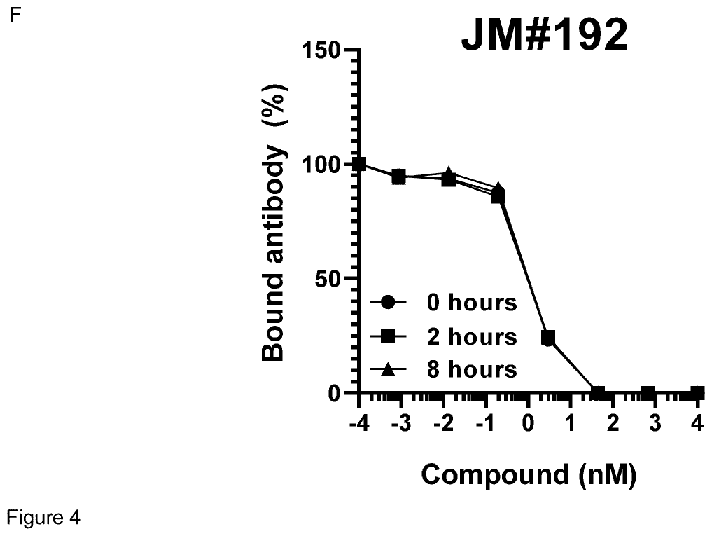

Fourth, the stability of peptides in human plasma and whole human blood was tested (FIG. 4). The functional stability of EPI-X4 (SEQ ID NO.: 1) derivatives was screened using a CXCR4 antibody-based screening approach. Peptides were diluted in full human plasma or blood (>99%) and the functional activity was then measured after either 2 hours or 8 hours and compared to a sample that was not incubated in plasma or blood.

In the diagrams of FIG. 4, the stability of the peptides is shown by way of the percentage of bound antibody depending on the molar concentration of the peptides. The functional stability of both WSC02 (SEQ ID NO.: 2) and JM #21 (SEQ ID NO.: 23) was very low compared to EPI-X4 (SEQ ID NO.: 1) (t ½=17 min) (FIG. 4A). After 2 hours both optimized peptides were completely inactive. Rapid degradation of those variants was also confirmed using mass spectrometry. Also, the truncated variants of both (JM #114 (SEQ ID NO.: 49), FIG. 4B, and JM #118 (SEQ ID NO.: 50), FIG. 4C) were rapidly inactivated in plasma. Using mass spectrometry, the inventors showed that enzymatic degradation of EPI-X4 (SEQ ID NO.: 1) derivatives was exclusively detected at the N-terminus. EPI-X4 (SEQ ID NO.: 1) variants that were modified at the N-terminus (JM #25 (SEQ ID NO.: 27)-JM #44 (SEQ ID NO.: 46)) had a strongly increased functional plasma stability compared to WSC02 (SEQ ID NO.: 2). Whereas the anti-CXCR4 activity was not or not strongly affected (IC50 HIV-1 inhibition assay for: JM #28 (SEQ ID NO.: 30)=246 nM; JM #36 (SEQ ID NO.: 38)=206 nM; JM #43 (SEQ ID NO.: 45)=810 nM) activity was still remained after 2 and even 8 hours of plasma incubation (remaining activity of: JM #28 (SEQ ID NO.: 30)=96% after 2 hours and 77% after 8 hours; JM #36 (SEQ ID NO.: 38)=85% after 2 hours and 81% after 8 hours; JM #43 (SEQ ID NO.: 45)=100% after 2 and 8 hours). Further tests revealed that the peptides JM #173 (SEQ ID NO.: 70) and JM #174 (SEQ ID NO.: 71) were completely stable in human plasma as they retained 100% of their initial activity after 8 hours of plasma incubation (not shown). This was a surprising finding given that their counterpart peptides JM #114 (SEQ ID NO.: 49) and JM #118 (SEQ ID NO.: 50), which are not N-terminally modified with a d-amino acid, were rapidly inactivated in plasma (FIG. 4B, 4C). Due to their strongly reduced size and high stability, JM #173 (SEQ ID NO.: 70) and JM #174 (SEQ ID NO.: 71) may be suitable for enteral administration.

The inventors further aimed to increase plasma stability (and also bioavailability) and therefore designed EPI-X4 (SEQ ID NO.: 1) derivatives that are coupled to fatty acids (e.g. palmitic acid). Most of the fatty acid coupled derivatives had a strongly increased plasma stability as shown e.g. for fatty acid coupled JM #21 (JM #143 (SEQ ID NO.: 54)-JM #145 (SEQ ID NO.: 56)) that did not loose activity at all after 8 hours of plasma incubation. Strikingly, the same is also true for most truncated and fatty acid coupled versions of JM #21 (SEQ ID NO.: 23), WSC02 (SEQ ID NO.: 2) or similar (e.g. JM #170 (SEQ ID NO.: 67), FIG. 4D-JM #172 (SEQ ID NO.: 69), FIG. 4E and JM #191 (SEQ ID NO.: 88)-JM #193 (SEQ ID NO.: 90) (FIG. 4F for JM #192)). The same is true for those variants which were functional stable in the stability assay if combined with the N-terminal modifications (e.g. JM #194 (SEQ ID NO.: 91)-JM #197 (SEQ ID NO.: 94)).

An alternative approach of stabilization was the PEGylation of JM #21 (SEQ ID NO.: 23). PEGylation only marginally or not at all affected anti-CXCR4 activity of the variants (IC50 in HIV-1 inhibition assay for: SC024 (20 kDa)=118 nM, SC029 (telechelic peptide conjugate, 20 kDa)=98 nM, SC033 (5 kDa)=716 nM), however, strongly increased functional plasma stability (remaining activity after 8 hours of plasma incubation for: SC024=30%, SC029=38%, SC033=72%). In FIG. 4A-C, three individual experiments were carried out. The error bars refer to the standard deviation. FIG. 4 D-F are based on one representative experiment.

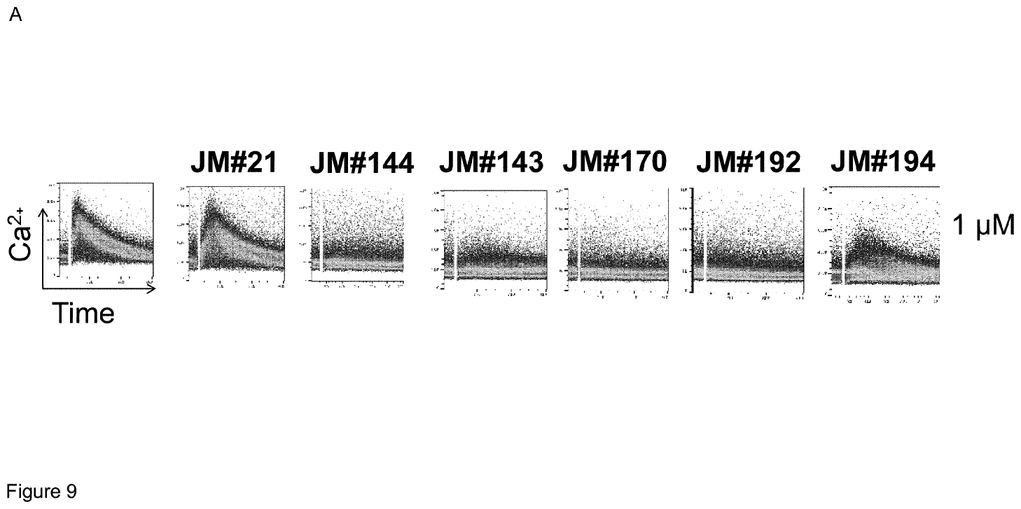

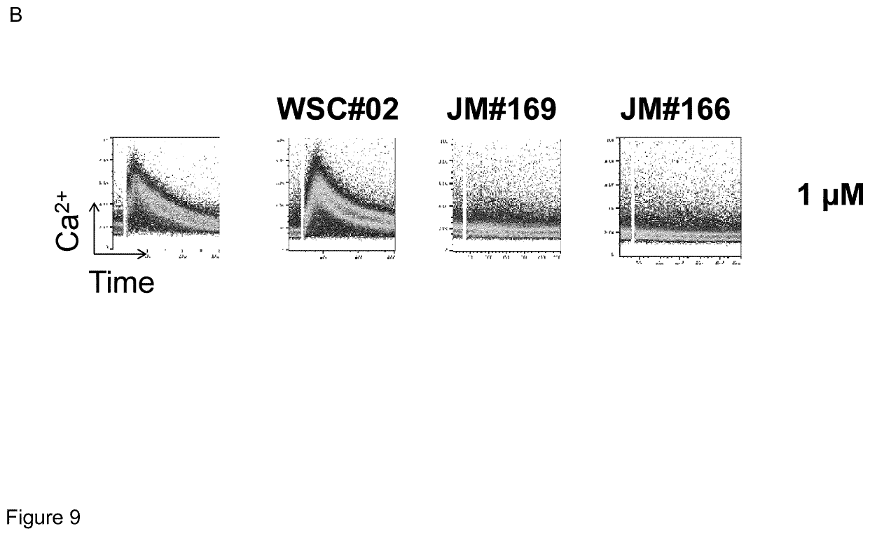

Fifth, it was shown that the peptide derivatives inhibit calcium-signaling. CXCL12 stimulation of CXCR4 expressing B-cells leads to a strong calcium-release. In the presence of CXCR4 antagonists this response is reduced. The inventors saw a reduction of cytokine induced calcium-signaling for 1 μM of JM #21 (SEQ ID NO.: 23) that was much stronger than the reduction seen for WSC02 (SEQ ID NO.: 2) at the same concentration. Fatty acid coupled JM #21 (SEQ ID NO.: 23) variants (JM #143 (SEQ ID NO.: 54), JM #144 (SEQ ID NO.: 55), JM #170 (SEQ ID NO.: 67), JM #192 (SEQ ID NO.: 89), JM #194 (SEQ ID NO.: 91)) (FIG. 9A) as well as fatty acid coupled WSC02 (SEQ ID NO.: 2) variants (FIG. 9B) almost completely blocked calcium-signaling at a concentration of 1 μM. For each peptide, FIG. 9 shows the result of one representative experiment.

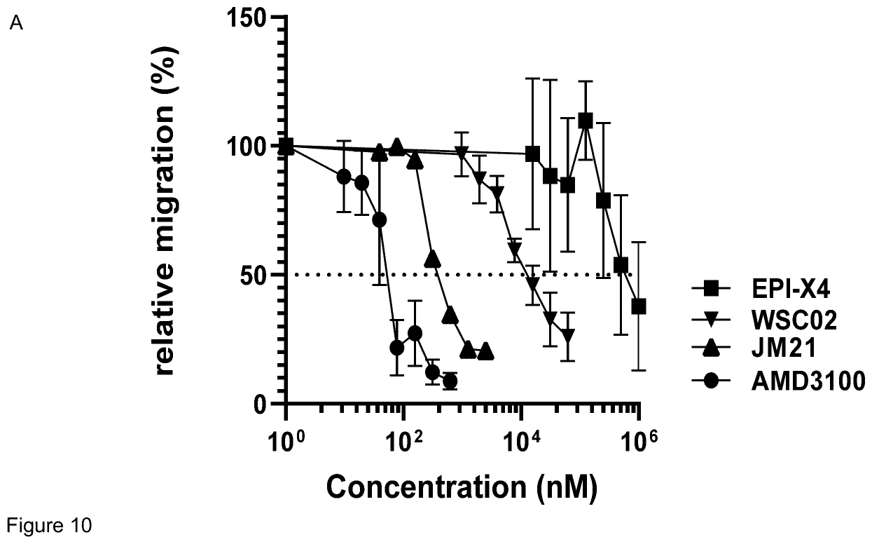

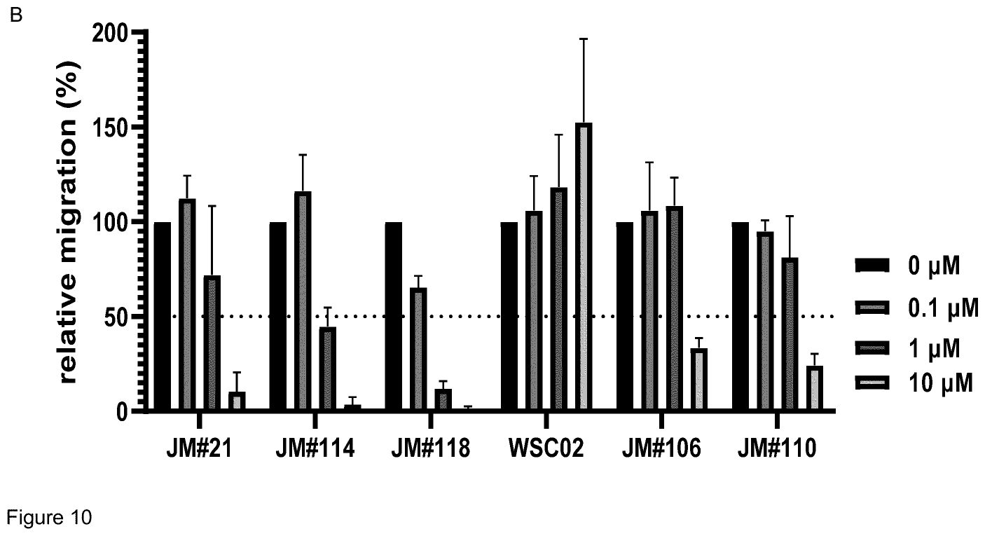

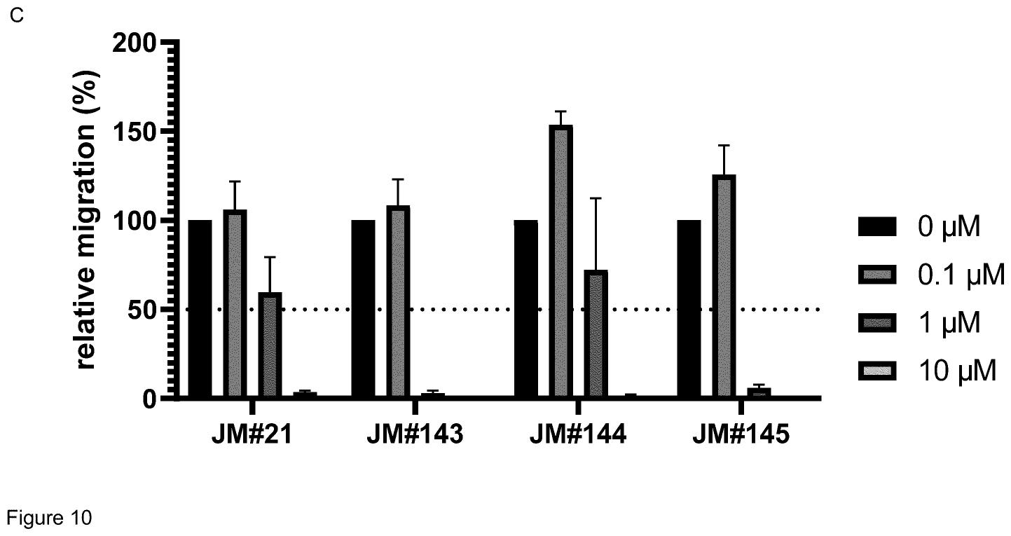

Sixth, all tested EPI-X4 derivatives inhibited CXCL12 induced migration of T-cells. JM #21 (SEQ ID NO.: 23) was more effective than WSC02 (SEQ ID NO.: 2) and EPI-X4 (SEQ ID NO.: 1) (FIG. 10A). Truncated versions of JM #21 (SEQ ID NO.: 23) (JM #114 (SEQ ID NO.: 49), JM #118 (SEQ ID NO.: 50)) were even more effective than the full-length peptide (FIG. 10B). This effect could also be shown for truncated versions of WSC02 (SEQ ID NO.: 2) (JM #106 (SEQ ID NO.: 47), JM #110 (SEQ ID NO.: 48)). Tested fatty acid coupled variants of JM #21 (SEQ ID NO.: 23) (especially JM #143 (SEQ ID NO.: 54)) had a strongly increased antagonistic effect in CXCL12 induced cell migration (FIG. 10C). For each peptide, three independent experiments were performed. The error bars refer to the standard deviation. Further tests revealed a high activity of JM #192 (SEQ ID NO.: 89) and JM #194 (SEQ ID NO.: 91) in inhibiting CXCL12 induced migration of T-cells (not shown). It was further found that peptide conjugation to longer fatty acids seems to be beneficial for blocking cancer cell migration. In particular, all stearic acid variants tested were unexpectedly active. For example, the presence of 30 nM peptide JM #255 (SEQ ID NO.: 150) already led to an almost complete inhibition of cell migration (not shown).

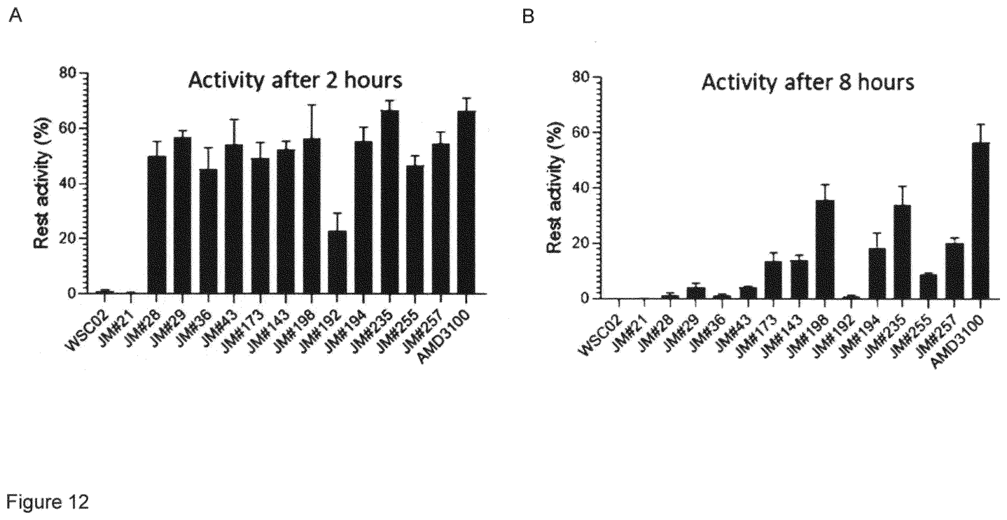

Further, the stability of peptides in human S9 liver fractions (in the presence of cofactors) was tested (FIG. 12). While degradation in blood is the major route of elimination for most peptide therapeutics, hepatic clearance might play a role, especially for the more lipophilic fatty acid conjugates. To simulate and predict hepatic metabolic clearance, peptides were added to human S9 liver fractions. Remaining peptide activity was determined after 2 and 8 hours by antibody competition assay and IC₅₀ values normalized to sample without incubation (t=0). The tested peptides included N-terminally modified variants (JM #28 (SEQ ID NO.: 30), JM #29 (SEQ ID NO.: 31), JM #36 (SEQ ID NO.: 38), JM #43 (SEQ ID NO.: 45), JM #173 (SEQ ID NO.: 70)), the fatty acid conjugated JM #21 (SEQ ID NO.: 23) variants JM #143 (SEQ ID NO.: 54) (C16) and JM #198 (SEQ ID NO.: 95) (C18), and truncated fatty acid conjugated derivatives (JM #192 (SEQ ID NO.: 89), JM #194 (SEQ ID NO.: 91), JM #235 (SEQ ID NO.: 130), JM #255 (SEQ ID NO.: 150), JM #257 (SEQ ID NO.: 152)). All tested derivatives are characterized by their comparably high antagonistic activity and excellent stability in human plasma. Both WSC02 (SEQ ID NO.: 2) and JM #21 (SEQ ID NO.: 23) were readily inactivated by liver enzymes after 2 hours, as expected (FIG. 12A). All stabilized peptides tested retained about 60% of their initial activity after 2 hours with the exception of derivative JM #192 (SEQ ID NO.: 89), which showed faster inactivation (FIG. 12A). After 8 hours of incubation, all non-fatty acid variants were almost completely inactive with the exception of JM #173 (SEQ ID NO.: 70) with about 14% remaining activity (FIG. 12B). Notably, JM #173 (SEQ ID NO.: 70) was completely resistant to plasma enzymes. For fatty acid conjugates, the stearic acid coupled JM #198 (SEQ ID NO.: 95) was more stable than its palmitoylated counterpart JM #143 (SEQ ID NO.: 54) (FIG. 12B). In addition, N-terminal modifications appear to have a positive impact on enzymatic stability. Variants JM #192 (SEQ ID NO.: 89) and JM #255 (SEQ ID NO.: 150) have an unmodified N-terminus and were more rapidly degraded compared to their N-terminally modified variants (JM #194 (SEQ ID NO.: 91), JM #235 (SEQ ID NO.: 130) and JM #257 (SEQ ID NO.: 152), respectively) (FIG. 12B). For each peptide, three independent experiments were performed. The error bars refer to the standard deviation.

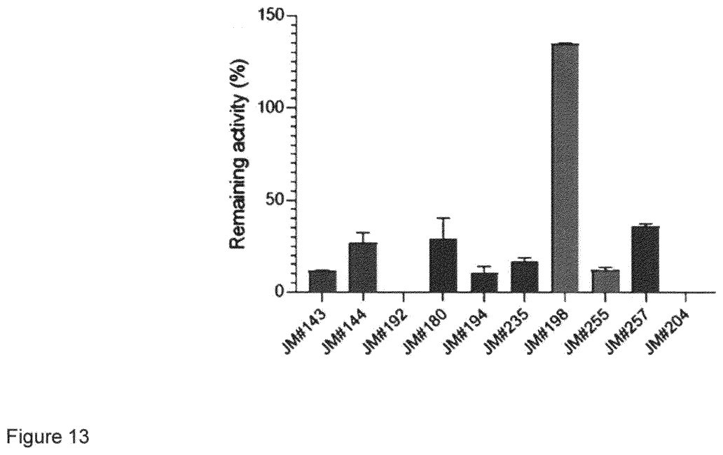

Further, the in vivo stability of peptides was tested (FIG. 13). Peptides were injected into the tail vein of mice. 4 hours post injection, mice were sacrificed and blood taken by heart punctation. Plasma was obtained by centrifugation and tested for remaining activity in antibody competition assay. As control, peptide was spiked into native plasma (ex vivo). IC₅₀ values were determined by non-linear regression assuming 1.8 ml in vivo blood volume. Remaining activity was determined by IC₅₀ (ex vivo)/IC₅₀ (in vivo)×100. The tested peptides included JM #143 (SEQ ID NO.: 54), JM #144 (SEQ ID NO.: 55), JM #192 (SEQ ID NO.: 89), JM #180 (SEQ ID NO.: 77), JM #194 (SEQ ID NO.: 91), JM #235 (SEQ ID NO.: 130), JM #198 (SEQ ID NO.: 95), JM #255 (SEQ ID NO.: 150), JM #257 (SEQ ID NO.: 152) and JM #204 (SEQ ID NO.: 99). Surprisingly, activity of the palmitoylated 7-mer JM #192 (SEQ ID NO.: 89) was completely lost after 4 hours. In contrast, N-terminal modifications (JM #180 (SEQ ID NO.: 77), JM #194 (SEQ ID NO.: 91), JM #235 (SEQ ID NO.: 130)) appear to protect the peptide from elimination or enzymatic inactivation. For JM #180 (SEQ ID NO.: 77) and JM #194 (SEQ ID NO.: 91) about 9% of the conjugates remained active in plasma even 8 hours post injection (not shown). The highest bioavailability after 4 hours was determined for variants JM #144 (SEQ ID NO.: 55) and JM #180 (SEQ ID NO.: 77) that lack the glutamic acid linker (27% and 29% remaining activity, respectively). The length of the conjugated fatty acid has an impact on in vivo stability and bioavailability. Stearic acid (C18) conjugated JM #198 (SEQ ID NO.: 95) remained completely active and available in blood plasma 4 hours post injection. In contrast, activity of the myristoylated (C14) derivative JM #204 (SEQ ID NO.: 99) was lost after this time. Derivative JM #198 (SEQ ID NO.: 95) retained 33% of its activity 8 hours post injection and was finally completely eliminated after 24 hours (not shown). Even though stabilizing properties of stearic acid were not completely transferred to the shorter versions JM #255 (SEQ ID NO.: 150) and JM #257 (SEQ ID NO.: 152), C18 conjugates are excellent peptide derivatives. JM #198 (SEQ ID NO.: 95) is characterized by high enzymatic resistance, comparably long circulation half-lives and excellent antagonistic activities. Shown are data from 2 individual mice measured in duplicates. The error bars refer to the standard deviation.

A toxicity test in Zebrafish showed that all tested derivatives were not toxic for Zebrafish embryos at their respective active concentrations.

The peptides of group 1 are characterized by an inhibitory activity characterized by a half maximal inhibitory concentration (IC₅₀) of below 5 nM, as measured in an X4-HIV-1 inhibition assay (in order to estimate the capability of blocking X4-HIV-1 infection). The X4-HIV inhibition assay is designed to measure the activity of the inventive peptides by their efficiency of blocking the infection of tissue culture cells by CXCR4-tropic HIV-1 variants.

The peptides of group 2 are characterized by an inhibitory activity characterized by an IC₅₀ between 5 and 10 nM, as measured in an HIV inhibition assay.

The peptides of group 3 are characterized by an inhibitory activity characterized by an IC₅₀ between 10 and 50 nM, as measured in an HIV inhibition assay.

The peptides of group 4 are characterized by an inhibitory activity characterized by an IC₅₀ between 50 and 150 nM, as measured in an HIV inhibition assay.

The peptides of group 5 are characterized by an inhibitory activity characterized by an IC₅₀ of above 150 nM, as measured in an HIV inhibition assay.

The peptides of group 6 are characterized by an IC₅₀ below 25 nM, as measured in an antibody competition assay. These peptides had an IC₅₀ of above 150 nM, as measured in an HIV inhibition assay.

The peptides of group 7 are characterized by a relative activity of 100%, as measured after 8 hours of plasma incubation. In this test, the peptides were incubated in human plasma for a certain time period. The relative activity is estimated by measuring the maintenance of the capability of blocking X4-HIV-1 infection over the certain time period or by measuring the activity by the 12G5 antibody competition assay over the certain time period.

The peptides of group 8 are characterized by a relative activity of 100% after 2 hours of plasma incubation, but less (75-99%) after 8 hours of plasma incubation.

The peptides of group 9 are characterized by a relative activity of 70-99% after 2 hours of plasma incubation.

The peptides of group 10 are characterized by both an IC₅₀ below 50 nM and a relative activity of 100% after 8 hours of plasma incubation. With other words, these peptides were shown to maintain a high activity over a relatively long period of time.

The peptides of group 11 are cyclized peptides. These peptides are particularly suitable for oral delivery (oral administration) to a subject such as a patient. Cyclization leads to increased stability of the peptides against protease-mediated degradation and shields positively charged amino acid residues.

The term cyclized peptide (or cyclic peptide) as used herein refers to a peptide having a circular sequence of bonds. This can be through a connection between the amino end and the carboxyl end of the peptide, a connection between the amino end and a side chain of the peptide, a connection between the carboxyl end and a side chain of the peptide, or a connection between two side chains of the peptide.

The term thioester bond is used synonymously to the term thiolester bond.

The inventors have further synthesized the following linear equivalents of the cyclized peptides of group 11:

(SEQ ID NO.: 110, JM#218 linear)
ILRWCRKPC-NH2
(SEQ ID NO.: 112, JM#219 linear)
ILRW(d-C)RKPC-NH2
(SEQ ID NO.: 114, JM#220 linear)
IPRW(d-C)RKC-NH2
(SEQ ID NO.: 123, JM#231 linear)
IPRW(d-C)RKP
(SEQ ID NO.: 125, JM#232 linear)
ILRW(d-C)RKP
(SEQ ID NO.: 127, JM#233 linear)
IPRW(d-S)RKP
(SEQ ID NO.: 129, JM#234 linear)
ILRW(d-S)RKP
(SEQ ID NO.: 154, JM#258 linear)
IMRWCRKPC-NH2
(SEQ ID NO.: 156, JM#259 linear)
IPRW(d-C)RKCP-NH2

The linear equivalents are used as control peptides in assays for characterizing the cyclized peptides such as activity and stability assays.

Substitution of the N-terminal amino group, introduction of D-amino acids as well as certain amino acid substitutions at the N-terminus of the peptide have been shown to inhibit protease activity. These kinds of modification are advantageous because they increase the plasma stability of the peptides, as indicated by a half-life of up to 29 hours.

The coupling of fatty acids, i.e. of palmitic acid, decanoic acid, myristic acid, oleic acid, and stearic acid has been shown to provide higher activity of the peptides. Furthermore, the coupling of fatty acids could prolong the circulation of the peptides at a certain level of concentration in vivo. The same applies to the coupling of other fatty acids such as lauric acid, saturated C16 fatty diacid, saturated C18 fatty diacid, and saturated C20 fatty acid.

In the peptide of SEQ ID NO.: 121 (JM #230), the OEG-OEG-γGlu linker (2×OEG-γGlu linker) is used to couple the fatty acid to the peptide. OEG represents the residue of 8-amino-3,6-dioxaoctanoic acid (i.e. a group of the formula —NH—(CH2)2-0-(CH2)2-0-CH2-CO—). The two OEG entities of the linker are consecutively coupled to the side chain of the lysine of the peptide. The fatty acid is coupled to the two OEG entities via the gamma glutamate entity of the linker.

Cholesterol has been shown to increase the relative stability of the peptides in human plasma as well as the biological availability.

A second aspect of the invention is directed to a peptide consisting of one of the following amino acid sequences, selected from any of the groups 1 to 11, wherein
Group 7 consists of

(SEQ ID NO.: 70, JM#173)
d-ILRWSRK-NH2
(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 71, JM#174)
d-ILRWSRKLP-NH2
(SEQ ID NO.: 95, JM#198)
ILRWSRK(Glu-Ste)LPCVS
(SEQ ID NO.: 104, JM#213)
ILRWSRK(Glu-Lau)LPCVS
(SEQ ID NO.: 108, JM#217)
ILRWSRK(Glu-Ole)LPCVS
(SEQ ID NO.: 117, JM#226)
ILRWSRK(C16diacid)LPCVS
(SEQ ID NO.: 118, JM#227)
ILRWSRK(Glu-C16diacid)LPCVS
(SEQ ID NO.: 119, JM#228)
ILRWSRK(C18diacid)LPCVS
(SEQ ID NO.: 120, JM#229)
ILRWSRK(Glu-C18diacid)LPCVS
(SEQ ID NO.: 121, JM#230)
ILRWSRK(OEG-OEG-γGlu-C18diacid)LPCVS
(SEQ ID NO.: 130, JM#235)
d-LLRWSRK(Glu-Pal)-NH2
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 133, JM#238)
d-ILRWSRK(Glu-Pal)-NH2
(SEQ ID NO.: 139, JM#244)
ILRWSRK(C16diacid)-NH2
(SEQ ID NO.: 140, JM#245)
ILRWSRK(C18diacid)-NH2
(SEQ ID NO.: 141, JM#246)
ILRWSRK(Glu-C16diacid)-NH2
(SEQ ID NO.: 142, JM#247)
ILRWSRK(Glu-C18diacid)-NH2
(SEQ ID NO.: 143, JM#248)
d-LLRWSRK(C16diacid)-NH2
(SEQ ID NO.: 144, JM#249)
d-LLRWSRK(C18diacid)-NH2
(SEQ ID NO.: 145, JM#250)
d-LLRWSRK(Glu-C16diacid)-NH2
(SEQ ID NO.: 146, JM#251)
d-LLRWSRK(Glu-C18diacid)-NH2
(SEQ ID NO.: 147, JM#252)
ILRWSRK(Ara)LPCVS
(SEQ ID NO.: 148, JM#253)
ILRWSRK(Glu-Ara)LPCVS
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 151, JM#256)
d-LLRWSRK(Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2
(SEQ ID NO.: 159, JM#262)
ILRWSRK(Myr)-NH2
(SEQ ID NO.: 161, JM#264)
LVRYTKK(Glu-Pal)-NH2
(SEQ ID NO.: 162, JM#265)
d-LVRYTKK(Glu-Pal)-NH2
(SEQ ID NO.: 163)
ILRWSRK(Pal-Glu)LPSVS

Group 1 consists of

(SEQ ID NO.: 20, JM#18)
ILRWSRKMPCLS
(SEQ ID NO.: 55, JM#144)
ILRWSRK(Pal)LPCVS
(SEQ ID NO.: 89, JM#192)
ILRWSRK(Glu-Pal)-NH2
(SEQ ID NO.: 91, JM#194)
d-LMRWSRK(Glu-Pal)-NH2
(SEQ ID NO.: 150, JM#255)
ILRWSRK(Glu-Ste)-NH2

Group 2 consists of

(SEQ ID NO.: 69, JM#172)
ILRWSRK(Pal)L-NH2
(SEQ ID NO.: 75, JM#178)
d-LMRWSRK(Pal)MP-NH2
(SEQ ID NO.: 77, JM#180)
d-LMRWSRK(Pal)-NH2
(SEQ ID NO.: 80, JM#183)
ILRWSRK(Ole)LPCVS
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 152, JM#257)
d-LLRWSRK(Glu-Ste)-NH2

Group 3 consists of

(SEQ ID NO.: 53, JM#141)
d-LLRWSRK(Pal)MPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 66, JM#169)
IVRWSKK(Pal)VPCVS
(SEQ ID NO.: 67, JM#170)
ILRWSRK(Pal)-NH2
(SEQ ID NO.: 68, JM#171)
ILRWSRK(Pal)LP-NH2
(SEQ ID NO.: 88, JM#191)
ILRWSRKK(Glu-Pal)-NH2
(SEQ ID NO.: 90, JM#193)
ILRWSRKLK(Glu-Pal)-NH2
(SEQ ID NO.: 96, JM#199)
ILRWSRKK(Glu-Ste)LPCVS
(SEQ ID NO.: 98, JM#203)
ILRWSRK(Glu-Dec)LPCVS
(SEQ ID NO.: 100, JM#205)
ILRWSRK(Glu-Ste)LPCVS
(SEQ ID NO.: 107, JM#216)
ILRWSRK(Myr)LPCVS
(SEQ ID NO.: 149, JM#254)
ILRWSRK(Ste)-NH2
(SEQ ID NO.: 157, JM#260)
d-LLRWSRK(Glu-Myr)-NH2
(SEQ ID NO.: 160, JM#263)
ILRWSRK(Glu-Myr)-NH2

Group 4 consists of

(SEQ ID NO.: 10, JM#8)
ILRWSRKVPCVS
(SEQ ID NO.: 12, JM#10)
IFRWSRKVPCVS
(SEQ ID NO.: 29, JM#27)
MLRWSRKMPCVS
(SEQ ID NO.: 36, JM#34)
MMRWSRKMPCVS
(SEQ ID NO.: 41, JM#39)
MLRWSRKLPCVS
(SEQ ID NO.: 51, JM#122)
ILRWSRKLPSVS
(SEQ ID NO.: 52, JM#140)
d-LLRWSRK(Glu-Pal)MPCVS
(SEQ ID NO.: 59, JM#149)
ILRWSRK(Pal)MPCLS
(SEQ ID NO.: 92, JM#195)
d-LMRWSRKK(Glu-Pal)-NH2
(SEQ ID NO.: 97, JM#200)
ILRWSRK-AcLPCVS
(SEQ ID NO.: 105, JM#214)
ILRWSRK(Lau)LPCVS
(SEQ ID NO.: 151, JM#256)
d-LLRWSRK(Ste)-NH2
(SEQ ID NO.: 159, JM#262)
ILRWSRK(Myr)-NH2

Group 5 consists of

(SEQ ID NO.: 3, JM#1)
ILRWSKKVPCVS
(SEQ ID NO.: 4, JM#2)
IFRWSKKVPCVS
(SEQ ID NO.: 5, JM#3)
IVRWSRKVPCVS
(SEQ ID NO.: 6, JM#4)
IVRWSHKVPCVS
(SEQ ID NO.: 7, JM#5)
IVRWSKKLPCVS
(SEQ ID NO.: 8, JM#6)
IVRWSKKIPCVS
(SEQ ID NO.: 9, JM#7)
IVRWSKKFPCVS
(SEQ ID NO.: 11, JM#9)
ILRWSHKVPCVS
(SEQ ID NO.: 13, JM#11)
IFRWSHKVPCVS
(SEQ ID NO.: 14, JM#12)
IVRWSKKMPCVS
(SEQ ID NO.: 16, JM#14)
IVRWSKKVPCd-VS
(SEQ ID NO.: 17, JM#15)
ILRWSRKVPCd-VS
(SEQ ID NO.: 18, JM#16)
IIRWSRKMPCVS
(SEQ ID NO.: 25, JM#23)
ILRWSRKVPSVS
(SEQ ID NO.: 26, JM#24)
ILRWSRKMPSVS
(SEQ ID NO.: 27, JM#25)
Ac-SLRWSRKMPCVS
(SEQ ID NO.: 28, JM#26)
d-Ac-SLRWSRKMPCVS
(SEQ ID NO.: 30, JM#28)
d-MLRWSRKMPCVS
(SEQ ID NO.: 31, JM#29)
d-LLRWSRKMPCVS
(SEQ ID NO.: 32, JM#30)
d-FLRWSRKMPCVS
(SEQ ID NO.: 33, JM#31)
GLRWSRKMPCVS
(SEQ ID NO.: 34, JM#32)
Ac-SMRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 39, JM#37)
d-FMRWSRKMPCVS
(SEQ ID NO.: 40, JM#38)
d-GMRWSRKMPCVS
(SEQ ID NO.: 42, JM#40)
d-MLRWSRKLPCVS
(SEQ ID NO.: 43, JM#41)
Id-LRWSRKLPCVS
(SEQ ID NO.: 44, JM#42)
Id-LRWSRKMPCVS
(SEQ ID NO.: 45, JM#43)
Md-LRWSRKLPCVS
(SEQ ID NO.: 46, JM#44)
Md-LRWSRKMPCVS
(SEQ ID NO.: 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS
(SEQ ID NO.: 106, JM#215)
ILRWSRK(Dec)LPCVS
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 158, JM#261)
d-LLRWSRK(Myr)-NH2
(SEQ ID NO.: 163)
ILRWSRK(Pal-Glu)LPSVS
(SEQ ID NO.: 164)
ILRWSRK(Glu-Ste)LPSVS

Group 6 consists of

(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 164)
ILRWSRK(Glu-Ste)LPSVS

Group 8 consists of

(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2

Group 9 consists of

(SEQ ID NO.: 27, JM#25)
Ac-SLRWSRKMPCVS
(SEQ ID NO.: 30, JM#28)
d-MLRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 52, JM#140)
d-LLRWSRK(Glu-Pal)MPCVS
(SEQ ID NO.: 75, JM#178)
d-LMRWSRK(Pal)MP-NH2
(SEQ ID NO.: 106, JM#215)
ILRWSRK(Dec)LPCVS
(SEQ ID NO.: 158, JM#261)
d-LLRWSRK(Myr)-NH2
(SEQ ID NO.: 160, JM#263)
ILRWSRK(Glu-Myr)-NH2

Group 10 consists of

(SEQ ID NO.: 55, JM#144)
ILRWSRK(Pal)LPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 69, JM#172)
ILRWSRK(Pal)L-NH2
(SEQ ID NO.: 77, JM#180)
d-LMRWSRK(Pal)-NH2
(SEQ ID NO.: 88, JM#191)
ILRWSRKK(Glu-Pal)-NH2
(SEQ ID NO.: 89, JM#192)
ILRWSRK(Glu-Pal)-NH2
(SEQ ID NO.: 90, JM#193)
ILRWSRKLK(Glu-Pal)-NH2
(SEQ ID NO.: 91, JM#194)
d-LMRWSRK(Glu-Pal)-NH2
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2

Group 11 consists of
ILRWCRKPC-NH2, wherein the cysteine at position 5 is bound to the cysteine at position 9 via a disulfide bond to form a cyclic peptide (SEQ ID NO.: 109, JM #218)
ILRW(d-C)RKPC-NH2, wherein the d-cysteine at position 5 is bound to the cysteine at position 9 via a disulfide bond to form a cyclic peptide (SEQ ID NO.: 111, JM #219)
IPRW(d-C)RKC-NH2, wherein the d-cysteine at position 5 is bound to the cysteine at position 8 via a disulfide bond to form a cyclic peptide (SEQ ID NO.: 113, JM #220)
ILRWSRKLPCVS, wherein the lysine at position 7 is bound to the carboxyl-terminus via a peptide bond to form a cyclic peptide (SEQ ID NO.: 115, JM #221)
ILRWSKKLPCVS, wherein the lysine at position 6 is bound to the carboxyl-terminus via a peptide bond to form a cyclic peptide (SEQ ID NO.: 116, JM #222)
IPRW(d-C)RKP, wherein the d-cysteine at position 5 is bound to the proline at position 8 via a thioester bond to form a cyclic peptide (SEQ ID NO.: 122, JM #231)
ILRW(d-C)RKP, wherein the d-cysteine at position 5 is bound to the proline at position 8 via a thioester bond to form a cyclic peptide (SEQ ID NO.: 124, JM #232)
IPRW(d-S)RKP, wherein the d-serine at position 5 is bound to the proline at position 8 via an ester bond to form a cyclic peptide (SEQ ID NO.: 126, JM #233)
ILRW(d-S)RKP, wherein the d-serine at position 5 is bound to the proline at position 8 via an ester bond to form a cyclic peptide (SEQ ID NO.: 128, JM #234)
IMRWCRKPC-NH2, wherein the cysteine at position 5 is bound to the cysteine at position 9 via a disulfide bond to form a cyclic peptide (SEQ ID NO.: 153, JM #258)
IPRW(d-C)RKCP-NH2, wherein the d-cysteine at position 5 is bound to the cysteine at position 8 via a disulfide bond to form a cyclic peptide (SEQ ID NO.: 155, JM #259)
wherein d- in front of an amino acid indicates a D-amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol on the preceding amino acid, Ac indicates a substitution of an amino group by an acetyl group, Lau indicates lauric acid on the preceding amino acid, Glu-Lau indicates a lauric acid on the preceding amino acid with a glutamate linker, Glu-Ole indicates an oleic acid on the preceding amino acid with a glutamate linker, C16diacid indicates a saturated C16 fatty diacid on the preceding amino acid, Glu-C16diacid indicates a saturated C16 fatty diacid on the preceding amino acid with a glutamate linker, C18diacid indicates a saturated C18 fatty diacid on the preceding amino acid, Glu-C18diacid indicates a saturated C18 fatty diacid on the preceding amino acid with a glutamate linker, OEG-OEG-γGlu-C18diacid indicates a saturated C18 fatty diacid on the preceding amino acid with an OEG-OEG-gamma glutamate linker, OEG represents the residue of 8-amino-3,6-dioxaoctanoic acid, Ara indicates a saturated C20 fatty acid on the preceding amino acid, and Glu-Ara indicates a saturated C20 fatty acid on the preceding amino acid with a glutamate linker,
and wherein the peptide is conjugated to a complexing agent.

In other words, the second aspect of the invention relates to a conjugate in which the peptide according to the invention is conjugated to a complexing agent.

In a preferred embodiment, the peptide is C-terminally conjugated to the complexing agent. C-terminal conjugation of the complexing agent was shown to have no influence on the activity of the peptides.

In a preferred embodiment, the complexing agent is a chelator such as, for example, dodecane tetraacetic acid (DOTA), deferoxamine. 1,4,7-Triazacyclononane-1,4,7-triacetic acid (NOTA), N, N′-bis-[2-hydroxy (carboxyethyl)benzyl]ethylenediamine-N, N′-diacetic acid (HBED-CC), Triazacyclononane-phosphinic acid (TRAP) or Tris(hydroxypyridinone) (THP).

In a preferred embodiment, the complexing agent is dodecane tetraacetic acid (DOTA) or deferoxamine. The conjugated peptide preferably is labelled with a radioactive nuclide.

DOTA preferably is conjugated to the peptide via a lysine residue. In case the peptide has no available lysine, DOTA is conjugated to the peptide via an additional lysine that is coupled to the C-terminal amino acid of the peptide.

In case the peptide is C-terminally conjugated to DOTA and the peptide has an amino acid other than lysine as C-terminal amino acid, DOTA is conjugated to the peptide via an additional lysine that is coupled to the C-terminal amino acid of the peptide. In case the peptide has lysine as C-terminal amino acid, DOTA is conjugated to the C-terminal lysine of the peptide or DOTA is conjugated to the peptide via an additional lysine that is coupled to the C-terminal lysine of the peptide.

DOTA may alternatively be conjugated to the peptide via a cysteine residue. The above statements regarding the conjugation of DOTA via a lysine residue likewise apply to the conjugation of DOTA via a cysteine residue.

Deferoxamine preferably is conjugated to the peptide via a cysteine residue. In case the peptide has no cysteine, deferoxamine is conjugated to the peptide via an additional cysteine that is coupled to the C-terminal amino acid of the peptide.

In case the peptide is C-terminally conjugated to deferoxamine and the peptide has an amino acid other than cysteine as C-terminal amino acid, deferoxamine is conjugated to the peptide via an additional cysteine that is coupled to the C-terminal amino acid of the peptide. In case the peptide has cysteine as C-terminal amino acid, deferoxamine is conjugated to the C-terminal cysteine of the peptide or deferoxamine is conjugated to the peptide via an additional cysteine that is coupled to the C-terminal cysteine of the peptide.

Deferoxamine may alternatively be conjugated to the peptide via a lysine residue. The above statements regarding the conjugation of deferoxamine via a cysteine residue likewise apply to the conjugation of deferoxamine via a lysine residue.

The above statements regarding the conjugation of DOTA and deferoxamine likewise apply to conjugation of the peptide to a different complexing agent.

In a particularly preferred embodiment, the complexing agent is the chelator DOTA. With other words, the peptide is conjugated to the chelator DOTA, wherein the peptide preferably is C-terminally conjugated to DOTA. DOTA (also known as tetraxetan) is an organic compound with the formula (CH₂CH₂NCH₂CO₂H)₄. The molecule consists of a central 12-membered tetraaza (i.e., containing four nitrogen atoms) ring. The acronym DOTA (for dodecane tetraacetic acid) is shorthand for both the tetracarboxylic acid and its various conjugate bases. DOTA-conjugated peptides are suitable for labelling with radioactive nuclides, e.g. ⁶⁸Ga and ¹⁷⁷Lu. Consequently, these peptides are useful in applications in diagnostic and therapeutic approaches. The inventive EPI-X4 (SEQ ID NO.: 1) derivatives, which are specific for the CXCR4, can be used for blending diagnostic and therapeutic with the same molecule (radiotheranostics). Radiotheranostics based on these peptides is offering new imaging tests and therapeutic options to patients suffering from CXCR4-expressing malignancies.

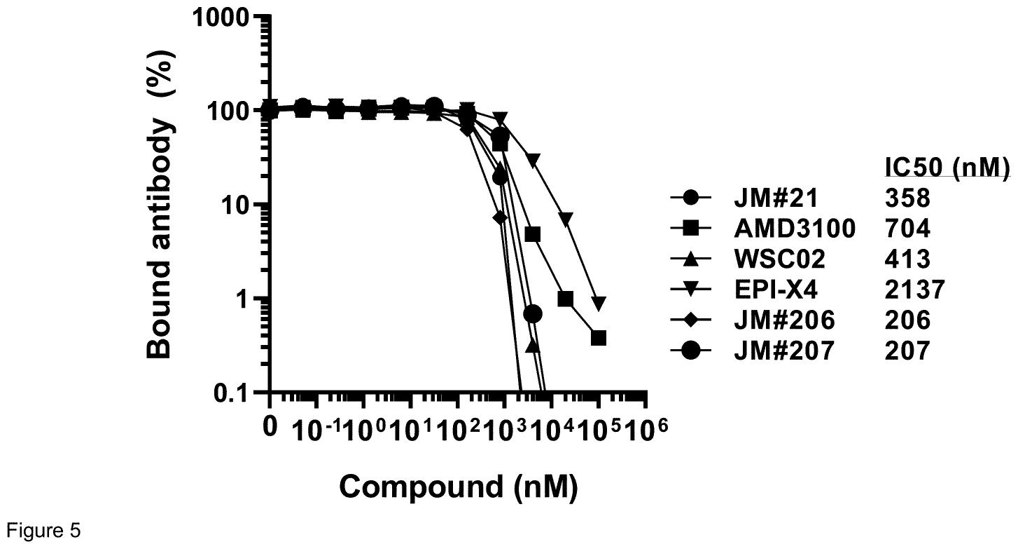

FIG. 5 shows a diagram of an antibody competition assay (based on one representative experiment per peptide), wherein the percentage of bound antibody is depending on the molar concentration of the shown peptides. It is demonstrated that the DOTA-conjugated peptides JM #206 (SEQ ID NO.: 101) (JM #21 (SEQ ID NO.: 23) with DOTA) and JM #207 (SEQ ID NO.: 102) (JM #122 (SEQ ID NO.: 51) with DOTA) displaced the antibody as efficiently as the non-conjugated JM #21 (SEQ ID NO.: 23). Similarly, both DOTA-conjugated peptides suppressed HIV-1 infection with similar potency as the non-conjugated peptide.

The inventors further synthesized the following DOTA-conjugated peptides: the peptide of SEQ ID NO.: 165 (JM #29 (SEQ ID NO.: 31) with DOTA conjugated to the peptide via an additional lysine that is coupled to the C-terminal amino acid of the peptide), SEQ ID NO.: 166 (JM #118 (SEQ ID NO.: 50) with DOTA conjugated to the peptide via the C-terminal lysine of the peptide), SEQ ID NO.: 167 (JM #118 (SEQ ID NO.: 50) with DOTA conjugated to the peptide via an additional lysine that is coupled to the C-terminal amino acid of the peptide), SEQ ID NO.: 168 (JM #173 (SEQ ID NO.: 70) with DOTA conjugated to the peptide via the C-terminal lysine of the peptide), SEQ ID NO.: 169 (JM #173 (SEQ ID NO.: 70) with DOTA conjugated to the peptide via an additional lysine that is coupled to the C-terminal amino acid of the peptide), SEQ ID NO.: 170 (JM #235 (SEQ ID NO.: 130) with DOTA conjugated to the peptide via the C-terminal lysine of the peptide), SEQ ID NO.: 171 (JM #235 (SEQ ID NO.: 130) with DOTA conjugated to the peptide via an additional lysine that is coupled to the C-terminal amino acid of the peptide).

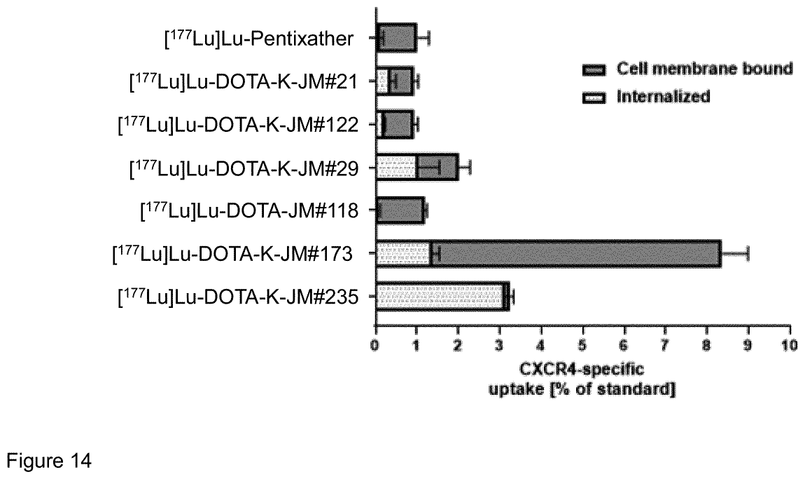

The DOTA-conjugated peptides were radioactively labelled with ¹⁷⁷Lu or with ⁶⁸Ga (see Example further below, FIG. 14-16).

In another particularly preferred embodiment, the complexing agent is the chelator deferoxamine. With other words, the peptide is conjugated to the chelator deferoxamine, wherein the peptide preferably is C-terminally conjugated to deferoxamine. Deferoxamine is also known as desferrioxamine. Deferoxamine-conjugated peptides are suitable for labelling with radioactive nuclides, e.g. ⁶⁸Ga, ¹⁷⁷Lu and ⁸⁹Zr. Consequently, these peptides are useful in applications in diagnostic and therapeutic approaches. The inventors confirmed the suitability of radiolabeled deferoxamine-conjugated peptides as tumor imaging probes and as probes for analyzing the distribution of the peptides in e.g. mouse models. To this end, the inventors synthesized the following deferoxamine-conjugated peptides: C-deferoxamine linked JM #122 (SEQ ID NO.: 51), C-deferoxamine linked JM #194 (SEQ ID NO.: 91), C-deferoxamine linked peptide of SEQ ID NO.: 163 and C-deferoxamine linked peptide of SEQ ID NO.: 164, wherein C indicates the additional cysteine that was coupled to the C-terminal amino acid of the peptides and deferoxamine was conjugated to the peptides via this additional cysteine as (succinimido-propionyl-desferrioxamine) acetate. The deferoxamine-conjugated peptides were radioactively labelled with ⁸⁹Zr.

As an example, the biodistribution of C-deferoxamine linked JM #122 (SEQ ID NO.: 51) radioactively labelled with ⁸⁹Zr was analyzed in mice. To do so, the labeled conjugate was intravenously injected into the tail vein of immunodeficient mice, followed by localization and quantification of radioactivity within the body using positron emission tomography (PET). The analysis revealed a rapid (5 min post injection) absorption of the peptide by the kidneys and subsequent release into the bladder.

The peptide JM #122 (SEQ ID NO.: 51) was derived from JM #21 (SEQ ID NO.: 23) by replacing the cysteine at position 10 by a serine. The peptide of SEQ ID NO.: 163 was derived from JM #143 (SEQ ID NO.: 54) by replacing the cysteine at position 10 by a serine. The peptide of SEQ ID NO.: 164 was derived from JM #198 (SEQ ID NO.: 95) by replacing the cysteine at position 10 by a serine. The replacement of the cysteine by serine facilitated the coupling of deferoxamine to the additional cysteine that was coupled to the C-terminal amino acid of the peptides. The peptide JM #194 (SEQ ID NO.: 91) has no cysteine so that no amino acid replacement was performed before deferoxamine-conjugation to this peptide.

A third aspect of the invention is directed to a peptide consisting of one of the following amino acid sequences, selected from any of the groups 1 to 11, wherein
Group 7 consists of

(SEQ ID NO.: 70, JM#173)
d-ILRWSRK-NH2
(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 71, JM#174)
d-ILRWSRKLP-NH2
(SEQ ID NO.: 95, JM#198)
ILRWSRK(Glu-Ste)LPCVS
(SEQ ID NO.: 104, JM#213)
ILRWSRK(Glu-Lau)LPCVS
(SEQ ID NO.: 108, JM#217)
ILRWSRK(Glu-Ole)LPCVS
(SEQ ID NO.: 117, JM#226)
ILRWSRK(C16diacid)LPCVS
(SEQ ID NO.: 118, JM#227)
ILRWSRK(Glu-C16diacid)LPCVS
(SEQ ID NO.: 119, JM#228)
ILRWSRK(C18diacid)LPCVS
(SEQ ID NO.: 120, JM#229)
ILRWSRK(Glu-C18diacid)LPCVS
(SEQ ID NO.: 121, JM#230)
ILRWSRK(OEG-OEG-γGlu-C18diacid)LPCVS
(SEQ ID NO.: 130, JM#235)
d-LLRWSRK(Glu-Pal)-NH2
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 133, JM#238)
d-ILRWSRK(Glu-Pal)-NH2
(SEQ ID NO.: 139, JM#244)
ILRWSRK(C16diacid)-NH2
(SEQ ID NO.: 140, JM#245)
ILRWSRK(C18diacid)-NH2
(SEQ ID NO.: 141, JM#246)
ILRWSRK(Glu-C16diacid)-NH2
(SEQ ID NO.: 142, JM#247)
ILRWSRK(Glu-C18diacid)-NH2
(SEQ ID NO.: 143, JM#248)
d-LLRWSRK(C16diacid)-NH2
(SEQ ID NO.: 144, JM#249)
d-LLRWSRK(C18diacid)-NH2
(SEQ ID NO.: 145, JM#250)
d-LLRWSRK(Glu-C16diacid)-NH2
(SEQ ID NO.: 146, JM#251)
d-LLRWSRK(Glu-C18diacid)-NH2
(SEQ ID NO.: 147, JM#252)
ILRWSRK(Ara)LPCVS
(SEQ ID NO.: 148, JM#253)
ILRWSRK(Glu-Ara)LPCVS
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 151, JM#256)
d-LLRWSRK(Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2
(SEQ ID NO.: 159, JM#262)
ILRWSRK(Myr)-NH2
(SEQ ID NO.: 161, JM#264)
LVRYTKK(Glu-Pal)-NH2
(SEQ ID NO.: 162, JM#265)
d-LVRYTKK(Glu-Pal)-NH2
(SEQ ID NO.: 163)
ILRWSRK(Pal-Glu)LPSVS

Group 1 consists of

(SEQ ID NO.: 20, JM#18)
ILRWSRKMPCLS
(SEQ ID NO.: 55, JM#144)
ILRWSRK(Pal)LPCVS
(SEQ ID NO.: 89, JM#192)
ILRWSRK(Glu-Pal)-NH2
(SEQ ID NO.: 91, JM#194)
d-LMRWSRK(Glu-Pal)-NH2
(SEQ ID NO.: 150, JM#255)
ILRWSRK(Glu-Ste)-NH2

Group 2 consists of

(SEQ ID NO.: 69, JM#172)
ILRWSRK(Pal)L-NH2
(SEQ ID NO.: 75, JM#178)
d-LMRWSRK(Pal)MP-NH2
(SEQ ID NO.: 77, JM#180)
d-LMRWSRK(Pal)-NH2
(SEQ ID NO.: 80, JM#183)
ILRWSRK(Ole)LPCVS
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 152, JM#257)
d-LLRWSRK(Glu-Ste)-NH2

Group 3 consists of

(SEQ ID NO.: 53, JM#141)
d-LLRWSRK(Pal)MPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 66, JM#169)
IVRWSKK(Pal)VPCVS
(SEQ ID NO.: 67, JM#170)
ILRWSRK(Pal)-NH2
(SEQ ID NO.: 68, JM#171)
ILRWSRK(Pal)LP-NH2
(SEQ ID NO.: 88, JM#191)
ILRWSRKK(Glu-Pal)-NH2
(SEQ ID NO.: 90, JM#193)
ILRWSRKLK(Glu-Pal)-NH2
(SEQ ID NO.: 96, JM#199)
ILRWSRKK(Glu-Ste)LPCVS
(SEQ ID NO.: 98, JM#203)
ILRWSRK(Glu-Dec)LPCVS
(SEQ ID NO.: 100, JM#205)
ILRWSRK(Glu-Ste)LPCVS
(SEQ ID NO.: 107, JM#216)
ILRWSRK(Myr)LPCVS
(SEQ ID NO.: 149, JM#254)
ILRWSRK(Ste)-NH2
(SEQ ID NO.: 157, JM#260)
d-LLRWSRK(Glu-Myr)-NH2
(SEQ ID NO.: 160, JM#263)
ILRWSRK(Glu-Myr)-NH2

Group 4 consists of

(SEQ ID NO.: 10, JM#8)
ILRWSRKVPCVS
(SEQ ID NO.: 12, JM#10)
IFRWSRKVPCVS
(SEQ ID NO.: 29, JM#27)
MLRWSRKMPCVS
(SEQ ID NO.: 36, JM#34)
MMRWSRKMPCVS
(SEQ ID NO.: 41, JM#39)
MLRWSRKLPCVS
(SEQ ID NO.: 51, JM#122)
ILRWSRKLPSVS
(SEQ ID NO.: 52, JM#140)
d-LLRWSRK(Glu-Pal)MPCVS
(SEQ ID NO.: 59, JM#149)
ILRWSRK(Pal)MPCLS
(SEQ ID NO.: 92, JM#195)
d-LMRWSRKK(Glu-Pal)-NH2
(SEQ ID NO.: 97, JM#200)
ILRWSRK-AcLPCVS
(SEQ ID NO.: 105, JM#214)
ILRWSRK(Lau)LPCVS
(SEQ ID NO.: 151, JM#256)
d-LLRWSRK(Ste)-NH2
(SEQ ID NO.: 159, JM#262)
ILRWSRK(Myr)-NH2

Group 5 consists of

(SEQ ID NO. 3, JM#1)
ILRWSKKVPCVS
(SEQ ID NO. 4, JM#2)
IFRWSKKVPCVS
(SEQ ID NO. 5, JM#3)
IVRWSRKVPCVS
(SEQ ID NO. 6, JM#4)
IVRWSHKVPCVS
(SEQ ID NO. 7, JM#5)
IVRWSKKLPCVS
(SEQ ID NO. 8, JM#6)
IVRWSKKIPCVS
(SEQ ID NO. 9, JM#7)
IVRWSKKFPCVS
(SEQ ID NO. 11, JM#9)
ILRWSHKVPCVS
(SEQ ID NO. 13, JM#11)
IFRWSHKVPCVS
(SEQ ID NO. 14, JM#12)
IVRWSKKMPCVS
(SEQ ID NO. 16, JM#14)
IVRWSKKVPCd-VS
(SEQ ID NO. 17, JM#15)
ILRWSRKVPCd-VS
(SEQ ID NO. 18, JM#16)
IIRWSRKMPCVS
(SEQ ID NO. 25, JM#23)
ILRWSRKVPSVS
(SEQ ID NO. 26, JM#24)
ILRWSRKMPSVS
(SEQ ID NO. 27, JM#25)
Ac-SLRWSRKMPCVS
(SEQ ID NO. 28, JM#26)
d-Ac-SLRWSRKMPCVS
(SEQ ID NO. 30, JM#28)
d-MLRWSRKMPCVS
(SEQ ID NO. 31, JM#29)
d-LLRWSRKMPCVS
(SEQ ID NO. 32, JM#30)
d-FLRWSRKMPCVS
(SEQ ID NO. 33, JM#31)
GLRWSRKMPCVS
(SEQ ID NO. 34, JM#32)
Ac-SMRWSRKMPCVS
(SEQ ID NO. 35, JM#33)
d-Ac-SMRWSRKMPCVS
(SEQ ID NO. 37, JM#35)
d-MMRWSRKMPCVS
(SEQ ID NO. 38, JM#36)
d-LMRWSRKMPCVS
(SEQ ID NO. 39, JM#37)
d-FMRWSRKMPCVS
(SEQ ID NO. 40, JM#38)
d-GMRWSRKMPCVS
(SEQ ID NO. 42, JM#40)
d-MLRWSRKLPCVS
(SEQ ID NO. 43, JM#41)
Id-LRWSRKLPCVS
(SEQ ID NO. 44, JM#42)
Id-LRWSRKMPCVS
(SEQ ID NO. 45, JM#43)
Md-LRWSRKLPCVS
(SEQ ID NO. 46, JM#44)
Md-LRWSRKMPCVS
(SEQ ID NO. 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS
(SEQ ID NO.: 106, JM#215)
ILRWSRK(Dec)LPCVS
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 158, JM#261)
d-LLRWSRK(Myr)-NH2
(SEQ ID NO.: 163)
ILRWSRK(Pal-GIu)LPSVS
(SEQ ID NO.: 164)
ILRWSRK(Glu-Ste)LPSVS

Group 6 consists of

(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 164)
ILRWSRK(Glu-Ste)LPSVS

Group 8 consists of

(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2

Group 9 consists of

(SEQ ID NO.: 27, JM#25)
Ac-SLRWSRKMPCVS
(SEQ ID NO.: 30, JM#28)
d-MLRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 52, JM#140)
d-LLRWSRK(Glu-Pal)MPCVS
(SEQ ID NO.: 75, JM#178)
d-LMRWSRK(Pal)MP-NH2
(SEQ ID NO.: 106, JM#215)
ILRWSRK(Dec)LPCVS
(SEQ ID NO.: 158, JM#261)
d-LLRWSRK(Myr)-NH2
(SEQ ID NO.: 160, JM#263)
ILRWSRK(Glu-Myr)-NH2

Group 10 consists of

(SEQ ID NO.: 55, JM#144)
ILRWSRK(Pal)LPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 69, JM#172)
ILRWSRK(Pal)L-NH2
(SEQ ID NO.: 77, JM#180)
d-LMRWSRK(Pal)-NH2
(SEQ ID NO.: 88, JM#191)
ILRWSRKK(Glu-Pal)-NH2
(SEQ ID NO.: 89, JM#192)
ILRWSRK(Glu-Pal)-NH2
(SEQ ID NO.: 90, JM#193)
ILRWSRKLK(Glu-Pal)-NH2
(SEQ ID NO.: 91, JM#194)
d-LMRWSRK(Glu-Pal)-NH2
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2

Group 11 consists of
ILRWCRKPC-NH2, wherein the cysteine at position 5 is bound to the cysteine at position 9 via a disulfide bond to form a cyclic peptide (SEQ ID NO.: 109, JM #218)
ILRW(d-C)RKPC-NH2, wherein the d-cysteine at position 5 is bound to the cysteine at position 9 via a disulfide bond to form a cyclic peptide (SEQ ID NO.: 111, JM #219)
IPRW(d-C)RKC-NH2, wherein the d-cysteine at position 5 is bound to the cysteine at position 8 via a disulfide bond to form a cyclic peptide (SEQ ID NO.: 113, JM #220)
ILRWSRKLPCVS, wherein the lysine at position 7 is bound to the carboxyl-terminus via a peptide bond to form a cyclic peptide (SEQ ID NO.: 115, JM #221)
ILRWSKKLPCVS, wherein the lysine at position 6 is bound to the carboxyl-terminus via a peptide bond to form a cyclic peptide (SEQ ID NO.: 116, JM #222)
IPRW(d-C)RKP, wherein the d-cysteine at position 5 is bound to the proline at position 8 via a thioester bond to form a cyclic peptide (SEQ ID NO.: 122, JM #231)
ILRW(d-C)RKP, wherein the d-cysteine at position 5 is bound to the proline at position 8 via a thioester bond to form a cyclic peptide (SEQ ID NO.: 124, JM #232)
IPRW(d-S)RKP, wherein the d-serine at position 5 is bound to the proline at position 8 via an ester bond to form a cyclic peptide (SEQ ID NO.: 126, JM #233)
ILRW(d-S)RKP, wherein the d-serine at position 5 is bound to the proline at position 8 via an ester bond to form a cyclic peptide (SEQ ID NO.: 128, JM #234)
IMRWCRKPC-NH2, wherein the cysteine at position 5 is bound to the cysteine at position 9 via a disulfide bond to form a cyclic peptide (SEQ ID NO.: 153, JM #258)
IPRW(d-C)RKCP-NH2, wherein the d-cysteine at position 5 is bound to the cysteine at position 8 via a disulfide bond to form a cyclic peptide (SEQ ID NO.: 155, JM #259)
wherein d- in front of an amino acid indicates a D-amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol on the preceding amino acid, Ac indicates a substitution of an amino group by an acetyl group, Lau indicates lauric acid on the preceding amino acid, Glu-Lau indicates a lauric acid on the preceding amino acid with a glutamate linker, Glu-Ole indicates an oleic acid on the preceding amino acid with a glutamate linker, C16diacid indicates a saturated C16 fatty diacid on the preceding amino acid, Glu-C16diacid indicates a saturated C16 fatty diacid on the preceding amino acid with a glutamate linker, C18diacid indicates a saturated C18 fatty diacid on the preceding amino acid, Glu-C18diacid indicates a saturated C18 fatty diacid on the preceding amino acid with a glutamate linker, OEG-OEG-γGlu-C18diacid indicates a saturated C18 fatty diacid on the preceding amino acid with an OEG-OEG-gamma glutamate linker, OEG represents the residue of 8-amino-3,6-dioxaoctanoic acid, Ara indicates a saturated C20 fatty acid on the preceding amino acid, and Glu-Ara indicates a saturated C20 fatty acid on the preceding amino acid with a glutamate linker,
and wherein the peptide is coupled to a polymer.

In other words, the third aspect of the invention relates to a conjugate in which the peptide according to the invention is coupled to a polymer.

The polymer preferably is coupled to the peptide via a cysteine residue. In case the peptide has no cysteine, the polymer is coupled to the peptide via an additional cysteine that is coupled to the C-terminal amino acid of the peptide.

The polymer preferably is C-terminally coupled to the peptide. In case the peptide has an amino acid other than cysteine as C-terminal amino acid, the polymer is coupled to the peptide via an additional cysteine that is coupled to the C-terminal amino acid of the peptide. In case the peptide has cysteine as C-terminal amino acid, the polymer is coupled to the C-terminal cysteine of the peptide or the polymer is coupled to the peptide via an additional cysteine that is coupled to the C-terminal cysteine of the peptide.

The polymer may alternatively be coupled to the peptide via a lysine residue. The above statements regarding the coupling of the polymer via a cysteine residue likewise apply to the coupling of the polymer via a lysine residue.

Polymer-coupled peptides have been shown to have a higher relative stability in human plasma as well as a longer biological availability. Polymers change the physical and chemical properties of the coupled peptide, e.g. hydrophilic properties and, consequently, its size, which inhibits the renal excretion of the peptide. Furthermore, the coupled polymers envelope the peptides, protecting them advantageously from degradation by protease and antibody activity. The peptide activity is enhanced by the coupled polymers.

In a preferred embodiment, the polymer-coupled peptide is coupled to a further peptide. The dimerization of the polymer-coupled peptides is further enhancing their activity.

A preferred polymer is polyethylene glycol (PEG). With other words, the peptide is preferably coupled to PEG.

An also preferred polymer is a poly(vinyl alcohol) (PVA). With other words, the peptide is preferably coupled to a poly(vinyl alcohol). PVA provides an alternative to PEG in case a patient has developed anti-PEG antibodies.

An also preferred polymer is a poly(vinyl pyrrolidone) (PVP). With other words, the peptide is preferably coupled to a poly(vinyl pyrrolidone). PVA provides a further alternative to PEG in case a patient has developed anti-PEG antibodies.

The coupled polymers have a suitable molecular weight, for example between 5 and 20 kDa. Other molecular weights are possible if necessary, depending on the application. In inhibiting HIV-1 infection, the 20 kDa variant was more active than the 5 kDa variant, while in antibody competition they showed similar activity.

In a preferred embodiment, the coupled polymers may be coupled to two copies of identical monomeric peptides. Coupling of two different monomeric peptides is also possible. It is preferred that the peptide copies are coupled on one end of the polymer. It is also possible that the peptide copies are coupled to different ends of the polymer (telechelic peptide conjugates). It has been shown that polymers with peptide copies on one end (SC066) have a higher activity than polymers with peptide copies coupled to different ends of the polymer (SC029).

With other words, it is preferred that the polymer of the polymer-coupled peptide is coupled to a further peptide, wherein the further peptide preferably is a copy of the peptide of the invention. The two peptides are preferably coupled on one end of the polymer.

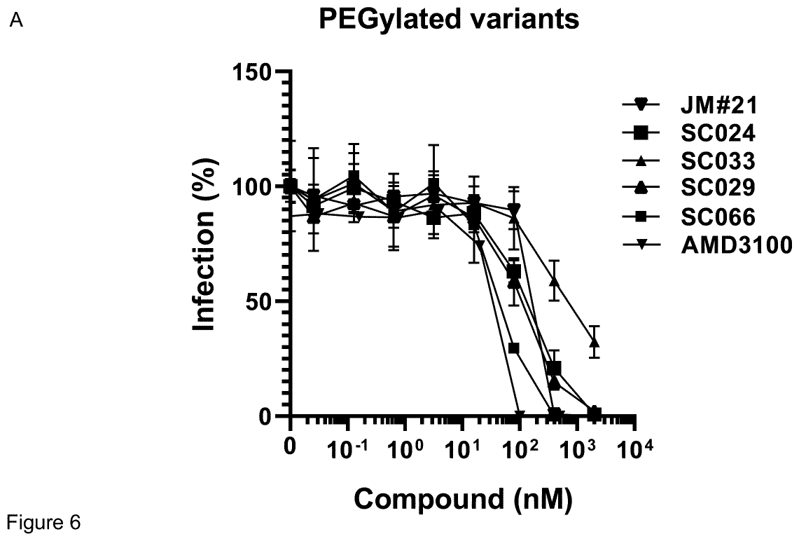

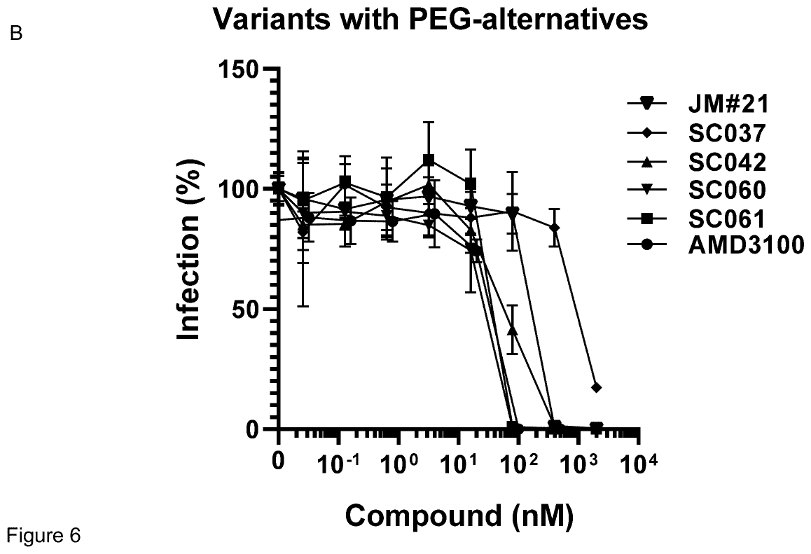

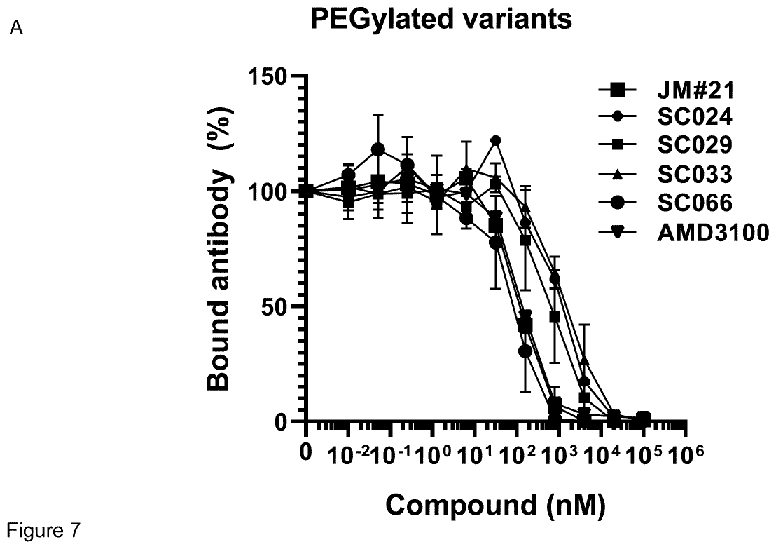

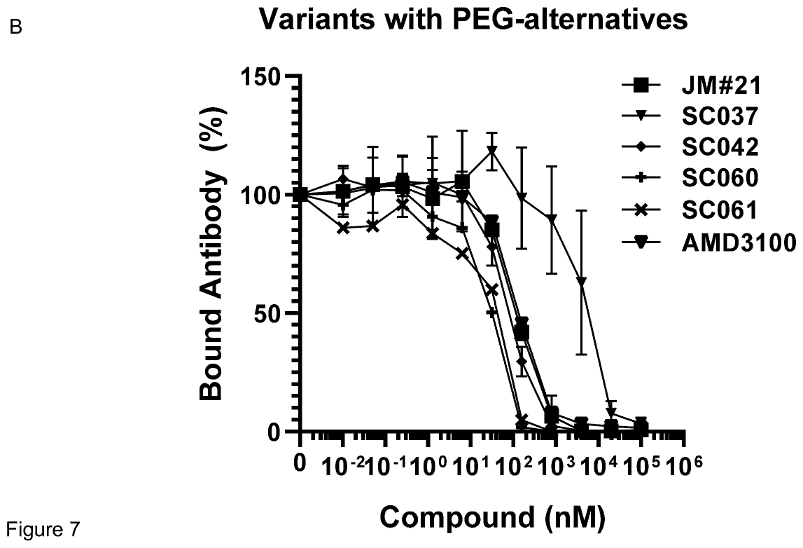

The effect of the polymer-coupled variants is illustrated in FIG. 6 and FIG. 7. The polymers were coupled to peptide JM #21 (SEQ ID NO.: 23). The PEG-coupled peptides SC024 (average molecular mass 20 kDa), SC033 (average molecular mass 5 kDa), SC029 (average molecular mass 20 kDa), and SC066 (average molecular mass 20 kDa) show an activity estimated by blocking HIV-1 infection which is comparable to the state of the art CXCR4-antagonist AMD3100 and JM #21 (SEQ ID NO.: 23) (FIG. 6A). The same is true for the antibody competition assay, wherein the PEG-coupled derivatives bound with a similar affinity to CXCR4 as JM #21 (SEQ ID NO.: 23) and AMD3100 (FIG. 7A). It has been shown that polymers with two peptide copies on one end (SC066) have a higher activity than polymers with peptide copies coupled to different ends of the polymer (SC029). In the antibody competition assay, the derivative SC066 (two peptide copies on one end of PEG) showed an even higher activity than JM #21 (SEQ ID NO.: 23) and AMD3100. For each peptide and assay, three independent experiments (FIG. 6) or two independent experiments (FIG. 7) were carried out. The error bars refer to the standard deviation.

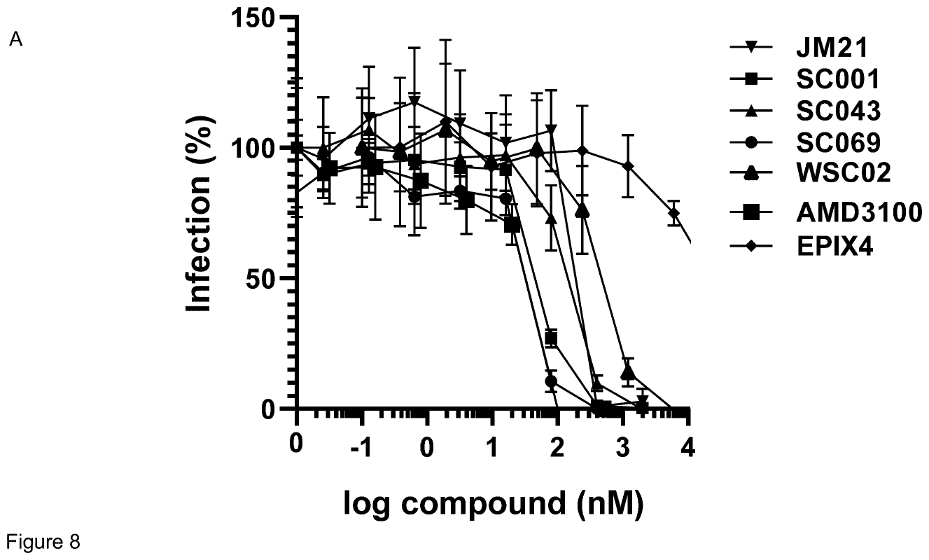

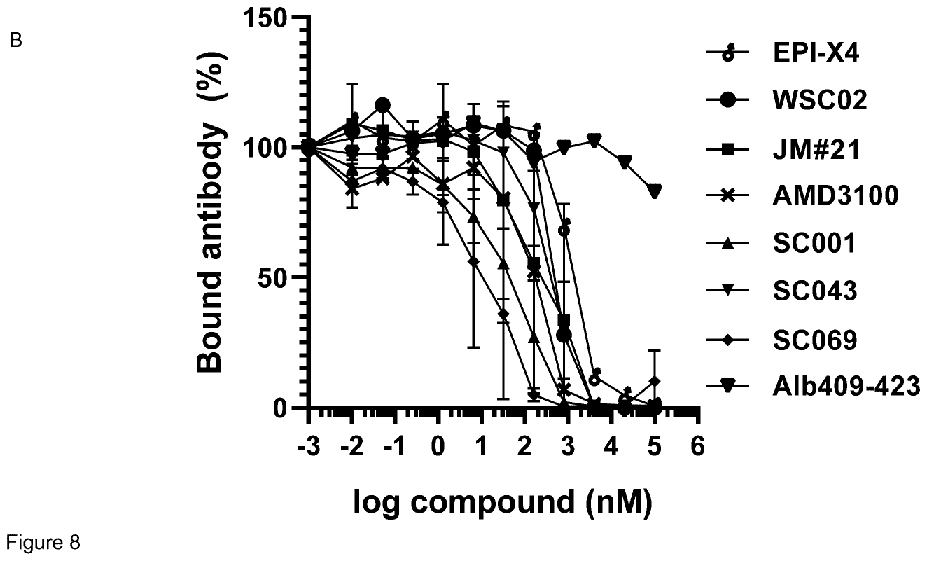

The activities of the PVP-coupled peptides SC037 (average molecular mass 20 kDa) and SC060 (two peptides on one end, average molecular mass 20 kDa) as well as of the PVA-coupled peptides SC042 (average molecular mass 20 kDa) and SC061 (two peptides on one end, average molecular mass 20 kDa) are shown in FIGS. 8B and 9B. All derivatives had a comparable activity as JM #21 (SEQ ID NO.: 23) and AMD3100, wherein the derivatives SC060 and SC061, harboring two peptide copies on one end of PVP and PVA, respectively, showed an even higher activity. For each peptide, three independent experiments were carried out. The error bars refer to the standard deviation.

In a further preferred embodiment, the PEG-coupled peptide is coupled to 1,2-distearoyl-sn-glycero-3-phosphoethanolamine (DSPE). DSPE is a phospholipid which has been shown to increase the relative stability of the peptides in human plasma as well as the biological availability. DSPE is coupled to the peptide via PEG, so it has the formula 1,2-distearoyl-sn-glycero-3-phosphoethanolamine-N-[amino(polyethylene glycol)-2000]. It is also preferred that the DSPE is coupled to a further peptide (via PEG). The further peptide preferably is a copy of the peptide of the invention. The two peptides are preferably coupled on one end of PEG.

In FIG. 8, it can be seen that DSPE-coupled derivative SC001 (one peptide copy) and SC069 (two peptide copies on one end) have a higher activity than cholesterol-coupled SC043, JM #21 (SEQ ID NO.: 23), EPI-X4 (SEQ ID NO.: 1), WSC02 (SEQ ID NO.: 2), AMD3100 and Albumin fragment Alb409-423, as shown by an HIV-inhibition assay (FIG. 8A) and an antibody competition assay (FIG. 8B). The derivative with two peptide copies on one end (SC069) was shown to have a higher activity than the derivative with one peptide copy (SC001).

Regarding the relative stability of the modified peptides in human plasma, the inventors found that the cholesterol-coupled SC043 has the highest stability (relative activity of 100% as measured after 8 hours of plasma incubation), followed by the PEG-coupled peptide SC033 (average molecular mass 5 kDa) (relative activity of 87% as measured after 2 hours of plasma incubation), which is followed by the PEG-coupled peptides SC029 (average molecular mass 20 kDa) and SC024 (average molecular mass 20 kDa).

In SC043, cholesterol is coupled via PEG (average molecular mass 5 kDa) to the cysteine at position 10 of the peptide JM #21 (SEQ ID NO.: 23). Accordingly, in a preferred embodiment, the PEG-coupled peptide of the invention is coupled to cholesterol, wherein cholesterol is coupled to the peptide via PEG.

The polymers were also coupled to the peptides JM #29 (SEQ ID NO.: 31), JM #118 (SEQ ID NO.: 50) and JM #173 (SEQ ID NO.: 70). In case of JM #29 (SEQ ID NO.: 31) the polymer was coupled to the cysteine at position 10 of the peptide. In case of JM #118 (SEQ ID NO.: 50) and JM #173 (SEQ ID NO.: 70), an additional cysteine was coupled to the C-terminal amino acid of the peptides and the polymer was coupled to the peptides via this additional cysteine. The synthesis of the polymer-coupled peptides was performed in the same manner as the synthesis of the polymer-coupled derivatives of the peptide JM #21 (SEQ ID NO.: 23). The inventors expect that the polymer-coupled derivatives of the peptides JM #29 (SEQ ID NO.: 31), JM #118 (SEQ ID NO.: 50) and JM #173 (SEQ ID NO.: 70) show an activity that is similar to the activity seen with the polymer-coupled derivatives of the peptide JM #21 (SEQ ID NO.: 23) (FIG. 6-8). The polymer-coupled derivatives of the peptides JM #29 (SEQ ID NO.: 31), JM #118 (SEQ ID NO.: 50) and JM #173 (SEQ ID NO.: 70) are highly stable in human plasma.

The DSPE-PEG-coupled peptides can be used for the formulation of a nanocarrier for a drug or a permeation enhancer. In this case, the peptide portion of the modified peptide shows to the outside of the nanocarrier while the DSPE portion of the modified peptide shows to the inside of the nanocarrier (micelle formation). The drug may be, for example, an anti-cancer drug such as a chemotherapeutic agent such as doxorubicin. The nanocarrier is suitable for improving the targeting of the drug to its target site.

The permeation enhancer preferably is an intestinal permeation enhancer that facilitates oral delivery of macromolecules such as the DSPE-PEG-coupled peptides of the invention. The permeation enhancer may be, for example, sodium N-[8-(2-hydroxybenzoyl)amino]caprylate (SNAC).

In a preferred embodiment, the polymer-coupled peptide is coupled to a chelator. The chelator preferably is conjugated to the peptide via a lysine residue. The statements made above regarding the coupling of a polymer to the peptide via a lysine residue likewise apply to the coupling of a chelator to the peptide. The resulting conjugate is preferably labeled with a radioactive nuclide via the chelator. This also applies to polymer-coupled peptides in which the polymer of the polymer-coupled peptide is coupled to a further peptide as described above. In other words, the polymer-coupled peptide may be coupled to a further peptide via the polymer and further to a chelator, wherein the further peptide preferably is a copy of the peptide of the invention. The polymer preferably is PEG.

The chelator may also be coupled to the polymer-coupled peptide via the polymer, for example in a manner that corresponds to the coupling of a further peptide to the polymer-coupled peptide.

Suitable chelators are, for example, dodecane tetraacetic acid (DOTA), deferoxamine. 1,4,7-Triazacyclononane-1,4,7-triacetic acid (NOTA), N,N′-bis-[2-hydroxy-5-(carboxyethyl)benzyl]ethylenediamine-N,N′-diacetic acid (HBED-CC), Triazacyclononane-phosphinic acid (TRAP) or Tris(hydroxypyridinone) (THP).

A fourth aspect of the invention is related to a peptide consisting of two identical monomeric peptides according to the invention, wherein the monomeric peptides are linked to each other via a cysteine bridge which is formed between the monomeric peptides to form a dimeric peptide. Dimeric peptides consisting of two different monomeric peptides are also possible, though dimeric peptides consisting of two identical monomeric peptides are more effective. The dimeric peptides show a higher activity compared to a double amount of the respective monomeric peptides (both versions are disclosed already in EP3007717).

In a preferred embodiment, the dimeric peptide is coupled to a complexing agent such as, for example, the chelator DOTA. The complexing agent preferably is conjugated to the dimeric peptide via a lysine residue. The statements made above regarding the coupling of a polymer to the peptide via a lysine residue likewise apply to the coupling of a chelator to the dimeric peptide. The statements made above regarding peptides that are conjugated to a complexing agent likewise apply to dimeric peptides coupled to a complexing agent.

In a preferred embodiment, the dimeric peptide is coupled to a polymer. The statements made above regarding polymer-coupled peptides likewise apply to polymer-coupled dimeric peptides.

A fifth aspect of the invention is related to a pharmaceutical composition comprising the inventive peptide together with at least one pharmaceutically acceptable carrier, mesoporous nanoparticles, cryoprotectant, lyoprotectant, excipient and/or diluent. The pharmaceutical composition may further comprise binders, disintegrates, glidants, lubricants, coloring agents, sweetening agents, flavoring agents, preservatives, and/or the like. Ingredients are selected for their use in specific applications. Mesoporous nanoparticles, for example, are advantageous for a sustained release of the peptides. Packaging the peptides in mesoporous nanoparticles such as mesoporous silica nanoparticles increases the bioavailability of the peptides in vivo.

The peptides may be packaged in a lipid delivery system such as a self-emulsifying drug delivery system (SEDDS). The peptides have optimal properties for the lipid delivery system, since the peptides are very small and highly positively charged or already lipophilic what helps with packaging.

The peptides may be formulated together with a permeation enhancer. The permeation enhancer preferably is an intestinal permeation enhancer that facilitates oral delivery of the peptides of the invention. The permeation enhancer may be, for example, sodium N-[8-(2-hydroxybenzoyl)amino]caprylate (SNAC).

The fatty acid-conjugated peptide derivatives of the invention may be formulated together with free fatty acids in order to provide a nanocarrier (micelle formation). The nanocarrier can then be loaded with, for example, a drug or a permeation enhancer. The drug may be, for example, an anti-cancer drug such as a chemotherapeutic agent such as doxorubicin. The nanocarrier is suitable for improving the targeting of the drug to its target site. The permeation enhancer may be, for example, SNAC.

A sixth aspect of the invention is related to the inventive peptide or the inventive pharmaceutical composition for use in medicine.

A seventh aspect of the invention is related to the use of the inventive peptide or the inventive pharmaceutical composition for the preparation of a formulation for oral administration, inhalation, intravenous administration, topical administration, intranasal administration, intraperitoneal administration, subcutaneous administration and/or any other injectable form. The pharmaceutical composition may be administered, for example, in the form of liquid formulations including solutions, suspensions and emulsions, and in the form of pills, tablets, film tablets, coated tablets, capsules, liposomal formulations, micro- and nano-formulations, and powders.

In a preferred embodiment, the pharmaceutical composition is prepared as a lyophilized formulation of a buffered liquid formulation.

In a preferred embodiment, the pharmaceutical composition is prepared as a formulation for oral administration. In this case, the peptides may be formulated together with a permeation enhancer as described above.

An eighth aspect of the invention is related to the inventive peptide or the inventive pharmaceutical composition for use in the treatment of disorders of hematopoiesis, in particular for support of the mobilization, proliferation and migration of stem cells; in the treatment of wounds, in particular wounds caused by burning; in the treatment of viral diseases, in particular infections with HIV-1, HIV-2, SARS-CoV-2, Cytomegalovirus, Herpes simplex virus (type 1 and 2), Varicella zoster virus, Hepatitis A and Hepatitis B virus, Influenza virus, Polio virus, Rhino virus, Rubella virus, Measles virus, Rabies virus, Rous sarcoma virus, Epstein-Barr Virus; in the treatment of infections caused by bacteria and fungi, in particular Pseudomonas, Candida, S. aureus, in the treatment of infectious processes, abnormal infectious processes; in the treatment of inflammation, in particular of periodontal disease; in the treatment of growth disorders; in the treatment of neuronal diseases, disorders of the blood clotting cascade and hematopoiesis, vascular diseases, diseases of the immune system, for improving wound and bone healing, for use in the treatment of neurological diseases, in particular stroke, Parkinson's disease, Alzheimer's disease, multiple sclerosis; in the treatment of warts, Hypogammaglobulinemia, Immunodeficiency, and Myelokathexis syndrome (WHIM-syndrome) and rheumatoid arthritis; in the treatment of cancers, in particular cancers showing the CXCR4 receptor, preferably cancer of the liver, pancreas, prostate, breast cancer or other solid tumors; in the treatment of lack of mobilization, proliferation and migration of stem cells, T-cell activation as well as support of immunoblasts, preferably of cytotoxic T lymphocytes with programmed cell death receptor 1 (CTL/PD-1); in the treatment of antifibrosis; in the treatment or prevention of scars; in the treatment of cardiologic disorders, in particular heart insufficiency; in the treatment of metabolic disorders, in particular diabetes, and in the treatment of lung diseases, in particular lung fibrosis, bronchitis, chronic obstructive pulmonary disease (COPD).

Experimental data support the efficiency of the claimed peptide derivatives against the diseases mentioned above. At the example of JM #21 (SEQ ID NO.: 23) it could be shown that the peptides counteract growth and migration in vitro and in vivo of Acute Myeloid Leukemia (AML) cells as well as primary patient material and Waldenström's Macroglobulinemia (WM) cells harboring different WHIM-like CXCR4 mutations. This was accompanied by intrinsic changes suppressing oncogenic MAP kinase signaling of AML and WM cells. In relation to AML cells, JM #21 (SEQ ID NO.: 23) efficiently blocked the CXCR4 12G5 epitope of AML cells in a dose dependent manner, inhibited migration of AML cells along a CXCL12 gradient, reduced CXCL12-induced ERK phosphorylation of AML cells, and reduced engraftment potential of primary CXCR4 AML patient samples in NSG mice whereas it has no inhibiting effect on the engraftment potential of CD34+ normal cells. In relation to WM cells, JM #21 (SEQ ID NO.: 23) blocked the CXCR4 12G5 epitope of WM cells in presence or absence of different CXCR4 mutations in a dose dependent manner, impaired migration of WM cells with or without S338X mutation along a CXCL12 gradient, and reduced CXCL12-induced ERK phosphorylation of CXCR4 mutant WM cells in a dose-dependent manner.

A ninth aspect of the invention is related to the inventive peptide or the inventive pharmaceutical composition for use in the prophylaxis and/or treatment of cancer, viral diseases, metabolic disorders, neurologic disorders, diseases of the immune system, or disorders of the blood clotting cascade and hematopoiesis in a mammal, wherein the mammal preferably is a human. The terms “prophylaxis” and “treatment” comprise the fact that a pharmaceutically effective amount of the inventive peptide or the inventive pharmaceutical composition, or salts or hydrates thereof effective to treat the above mentioned conditions, is to be administered to the mammal.

The inventive peptide or the inventive pharmaceutical composition preferably is for use in the prophylaxis and/or treatment of CXCR4-expressing cancer. The CXCR4-expressing cancer preferably is a CXCR4-expressing liver, pancreas, prostate, or breast cancer or another CXCR4-expressing solid tumor. Preferred CXCR4-expressing cancers are also CXCR4-expressing cancers of the hematopoietic system such as AML, WM and B cell lymphoma.

The inventive peptide or the inventive pharmaceutical composition preferably is for use in the treatment of inflammation. This includes the treatment of inflammatory diseases such as atopic dermatitis, allergic asthma, colitis and arthritis.

The inventive peptide or the inventive pharmaceutical composition preferably is for use in the treatment of infections with HIV-1 or HIV-2.

The inventive peptide or the inventive pharmaceutical composition preferably is for use in the treatment of infections with SARS-CoV-2. In infections with SARS-CoV-2, CXCR4-positive cells are implicated in severe disease progression in the lungs.

A tenth aspect of the invention is related to a method for manufacturing the inventive peptide by solid phase synthesis. If this is not possible, e.g. for peptides coupled to polymers, other methods are selected for manufacture of those derivates. In a preferred embodiment, monomeric peptides are provided and coupled under oxidative reaction conditions which are capable to oxidize SH bonds to yield —S—S-bonds.

The peptide of the invention may be coupled to cholesterol. Accordingly, a further aspect of the invention is related to a conjugate in which the peptide according to the invention is coupled to cholesterol. Cholesterol has been shown to increase the relative stability of the peptides in human plasma as well as the biological availability. Cholesterol preferably is conjugated to the peptide via a lysine residue or via a cysteine residue. The statements made above regarding the coupling of a polymer to the peptide via a cysteine residue or via a lysine residue likewise apply to the coupling of cholesterol to the peptide.

In case cholesterol is coupled to the peptide via the cysteine residue, cholesterol is coupled to the peptide via a linker. The linker is chosen to have a suitable length. It is preferred that the linker is PEG. PEG is chosen to have a suitable molecular weight, for example between 5 and 20 kDa. As an example, the inventors have coupled cholesterol via PEG to the cysteine residue of the peptides JM #21 (SEQ ID NO.: 23) and JM #29 (SEQ ID NO.: 31). The inventors have also coupled cholesterol via PEG to an additional cysteine that is coupled to the C-terminal amino acid of the peptides JM #118 (SEQ ID NO.: 50) and JM #173 (SEQ ID NO.: 70).

In case cholesterol is coupled to the peptide via the lysine residue, cholesterol is directly coupled to the peptide or cholesterol is coupled to the peptide via a linker. The linker is chosen to have a suitable length. It is preferred that the linker is a glutamate linker. As an example, the inventors have synthesized and analyzed the peptide of SEQ ID NO.: 81 (JM #184) (JM #21 (SEQ ID NO.: 23) with cholesterol directly coupled to the lysine at position 7 of the peptide).

The peptide of the invention may be coupled to a saturated and/or an unsaturated fatty acid. The saturated and/or unsaturated fatty acid preferably is conjugated to the peptide via a lysine residue. The statements made above regarding the coupling of a polymer to the peptide via a lysine residue likewise apply to the coupling of a saturated and/or an unsaturated fatty acid to the peptide. The saturated and/or unsaturated fatty acid is directly coupled to the peptide or is coupled to the peptide via a linker. The linker is chosen to have a suitable length. It is preferred that the linker is a glutamate linker.

The peptide of the invention may be coupled to a drug. Accordingly, a further aspect of the invention is related to a conjugate in which the peptide according to the invention is coupled to a drug. This conjugate has two active agents, namely the peptide and the drug. The drug preferably is coupled to the peptide via a lysine residue or via a cysteine residue. The statements made above regarding the coupling of a polymer to the peptide via a cysteine residue or via a lysine residue likewise apply to the coupling of the drug to the peptide. The drug may be, for example, an anti-cancer drug such as a chemotherapeutic agent. The peptide is suitable for improving the targeting of the anti-cancer drug to the cancer. The drug may be, for example, an antibody such as an HIV-1 antibody or a receptor-targeting antibody.

As mentioned above, the peptides of the invention are suitable to be coupled to proteins. Proteins to be coupled to the peptides are, for example, antibodies or human serum albumin (HSA). Accordingly, a further aspect of the invention is related to a conjugate in which the peptide according to the invention is coupled to a protein.

In a preferred embodiment, the peptide of the invention is conjugated to human serum albumin (albumin). The peptide is preferably conjugated to albumin via disulfide rebridging method. To this end, the peptide, for example JM #21 (SEQ ID NO.: 23), is preferably conjugated to albumin via an allyl linker. The allyl linker can be connected to disulfide bridges within albumin without destroying the integrity of the protein. It is preferred to protect the cysteine at position 34 within albumin (Cys34), which is the only cysteine within albumin, before the reaction in order to preserve its accessibility for Cys34-conjugating drugs such as aldoxorubicin. Besides its long circulation half-life, albumin accumulates inside solid tumor tissues and inflammatory sites, which are also target sites of the peptides of the invention. Accordingly, the albumin-conjugated peptides are highly stable in human plasma and provide a platform for the targeting of tumors or inflammatory sites. The therapeutic effect of the albumin-conjugated peptides will be achieved via CXCR4. An additional therapeutic effect can be achieved via a drug that is additionally coupled to the albumin (e.g. via Cys34).

The peptide of the invention may also be conjugated to a scaffold protein other than human serum albumin. The scaffold protein may be, for example, avidin.

In a preferred embodiment, the peptide of the invention is conjugated to an antibody. The antibody preferably is a monoclonal antibody that has a plasma circulation half-life comparable to that of albumin. By using a branched linker, heterodimers with peptides targeting other therapeutically important receptors (e.g. somastatin receptor, CCR2, CXCR7) can be fused to the antibody, thereby creating a bispecific antibody construct that targets CXCR4 and another interaction partner at the same time.

In a particularly preferred embodiment, the peptide of the invention is conjugated to a broadly neutralizing HIV-1 antibody (bNAb), thereby creating a bispecific EPI-X4-bNAb construct that is suitable for HIV-1 therapy and prevention. Broadly neutralizing HIV-1 antibodies neutralize multiple HIV-1 viral strains.

In a preferred embodiment, the peptide of the invention is conjugated to a maleimide linker. The maleimide linker preferably is conjugated to the peptide via a cysteine residue. The statements made above regarding the coupling of a polymer to the peptide via a cysteine residue likewise apply to the coupling of a maleimide linker to the peptide. Examples of maleimide linkers are mal-dPEG(3)-mal and mal-PEG-mal (see below). Maleimide linkers are able to interact with Cys34 on human serum albumin. The peptide that is conjugated to the maleimide linker, for example JM #173 (SEQ ID NO.: 70), is supposed to react with albumin in vivo (binding of the peptide to Cys34 on albumin via the maleimide linker) and therefore is highly stable in human plasma, but not lipophilic (as with the fatty acid linked peptide versions). As an example, the inventors have used peptides JM #21 (SEQ ID NO.: 23) and JM #29 (SEQ ID NO.: 31) and added bis-1,13-(3-maleimidopropionyl)amido)-4,7,10-trioxatridecane (mal-dPEG(3)-mal) or alpha,omega-Bis-maleimido poly(ethylene glycol) (PEG-MW 2.000 Da) (mal-PEG-mal) via the peptide cysteine. The inventors further used the peptide JM #173 (SEQ ID NO.: 70) to design the conjugate JM #173-C-mal-PEG-mal, in which the maleimide linker mal-PEG-mal is coupled to an additional cysteine that is coupled to the C-terminal amino acid of JM #173 (SEQ ID NO.: 70).

In a preferred embodiment, the peptide of the invention is conjugated to human serum albumin via the maleimide linker. In this case, the peptide of the invention is conjugated to Cys34 on albumin via the maleimide linker and the conjugation was performed in vitro.

Everything described in relation to the peptides of the invention further above, in particular the preferred embodiments, uses, medical uses and methods, also apply to the peptides coupled to cholesterol, an unsaturated fatty acid, a drug, a protein or a maleimide linker.

The inventors have further synthesized the following peptides:

(SEQ ID NO.: 15, JM#13)
ILRWSRKMPCVS
(SEQ ID NO.: 19, JM#17)
IMRWSRKMPCVS
(SEQ ID NO.: 22, JM#20)
ILRWSRKMPCMS
(SEQ ID NO.: 23, JM#21)
ILRWSRKLPCVS
(SEQ ID NO.: 24, JM#22)
ILRWSRKFPCVS
(SEQ ID NO.: 21, JM#19)
ILRWSRKMPCFS

The peptides of SEQ ID NO.s: 15, 19, 22, 23 and 24 were found to have an inhibitory activity characterized by an IC₅₀ of below 5 nM, as measured in the X4-HIV-1 inhibition assay.

The peptide of SEQ ID NO.: 21 was found to have an inhibitory activity characterized by an IC₅₀ between 5 and 10 nM, as measured in the HIV inhibition assay.

Everything described in relation to the peptides of the invention, In particular the preferred embodiments, uses, medical uses and methods, also apply to the peptides of SEQ ID NO.s: 15, 19, 22, 23, 24 and 21.

The inventors have further synthesized the following peptides, which are part of the present disclosure:

(SEQ ID NO.: 134, JM#239)
d-ILRWSRKEYEK(Pal)EYE
(SEQ ID NO.: 135, JM#240)
d-ILRWSRK(Pal)EK(Pal)

ILRW(d-C)RK(Pal)PC-NH2, wherein the d-cysteine at position 5 is bound to the cysteine at position 9 via a disulfide bond to form a cyclic peptide (SEQ ID NO.: 136, JM #241)
d-ILRW(d-C)RKPC-NH2, wherein the d-cysteine at position 5 is bound to the cysteine at position 9 via a disulfide bond to form a cyclic peptide (SEQ ID NO.: 137, JM #242)
d-ILRW(d-C)RK(Pal)PC-NH2, wherein the d-cysteine at position 5 is bound to the cysteine at position 9 via a disulfide bond to form a cyclic peptide (SEQ ID NO.: 138, JM #243)

The activity of the peptides of SEQ ID NO.s: 134-138 was found to be insufficient.

Disclosed is a method of treatment of a CXCR4-related medical condition in a mammal, wherein the method comprises administering the inventive peptide or the inventive pharmaceutical composition to the mammal, wherein the mammal preferably is a human. The CXCR4-related medical condition particularly comprises disorders of hematopoiesis, in particular for support of the mobilization, proliferation and migration of stem cells; wounds, in particular wounds caused by burning; viral diseases, in particular infections with HIV-1, HIV-2, SARS-CoV-2, Cytomegalovirus, Herpes simplex virus (type 1 and 2), Varicella zoster virus, Hepatitis A and Hepatitis B virus, Influenza virus, Polio virus, Rhino virus, Rubella virus, Measles virus, Rabies virus, Rous sarcoma virus, Epstein-Barr Virus; infections caused by bacteria and fungi, in particular Pseudomonas, Candida, S. aureus, infectious processes, abnormal infectious processes; inflammation, in particular periodontal disease; growth disorders; neuronal diseases, disorders of the blood clotting cascade and hematopoiesis, vascular diseases, diseases of the immune system, for improving wound and bone healing, neurological diseases, in particular stroke, Parkinson's disease, Alzheimer's disease, multiple sclerosis; warts, Hypogammaglobulinemia, Immunodeficiency, and Myelokathexis syndrome (WHIM-syndrome) and rheumatoid arthritis; cancers, in particular cancers showing the CXCR4 receptor, preferably cancer of the liver, pancreas, prostate, breast cancer or other solid tumors; the lack of mobilization, proliferation and migration of stem cells, T-cell activation as well as support of immunoblasts, preferably of cytotoxic T lymphocytes with programmed cell death receptor 1 (CTL/PD-1); antifibrosis; scars; cardiologic disorders, in particular heart insufficiency; metabolic disorders, in particular diabetes, and in the treatment of lung diseases, in particular lung fibrosis, bronchitis, chronic obstructive pulmonary disease (COPD).

Further disclosed is a method of prophylaxis and/or treatment of cancer, viral diseases, metabolic disorders, neurologic disorders, diseases of the immune system, or disorders of the blood clotting cascade and hematopoiesis in a mammal, wherein the method comprises administering the inventive peptide or the inventive pharmaceutical composition to the mammal, wherein the mammal preferably is a human. The cancer preferably is a CXCR4-expressing cancer. The CXCR4-expressing cancer preferably is a CXCR4-expressing liver, pancreas, prostate, or breast cancer or another CXCR4-expressing solid tumor. Preferred CXCR4-expressing cancers are also CXCR4-expressing cancers of the hematopoietic system such as AML, WM and B cell lymphoma. The diseases of the immune system preferably are inflammatory diseases such as atopic dermatitis, allergic asthma, colitis and arthritis. The viral diseases preferably are infections with HIV-1, HIV-2 or SARS-CoV-2.

### Experimental Procedures

HIV-1 inhibition assay. Viral stocks of CXCR4-tropic NL4-3 were generated by transient transfection of 293T cells with proviral DNA as described (Munch et al., 2007). The next day the transfection mixture was removed and fresh medium containing 2.5% FCS was added. 2 days after transfection the supernatant was harvested and cell debris were removed by centrifugation. Aliquots were stored at −80° C. For infection of TZM-bl cells in presence of inhibitors, cells were seeded at a density of 1×10\hat{ }5 cells/ml in 70 μl DMEM containing 2.5% FCS. Compounds were diluted in PBS and 10 μl were added. After 15 minutes of incubation cells were inoculated with 20 μl of diluted virus. Infection rates were determined three days later using Gal-Screen system (Applied Biosystems).

Antibody competition assay. Competition of compounds with antibody binding was performed on SupT1 cells. For that cells were washed in PBS containing 1% FCS and 50,000 cells were then seeded per well in a 96 V-well plate. Buffer was removed and plates were precooled at 4° C. Compounds were diluted in PBS and antibody (clone 12G5, APC labelled) was diluted in PBS containing 1% FCS. The antibody was used at a concentration closed to its determined Kd. 15 μl compounds were then added to the cells and 15 μl antibody immediately afterwards. Plates were incubated at 4° C. in the dark for 2 hours. Afterwards cells were washed twice with PBS containing 1% FCS and fixed with 2% PFA. Antibody binding was analyzed by flow cytometry (FACS CytoFLEX; Beckman Coulter®).

Stability assay. Whole blood was collected from healthy donors in EDTA tubes and directly used or subsequently centrifuged for 15 min at 2,500×g to obtain plasma. Plasma from 6 donors was pooled and stored in aliquots at −80° C. Compounds were 200-fold diluted in human plasma or whole human blood to reach final concentrations of 20 μM. The t=0 sample was immediately taken and stored at −80° C. Plasma/compound or blood/compound mixture was then transferred to 37° C. and shook at 350 rpm. At given time points samples were taken and stored at −80° C.°. For measuring the functional activity of the plasma/peptide samples, the mixtures were thawed and diluted in ice cold PBS. 12G5-APC antibody competition was then performed as described before. For blood/peptide functional stability, samples were thawed and centrifuged at 14,000 rpm to remove cells and debris. The supernatant was then diluted in PBS and 12G5-antibody competition assay was performed. After the 2 hours incubation, the cells were washed and 50 μl of 1-step-Fix/Lyse solution (Thermo Fisher #00-5333-54) was added for 15 minutes at room temperature. Afterwards cells were washed again and analyzed for bound antibody.

Stability assay in human S9 liver fractions. Pooled human liver S9 fractions were obtained from Thermo Fisher Scientific at a total protein concentration of 20 mg/ml. Fractions were stored in 25 μl aliquots at −80° C. For stability experiments, they were diluted to a final concentration of 0.5 mg/ml in Tris buffer. Cofactors (or buffer) were added immediately before start of the experiment (NADPH: 1 mM, UDPGA: 0.5 mM, GSH: 2.5 mM, PAPS: 0.05 mg/ml, Sigma Aldrich). The reaction was started by adding peptides or compounds at a concentration of 20 μM to the mixture and gentle agitation at 37° C. Determination of enzymatic stability was performed as described for plasma.

In vivo stability assay. 100 μl of a 700 μg/ml stock solution of the peptide in 0.9% NaCl was intravenously injected in the tail vein of C57BL/6NCrl (BL6) mice. 4 hours post injection, mice were killed by cervical dislocation. Mouse plasma was obtained by heart punctation. Blood was 19:1 diluted with 0.16 M NaEDTA and centrifuged at 2000×g for 20 min at 4° C. to obtain plasma. Plasma was stored at −80° C. till remaining peptide activity in the plasma was determined by 12G5 antibody competition assay. Activities were compared to the activity of a peptide spiked plasma sample.

ERK/AKT signaling assay. CXCL12 induced ERK and AKT phosphorylation was determined in SupT1 cells. For this, 100,000 cells were seeded per well in a 96-V well plate in 100 μl medium supplemented with 1% FCS. Cells were incubated for 2 hours at 37° C. before 5 μl of compounds were added. After 15 min incubation at 37° C. cells were stimulated by adding 5 μl CXCL12 diluted in PBS to reach a final concentration of 100 ng/ml. Cells were further incubated for 2 min before the reaction was stopped by adding 20 μl of 10% PFA. Cells were fixed for 15 min at 4° C. before PFA was removed and cells permeabilized by adding 100 μl ice cold methanol. After 15 min at 4° C. the methanol was removed, cells were washed and 30 μl primary antibody was added (phospho-p44/42 MAPK (Erk1) (Tyr204)/(Erk2) (Tyr187) (D1H6G) mouse mAb #5726; phosphor-Akt (Ser473) (193H12) rabbit mAb #4058 Cell Signaling) for 1 hour at 4° C. After the antibody was removed and cells were washed secondary antibody was added for 30 min. Cells were washed afterwards and subsequently analyzed by flow cytometry.

Migration assay. Migration assays were performed using 96-well transwell assay plates (Corning Incorporated, Kennebunk, Me., USA) with 5 μm polycarbonate filters. First, the lower chambers were filled with 235 μl assay buffer (RPMI supplemented with 0.1% BSA) with or without 100 ng/ml CXCL12 and serial dilutions of the CXCR4-inhibiting compounds (in assay buffer). Next, 75 μL (0.5×105 cells) of Jurkat cells (in assay buffer), together with/without the compounds, were added into the upper chambers. After 4 h at 37° C. (5% CO2), 100 μL of the lower chambers were transferred to a fresh 96 V-well plate and analyzed with Cell-Titer-Glo® assay (Promega, Madison, Wis., USA). The percentage of migrated cells was calculated as described by Balabanian et al. (2005). In order to obtain the relative migration in %, the percentage of migrated cells were normalized to the CXCL12-only control.

### Ca++ Signaling

For calcium measurement, 1×10 6 BCR-ABL-transformed murine bone marrow cells were incubated with 5 μg/mL of Indo-1 (Molecular Probes) and 0.5 μg/mL of pluronic F-127 (Molecular Probes) in Iscove's medium supplemented with 1% FCS (Pan Biotech) at 37° C. for 45 min. Cells were then washed by centrifugation and the cell pellets were resuspended in Iscove's medium with 1% FCS and treated with the EPI-X4 derivatives (1 μM or 0.5 μM) for 10 minutes at room temperature. Cells were pre-warmed for 5 minutes at 37° C. Calcium flux was assessed by FACS measurement at BD LSR Fortessa. After 30 seconds of baseline recording, CXCR4-dependent calcium signaling was determined by stimulating with 100 ng/ml of mouse SDF-1a (PeproTech).

### Molecular Modeling

The first step for the design of enhanced EPI-X4 (SEQ ID NO.: 1) derivatives was to determine how the peptide binds to CXCR4. With this knowledge, the inventors were able to improve ligand efficiency by designing shorter peptides that are potentially more active than EPI-X4 (SEQ ID NO.: 1). Accordingly, our computational approach comprised the following steps:

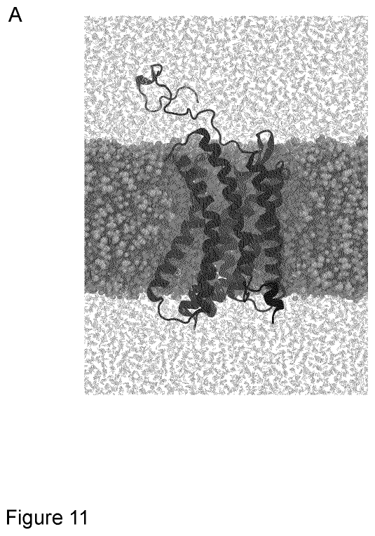

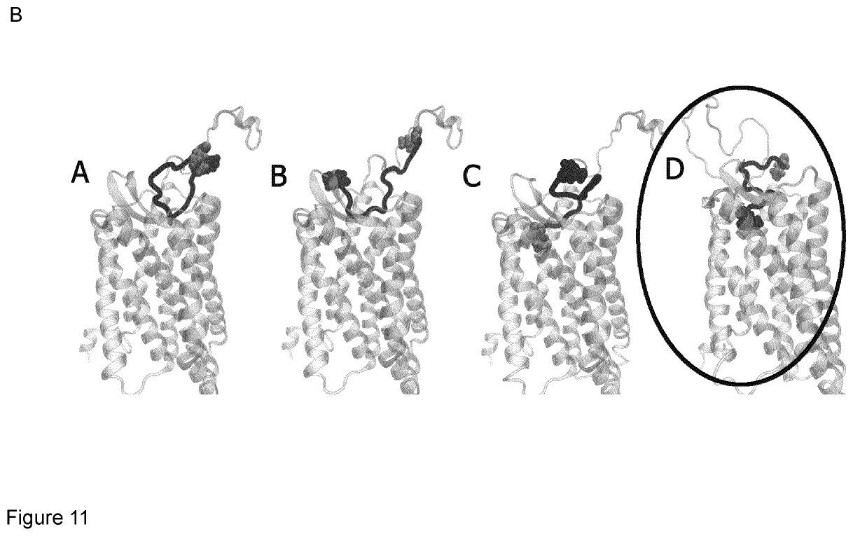

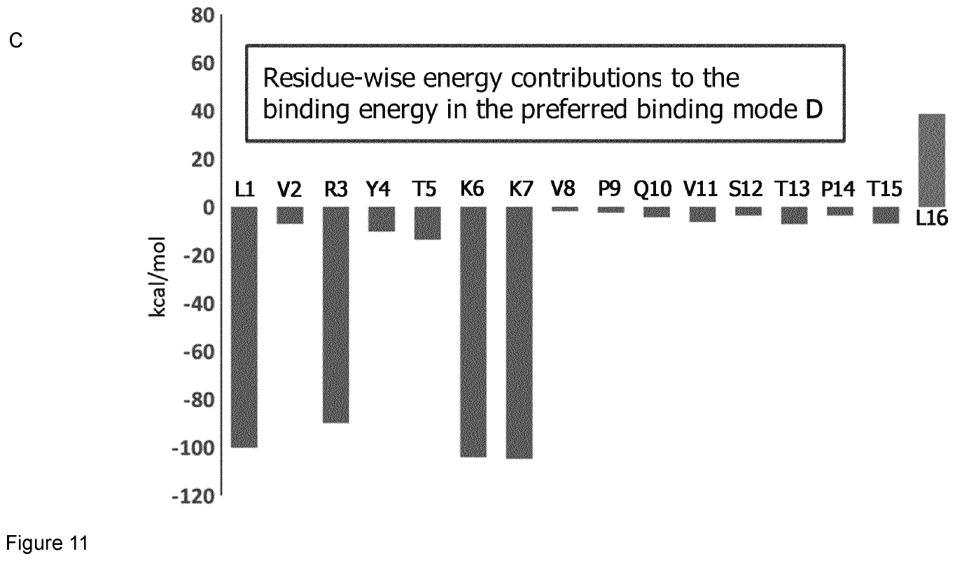

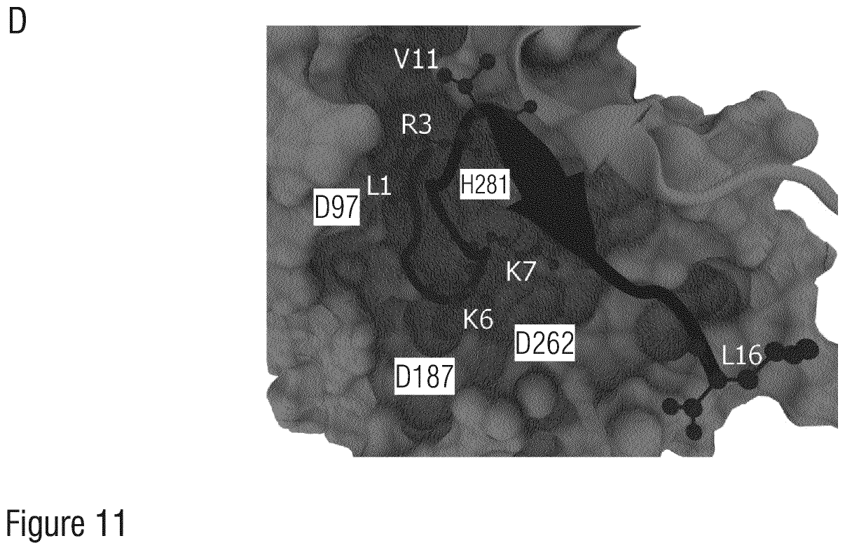

a. Building a CXCR4 model based on the reported crystal structure (2.50 Å, PDB code: 3ODU). This model also includes the highly flexible N-terminus region (available in the literature from NMR studies, PDB code 2K04).
b. Docking calculations and homology modeling for the initial exploration of EPI-X4 binding sites in CXCR4.
c. For each binding site, the inventors built CXCR4-EPI-X4 models in explicit solvent and lipid membrane (an example is shown in FIG. 11A). The models consisted of 257 POPC lipids, approximately 40000 TIP3P water molecules, 50 mM KCl and the CXCR4-EPI-X4 complex.
d. The inventors performed atomistic molecular dynamics simulations (MD) of each model to analyze factors like ligand flexibility, interaction interface area, solvent accessible surface and hydrogen bonding interactions in the different binding modes. The analysis of all these parameters indicated that D is the preferred binding motif. In D, the N-terminus of EPI-X4 is inserted in CXCR4 while the C-terminus of the peptide is solvent-exposed (FIG. 11 B).
e. Based on the MD simulations, the inventors performed an energetic analysis of the electrostatic and van der Waals contributions to the interaction energy in each binding motif as well as the contribution to the interaction energy of individual residues of EPI-X4 (FIG. 11 C).
f. The inventors also performed extensive coarse-grained (CG) MD simulations to investigate the self-assembly of the CXCR4-EPI-X4 complex from the unbound state, using non-equilibrium dynamics. With these CG simulations, we further established D as the most favored mode, as predicted by the atomistic MDs (FIG. 11D).

With the information of how EPI-X4 (SEQ ID NO.: 1) binds to CXCR4 and the individual contribution of each residue of the peptide to the binding, the inventors designed shortened peptide derivatives with neutral C-terminus that the inventors predict to be more efficient than EPI-X4 (SEQ ID NO.: 1). A set of peptides was thus identified and their experimental activity assessed.

Toxicity in Zebrafish. In order to test for toxic effects in zebrafish, fish embryos without chorion (24 hours post fertilization) were exposed to the compounds for 24 hours and then assayed in a stereomicroscope. Each assay was done for 3×10 embryos (in 100 μl) per concentration in duplicates (total n=60). As negative control peptide solvent at the highest concentration was used. As a positive control for acute toxicity pleurocidin antimicrobial peptide NRC-03 (GRRKRKWLRRIGKGVKIIGGAALDHL-NH2) (SEQ ID NO.: 103) at a concentration of 6 μM was used.

Polymer-coupled peptide synthesis. Materials 1,2-distearoyl-sn-glycero-3-phosphoethanolamine-N-[maleimide(polyethylene glycol) 2000 Da] (ammonium salt) (cat. No. PG2-DSML-2k) and cholesterol-poly (ethylene glycol) maleimide 5000 Da (PG2-CSML-5k) were purchased from Nanocs Inc. (New York, USA). Methoxy poly (ethylene glycol) 20 kDa maleimide (cat. No. PJK-231) and di-maleimide poly (ethylene glycol) 20 kDa (cat. No. PSB-305) were purchased from Creative PEGWorks (North Carolina, USA). All other chemicals were purchased from commercial vendors (Sigma-Aldrich, Acros, TCI). Deuterated solvents were supplied from Euriso-Top. Dichloromethane (CH2Cl2), acetonitrile (MeCN), tetrahydrofuran (THF) and toluene (PhMe) were acquired through a MBraun SPS-800 solvent purification system. Ultrapure water was dispensed from MilliQ Direct 8 (Millipore) [18.2 MΩ·cm].

¹H-NMR Spectroscopy Nuclear magnetic resonance (NMR) spectra were recorded on a Varian Mercury 400 MHz spectrometer, running at 400 MHz. Chemical shifts (δ) are reported in ppm relative to the residual solvent

Sodium Dodecyl Sulfate-Polyacrylamide Gel Electrophoresis (SDS PAGE) SDS-PAGE analysis was conducted with a NuPAGE® bis-tris 4-12% precast gel (Invitrogen) in an electrophoresis tank. Samples were prepared with NuPAGE® LDS sample buffer (4×). Samples volumes loaded were typically 10 μL. The running buffer was NuPAGE® MOPS SDS running buffer (20×). The voltage applied for electrophoresis was 150 V and the run time was 1 hour. The SDS PAGE gel was first stained with Coomassie blue stain for 30 minutes, then washed with distilled water for 1 hour.

Preparative Reverse-Phased High-Performance Liquid Chromatography (Prep RP-HPLC) RP-HPLC was conducted using C₁₈DiscoveryBIO Wide Pore column (10 μm, 150×10 mm) with 5% acetonitrile containing 0.01% trifluoracetic acid (TFA) as solvent A and 100% acetonitrile containing 0.01% TFA as solvent B. All solvents used were of HPLC grade. The gradient used was 30 to 60% solvent B in 25 minutes. Flow rate of 2 mL/min and UV detection at 280 nm was used for analysis.

Azeotropic distillation of PEG compounds Typically, to a flame-dried 50 mL schlenk flask fitted with a rubber septum and magnetic stir bar was added poly (ethylene glycol) (PEG) (0.05-0.1 g). Anhydrous toluene (5 mL) was injected into the flask using a clean glass syringe and needle. The flask was gently warmed to dissolve the PEG in toluene. The stoppered side arm of the schlenk flask was connected to a vacuum oil pump fitted with an ice trap. Toluene was observed to slowly foam upon slow opening of the side arm stopper to vacuum. The flask was gently swirled to avoid spluttering of the mixture. Moisture formed on the outer walls of the flask was wiped until all the solvent was removed from the flask. The flask was allowed to remain in vacuum for further 30 min at room temperature.

### Reagent Synthesis

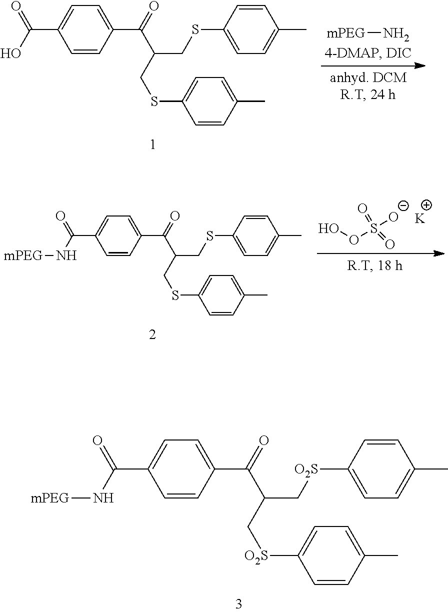

Synthesis of 4-(3-(p-tolylthio)-2-((p-tolylthio)methyl)propanoyl)benzoic acid. 4-(3-(p-Tolylthio)-2-((p-tolylthio)methyl)propanoyl)benzoic acid 1 was prepared as described previously in literature¹ (1.6 g, 72.7%). ¹H-NMR (CDCl₃): 2.38 (s, 6H), 3.16-3.31 (m, 4H), 3.85 (q, 1H), 7.15 (d, 4H), 7.18 (d, 4H), 7.64 (d, 2H), 8.07 (d, 2H)

Synthesis of bis-sulfide PEG 20 kDa. Toluene-dried methoxy poly (ethylene glycol) amine (mPEG-NH₂, 20000 g/mol, 100 mg, 1 equiv, 5.1 μmol) and 4-dimethylaminopyridine (0.06 mg, 0.1 equiv., 0.5 μmol) were dissolved in anhydrous dichloromethane (3 mL) under argon atmosphere. A mixture of 4-(3-(p-tolylthio)-2-((p-tolylthio)methyl)propanoyl)benzoic acid 1 (8.73 mg, 4 equiv, 20 μmol) and N,N′-diisopropylcarbodiimide (3.12 μL, 4 equiv, 200 μmol) in anhydrous dichloromethane (2 mL) was added drop-wise to the initial PEG solution under argon atmosphere. The reaction mixture was left to stir at room temperature for 24 h. After this time, dichloromethane was removed from the filtrate by roto-evaporation, and the viscous crude product residue was re-dissolved in acetone with gentle warming. The flask was then placed in a dry ice bath to precipitate the product, which was isolated by centrifugation dried in vacuo to afford PEG bis-sulfide 2 as a white solid product (0.101 g, 98.2%). ¹H NMR: (CDCl₃, 400 MHz) δ 2.49 (s, 6H), 3.38 (s, 3H), 3.44-3.84 (m, PEG+4H), 4.34 CHCO (qn, 1H), 7.36, 7.69 (q, 4H), 7.64, 7.81 (q, 4H).

Sulfide oxidation and synthesis of bis-sulfone PEG 20 kDa. Bis-sulfide PEG 20 kDa 2 (50 mg, 1 equiv., 2.5 μmol) and potassium peroxymonosulfate Oxone® (3.08 mg, 4 equiv., 10 μmol) were dissolved in an aqueous solution of 50% methanol (3 mL). The reaction mixture was stirred over-night at room temperature. After this time, volatiles were removed by rotary evaporation and purification achieved by acetone/dry-ice precipitation as described previously. The solid obtained was dried in desiccator to afford bis-sulfone PEG 3 as a white, fluffy solid (24 mg, 48%). ¹H-NMR (CDCl₃): 2.38 (s, 6H), 3.16-3.31 (m, 4H), 3.85 (q, 1H), 7.15 (d, 4H), 7.18 (d, 4H), 7.64 (d, 2H), 8.07 (d, 2H);

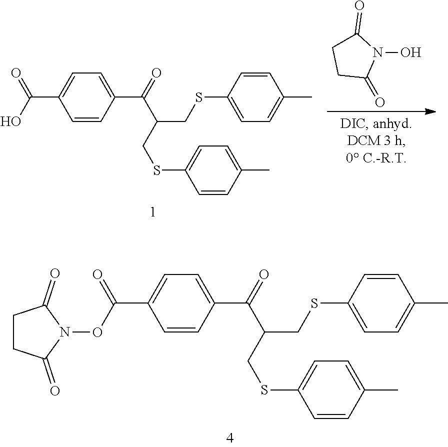

Synthesis of NHS-activated bis-sulfide 4. Under an argon atmosphere, a mixture of 4-(2,2-bis[(p-tolylsulfonyl)methyl]acetyl) benzoic acid 1 (0.5 g, 1 equiv., 1.15 mmol), N-hydroxysuccinimide (0.139 g, 1.05 equiv., 1.21 mmol) and anhydrous dichloromethane (5 mL) were cooled using an ice bath. Neat 1,3-diisopropylcarbodiimide (188 μL, 1.05 equiv., 1.21 mmol) was then added dropwise. A further 20 μL of DIC was added after 1.5 h. After 3 h, the reaction mixture was passed through a non-absorbent cotton wool filter. The homogeneous filtrate was diluted with dichloromethane, washed twice with water, and dried over magnesium sulphate. Gravity filtration followed by removal of volatiles under vacuum gave the desired active NHS ester 4 as a solid product (0.39 g, 78% yield). ¹H-NMR (CDCl₃): 2.35 (s, 6H), 2.94 (s, 4), 3.16-3.25 (dd, 4H), 3.80 (q, 1H), 7.05 (d, 4H), 7.10 (d, 4H), 7.60 (d, 2H), 8.05 (d, 2H);

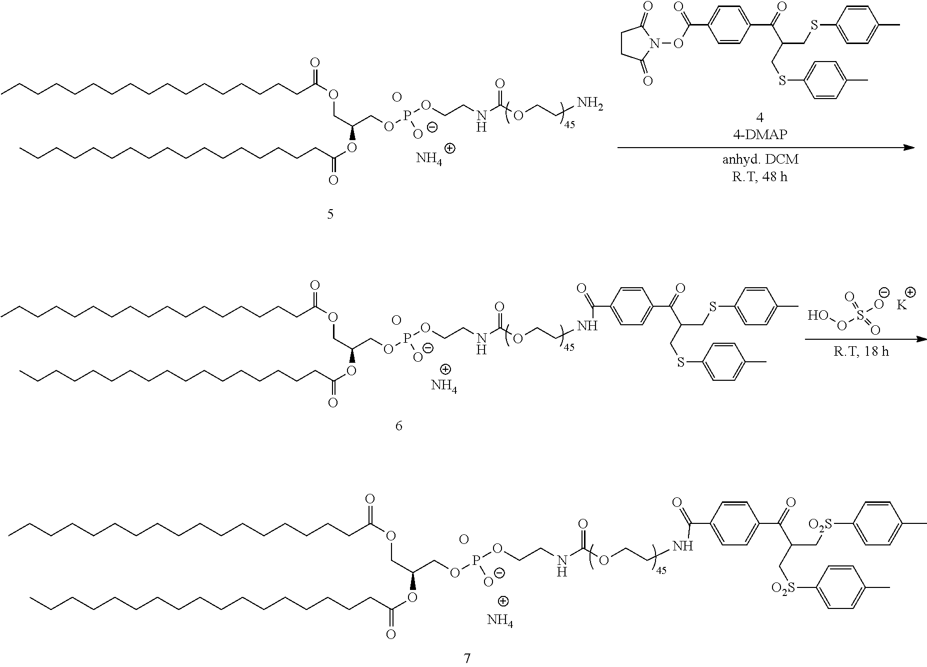

Synthesis of DSPE-PEG-bis-sulfide 2 kDa. Toluene-dried 1,2-distearoyl-sn-glycero-3-phosphoethanolamine-N-[amino(polyethylene glycol) 2 kDa] (ammonium salt) 5 (DSPE-PEG-NH₂, 2000 g/mol, 50 mg, 1 equiv, 25 μmol), NHS activated bis-sulfide 4 (53.4 mg, 4 equiv., 100 μmol), and 4-dimethylaminopyridine (0.3 mg, 0.1 equiv, 2.5 μmol) were dissolved in anhydrous dichloromethane (5 mL) under argon atmosphere. The reaction mixture was left to stir at room temperature for 48 h. After this time, dichloromethane was removed from the filtrate by rotary evaporation, and the viscous crude product residue was re-dissolved in acetone with gentle warming. The flask was then placed in a dry ice bath to precipitate the product, which was isolated by centrifugation dried in vacuo to afford DSPE-PEG bis-sulfide 6 as a white solid (26.5 mg, 42.4%). ¹H NMR: (CDCl₃, 400 MHz) δ 2.35 (s, 6H), 3.39-3.84 (m, PEG), 4.27 (br, 1H), 7.05 (d, 4H), 7.10 (d, 4H), 7.60 (d, 2H), 8.05 (d, 2H)

Sulfide oxidation to bis-sulfone DSPE-PEG 2 kDa. Bis-sulfide DSPE-PEG 2 kDa 5 (26.5 mg, 1 equiv., 8.28 μmol) and potassium peroxymonosulfate Oxone® (10.2 mg, 4 equiv., 33.1 μmol) were dissolved in an aqueous solution of 50% methanol (3 mL). The reaction mixture was stirred over-night at room temperature. After this time, volatiles were removed by rotary evaporation and purification achieved by acetone/dry-ice precipitation as described previously. The solid obtained was dried in desiccator to afford the bis-sulfone DSPE PEG 6 as a white solid (16.3 mg, 56.2%). ¹H-NMR (CDCl₃): 2.49 (s, 6H), 3.38-3.80 (m, PEG), 4.27 (m, 2H), 7.36 (d, 4H), 7.63 (d, 2H), 7.70 (d, 4H), 7.80 (d, 2H);

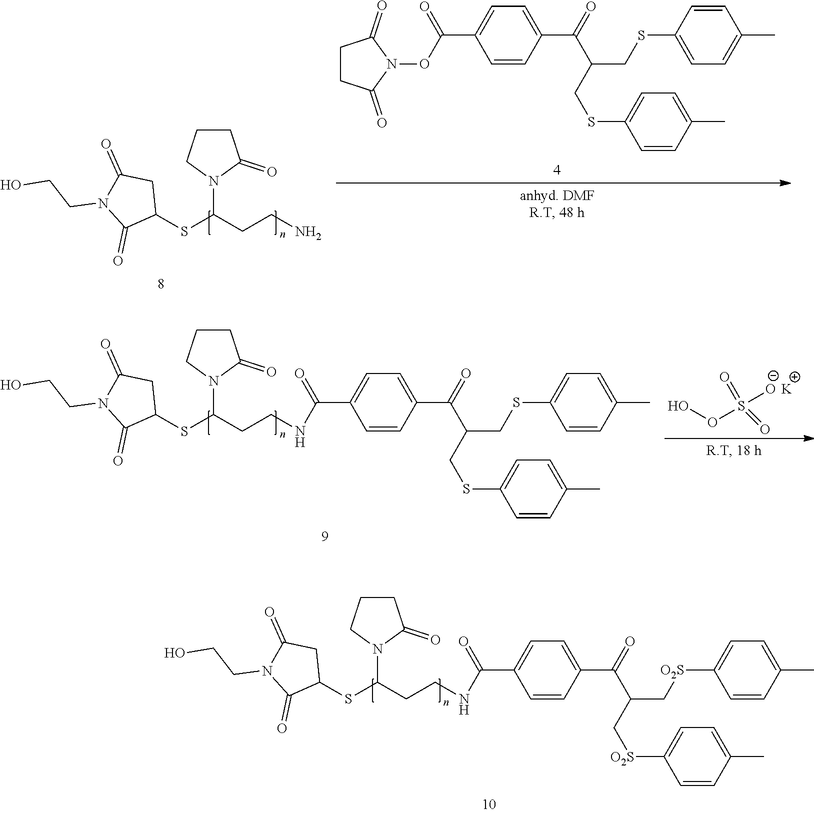

Synthesis of bis-sulfide PVP 20 kDa. Amine-terminated polyvinylpyrrolidone (PVP-NH₂, 23800 g/mol, 100 mg, 1 equiv, 4.2 μmol) and NHS-activated bis-sulfide (8.97 mg, 4 equiv, 16.8 μmol) were dissolved in anhydrous dimethylformamide (DMF, 2 mL) under argon atmosphere. The reaction mixture was stirred at room temperature for 48 h. After this time, the product was precipitated in ethyl ether and isolated by centrifugation. The obtained white precipitate was diluted in MQ water and lyophilized to afford bis-sulfide PVP as a white solid (66.5 mg, 66.5%). ¹H NMR: (CDCl₃, 400 MHz) 1.72 (br, 2H, PVP), 2.06 (br, 2H, PVP),), 2.39 (br, 2H, PVP), 3.20 (br, 2H, PVP), 3.73 (br, 1H, PVP), 7.04 (d, 4H), 7.08 (d, 4H), 7.5-7.6 (br, 4H)

Sulfide oxidation to bis-sulfone PVP 20 kDa. Bis-sulfide PVP 20 kDa 9 (66.5 mg, 1 equiv., 2.74 μmol) and potassium peroxymonosulfate Oxone® (5.07 mg, 4 equiv., 11 μmol) were dissolved in an aqueous solution of 50% methanol (3 mL). The reaction mixture was stirred over-night at room temperature. After this time, volatiles were removed by rotary evaporation and purification achieved by DMF/ethyl ether precipitation as described previously. The solid obtained was dried in desiccator to afford the bis-sulfone PVP as a white solid (31.85 mg, 47.7%). ¹H NMR: (CDCl₃, 400 MHz) 1.72 (br, 2H, PVP), 2.06 (br, 2H, PVP),), 2.39 (br, 2H, PVP+6H from bis-sulfone), 3.20 (br, 2H, PVP), 3.73 (br, 1H, PVP), 7.36 (d, 4H), 7.63 (d, 2H), 7.70 (d, 4H), 7.81 (d, 2H)

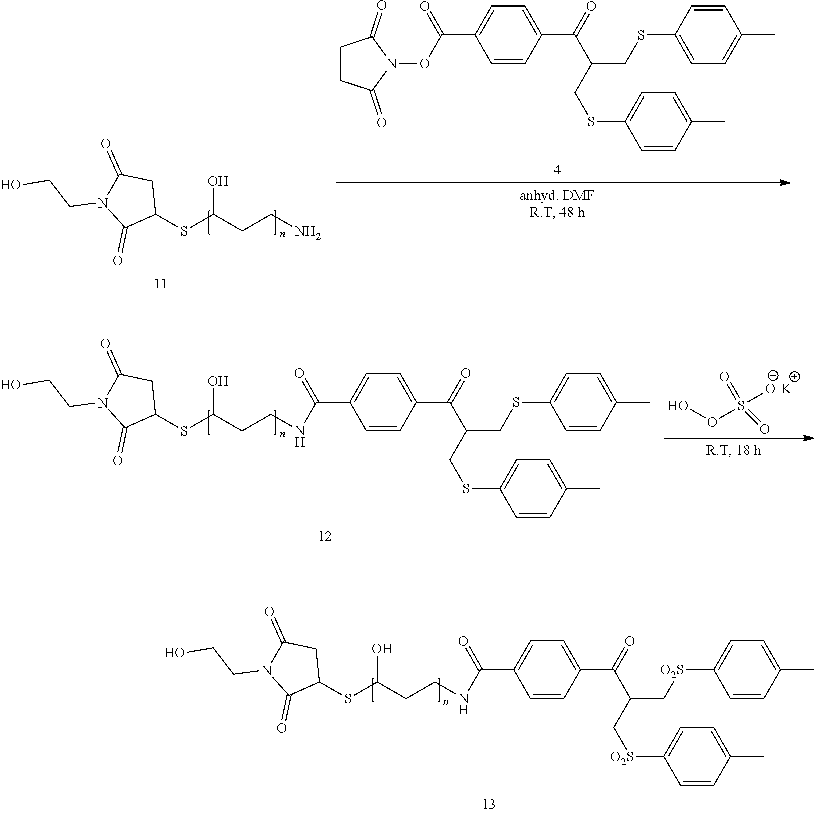

Synthesis of bis-sulfide PVA 20 kDa. Amine-terminated poly (vinyl alcohol) (PVA-NH₂, 19800 g/mol, 100 mg, 1 equiv, 4.2 μmol) was dissolved in DMSO (1 mL) and heated to 60° C. until complete dissolution. The solution was allowed to cool to room temperature and NHS-activated bis-sulfide (8.97 mg, 4 equiv, 16.8 μmol) in DMSO (1 mL) was added under argon atmosphere. The reaction mixture was stirred at room temperature for 48 h. After this time, the product was precipitated in heptane and isolated by centrifugation. The obtained white precipitate was diluted in MQ water and lyophilized to afford PVA bis-sulfide as a white solid (78.2 mg, 91.7%). ¹H NMR: (DMSO-d₆, 400 MHz) δ 1.36 (br, 2H, PVA), 3.81 (br, 1H, PVA), 7.04-7.10 (br, 8H), 7.5-7.6 (br, 4H)

Sulfide oxidation to bis-sulfone PVA 20 kDa. Bis-sulfide PVA 20 kDa (78.2 mg, 1 equiv., 3.85 μmol) and potassium peroxymonosulfate Oxone® (4.74 mg, 4 equiv., 15.4 μmol) were dissolved in an aqueous solution of 50% methanol (3 mL). The reaction mixture was stirred over-night at room temperature. After this time, volatiles were removed by rotary evaporation and purification achieved by DMSO/heptane precipitation as described previously. The solid obtained was dried in desiccator to afford the bis-sulfone PVA as a white solid (54.6 mg, 69.5%). ¹H NMR: (DMSO-d₆, 400 MHz) δ 1.36 (br, 2H, PVA), 3.81 (br, 1H, PVA), 7.34 (d, 4H), 7.62 (d, 2H), 7.70 (d, 4H), 7.80 (d, 2H)

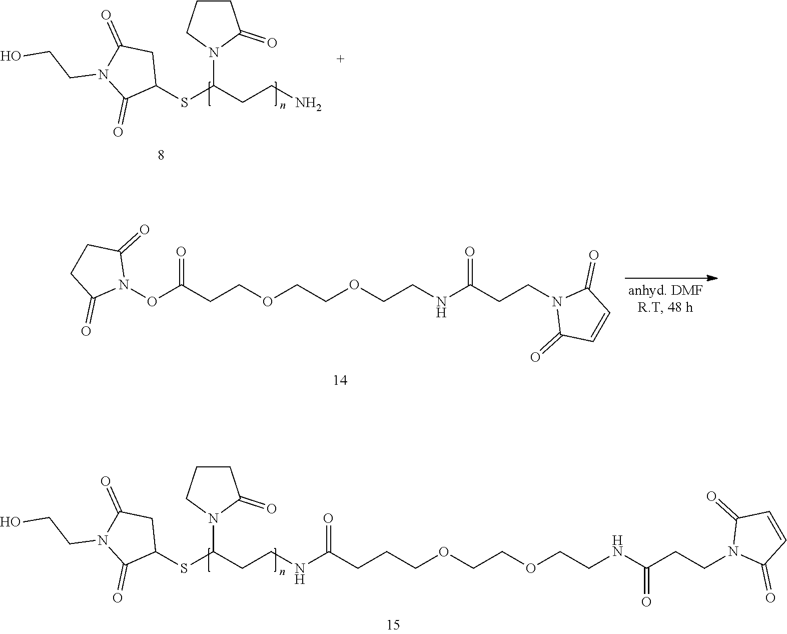

Synthesis of maleimide PVP 20 kDa. Amine-terminated polyvinylpyrrolidone (PVP-NH₂, 23800 g/mol, 50 mg, 1 equiv, 2.1 μmol) and maleimide-PEG₂-succinimidyl ester (3.57 mg, 4 equiv, 0.8 μmol) were dissolved in anhydrous dimethylformamide (DMF, 2 mL) under argon atmosphere. The reaction mixture was stirred at room temperature for 48 h. After this time, the product was precipitated in ethyl ether and isolated by centrifugation. The obtained white precipitate was diluted in MQ water and lyophilized to afford PVP maleimide as a white solid (23.6 mg, 47.2%). ¹H NMR: (CDCl₃, 400 MHz) 1.72 (br, 2H, PVP), 1.99 (br, 2H, PVP),), 2.38 (br, 2H, PVP), 3.20 (br, 2H, PVP), 3.73 (br, 1H, PVP), 8.02 (s, 2H).

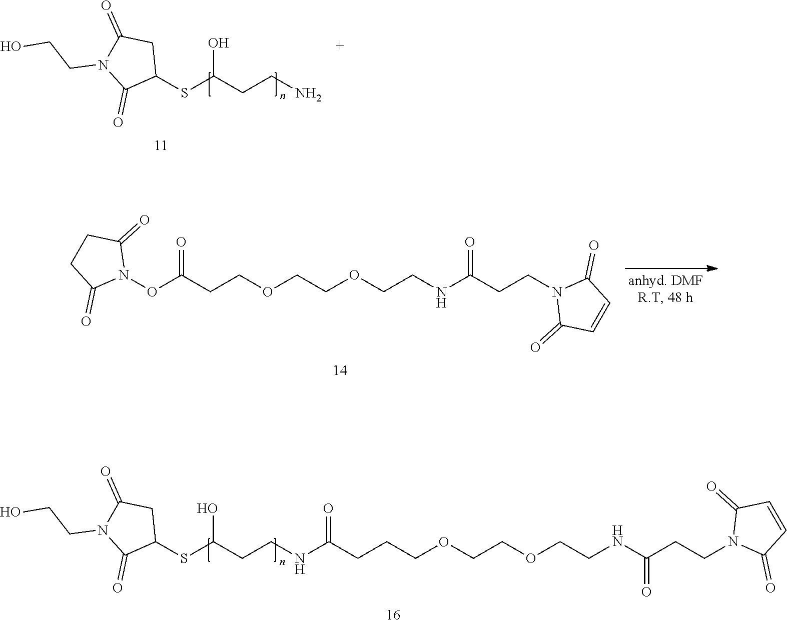

Synthesis of maleimide PVA 20 kDa. Amine-terminated polyvinylalcohol (PVÁ-NH₂, 19800 g/mol, 100 mg, 1 equiv, 5.05 μmol) was dissolved in anhydrous DMSO (2 mL) and heated to 60° C. After complete dissolution, maleimide-PEG₂-succinimidyl ester (8.59 mg, 4 equiv, 0.20.2 μmol) was added and the mixture was stirred at room temperature for 48 h. After this time, the product was precipitated in heptane and isolated by centrifugation. The obtained white precipitate was diluted in MQ water and lyophilized to afford PVA maleimide as a white solid (88.7 mg, 86.8%). ¹H NMR: (DMSO-d₆, 400 MHz) δ 1.36 (br, 2H, PVA), 3.81 (br, 1H, PVA), 6.99 (s, 2H, maleimide), 8.0 (br, 2H, —NH)

### Peptide Bioconjugation

General procedure for maleimide mono-conjugations. Native peptide (1 mg, 1 equiv., 0.714 μmol) was dissolved in 0.5 mL phosphate buffer saline, pH 7.4 (10 mM phosphate, 150 mM sodium chloride). To the peptide solution was added one equivalent of the respective maleimide conjugation reagent dissolved in 0.5 mL of PBS buffer, with a final peptide concentration of 1 mg/mL. The mixture was incubated for 4 h at room temperature. After this time, the bioconjugate was isolated from the native peptide by preparative RP-HPLC or gel filtration. The collected fractions were analysed by UV-Vis spectroscopy at 280 nm to determine the presence of peptide and combined respectively. After freeze-drying, the peptide conjugate was typically obtained as a solid.

TABLE 1
Quantities and purification methods for maleimide mono-conjugation
reactions

| | | Mass of | |
|---|---|---|---|
| Peptide | | Reagent | Purification |
| Conjugate | Conjugation Reagent | (mg) | method |
| DSPE- | DSPE-PEG₂₀₀₀-maleimide | 2.15 | Prep RP-HPLC |
| peptide | | | |
| Cholesterol- | Cholesterol-PEG₅₀₀₀- | 3.57 | LH20 gel filtration |
| peptide | maleimide | | |
| mPEG 5 | mPEG₅₀₀₀-maleimide | 3.57 | |
| kDa- | | | |
| peptide | | | |
| mPEG 20 | mPEG₂₀₀₀₀-maleimide | 14.8 | |
| kDa- | | | |
| peptide | | | |
| PVP 20 | PVP₂₀₀₀₀-maleimide | 17.4 | |
| kDa- | | | |
| peptide | | | |
| PVA 20 | PVA₂₀₀₀₀-maleimide | 14.3 | |
| kDa- | | | |
| peptide | | | |

General procedure for maleimide di-conjugation. Native peptide (10 mg, 1 equiv., 7.14 μmol) was dissolved in 5 mL phosphate buffer saline, pH 7.4 (10 mM phosphate, 150 mM sodium chloride). To the peptide solution was added di-maleimide PEG 20 kDa (81.4 mg, 0.5 equiv., 3.57 μmol) dissolved in 5 mL of PBS buffer. The mixture was incubated for 4 h at room temperature. After this time, the bioconjugate was isolated from the native peptide by LH20 gel filtration with ACN/MQ water as the eluent. The collected fractions were analysed by UV-Vis spectroscopy at 280 nm to determine the presence of peptide and combined respectively. After freeze-drying, the peptide conjugate was obtained as a solid.

General procedure for bis-sulfone conjugations. To one equivalent of the respective bis-alkylating reagent was added excess native peptide 10 equiv. in 1 mL of 50 mM sodium phosphate buffer, pH 7.8 with 20 mM EDTA. For PVA conjugations, the reagents were first dissolved in 100 μL of DMSO and heated to 60° C. to allow for dissolution. The mixture was incubated for 48 hours at room temperature. After this time, the bioconjugate was isolated from the native peptide by gel filtration. The collected fractions were analysed by UV-Vis spectroscopy at 280 nm to determine the presence of peptide and combined respectively. After freeze-drying, the peptide conjugate was typically obtained as a solid. Peptide content was characterized by UV absorbance, SDS-PAGE and/or RP-HPLC.

TABLE 2
Quantities and final peptide concentrations for bis-sulfone
conjugations

| Peptide | Conjugation | Reagent | Peptide | [Peptide]_{FINAL} |
|---|---|---|---|---|
| Conjugate | Reagent | (mg) | (mg) | mg/mL |
| DSPE-bis | DSPE-PEG₂₀₀₀-bis- | 5 | 21.8 | 10.9 |
| (peptide) | sulfone | | | |
| mPEG 20 | mPEG₂₀₀₀₀-bis- | 10 | 6.87 | 3.4 |
| kDa-bis | sulfone | | | |
| (peptide) | | | | |
| PVP 20 | PVP₂₀₀₀₀-bis-sulfone | 8.68 | 5 | 2 |
| kDa- b/s | | | | |
| (peptide) | | | | |
| PVA 20 | PVA₂₀₀₀₀-bis-sulfone | 7.25 | 5 | 2 |
| kDa-bis | | | | |
| (peptide) | | | | |

### Example

### Radioactive Labeling of DOTA-Conjugated Peptides and Evaluation of the Radiolabeled Conjugates

### DOTA-Conjugated Peptides

The following DOTA-conjugated peptides were used in this Example:

DOTA-K-JM #21 (SEQ ID NO.: 101, JM #206) (JM #21 (SEQ ID NO.: 23) with DOTA conjugated to the peptide via an additional lysine that is coupled to the C-terminal amino acid of the peptide),
DOTA-K-JM #122 (SEQ ID NO.: 102, JM #207) (JM #122 (SEQ ID NO.: 51) with DOTA conjugated to the peptide via an additional lysine that is coupled to the C-terminal amino acid of the peptide),
DOTA-K-JM #29 (SEQ ID NO.: 165) (JM #29 (SEQ ID NO.: 31) with DOTA conjugated to the peptide via an additional lysine that is coupled to the C-terminal amino acid of the peptide),
DOTA-JM #118 (SEQ ID NO.: 166) (JM #118 (SEQ ID NO.: 50) with DOTA conjugated to the peptide via the C-terminal lysine of the peptide),
DOTA-K-JM #173 (SEQ ID NO.: 169) (JM #173 (SEQ ID NO.: 70) with DOTA conjugated to the peptide via an additional lysine that is coupled to the C-terminal amino acid of the peptide), and
DOTA-K-JM #235 (SEQ ID NO.: 171) (JM #235 (SEQ ID NO.: 130) with DOTA conjugated to the peptide via an additional lysine that is coupled to the C-terminal amino acid of the peptide).

### Radiolabeling

¹⁷⁷Lu-labeled versions of DOTA-conjugated peptides were prepared in ammonium acetate buffer (0.4 M, pH 5.2) after incubation of 3 nmol of the peptide with different activities of [¹⁷⁷Lu]LuCl₃ (150-450 MBq). Ten % ethanol (except Pentixather) was added to the reaction mixture to prevent radiolysis at 75° C. for 30 min for cysteine-containing peptides and at 95° C. for 30 min for cysteine-free peptides. In addition, DTT (10 mM) was added to prevent the dimer formation for cysteine-containing peptides. For quality control 5 μl of this solution was added to 50 μl of Ca-DTPA solution and analysed via RP-HPLC. After determination of the radiochemical purity (>95%) the reaction mixture was diluted with human serum albumin (HSA) 1% to the desired activity concentration and used as such for evaluation.

⁶⁸Ga-labeled versions of DOTA-conjugated peptides were prepared in sodium acetate buffer (0.2 M, pH 4-4.5) after incubating 3 nmol of the peptide with different activities of [⁶⁸Ga]GaCl₃ (10-200 MBq) at 95° C. for 15 mins. For quality control 5 μl of this solution was added to 50 μl of Ca-DTPA solution and analysed via RP-HPLC. After determination of the radiochemical purity (>95%) the reaction mixture was diluted with human serum albumin (HSA) 1% to the desired activity concentration and used as such for evaluation.

### Stability

The stability of ¹⁷⁷Lu/⁶⁸Ga-labeled DOTA-conjugated peptides in ammonium acetate buffer (0.4 M, pH 5.2) and sodium acetate buffer (0.2 M, pH 4-4.5) was assessed by determining the radiochemical purity (RCP) of each radiolabeled conjugate at different time points (0, 1, 2, 4, and 24 h for ¹⁷⁷Lu-complexes and at 0, 1 and 2 h for ⁶⁸Ga-complexes) at room temperature. For this, an aliquot of labeling solution was stored at room temperature. RP-HPLC injections were consecutively performed at the desired time points.

Radiolysis-induced instability of [¹⁷⁷Lu]Lu-labeled DOTA-conjugated peptides over time was tracked by determining the radiochemical purity (Table 3). Results are means±standard deviation from a minimum of two separate experiments. At room temperature, the most stable were [¹⁷⁷Lu]Lu-DOTA-JM #118 with 80±2%, [¹⁷⁷Lu]Lu-DOTA-K-JM #235 with 80±10% and [¹⁷⁷Lu]Lu-DOTA-K-JM #207 with 78±1% of remaining radiolabeled conjugate after 24 h.

TABLE 3
Radiochemical purity of radiolabeled conjugates in NH₄-acetate
buffer pH 5.2 and radiolysis scavenger ethanol at different time points

| | Radiochemical purity [%] | | | | | |
|---|---|---|---|---|---|---|
| | | [¹⁷⁷Lu] | | | | |
| | | Lu- | | [¹⁷⁷Lu] | | |
| Time | [¹⁷⁷Lu]Lu- | DOTA- | [¹⁷⁷Lu]Lu- | Lu- | [¹⁷⁷Lu]Lu- | [¹⁷⁷Lu]Lu- |
| point | DOTA-K- | K- | DOTA-K- | DOTA- | DOTA-K- | DOTA-K- |
| [h] | JM#21 | JM#122 | JM#29 | JM#118 | JM#173 | JM#235 |
| 0 | 98 ± 0 | 97 ± 0 | 98 ± 1 | 99 ± 0 | 98 ± 1 | 96 ± 3 |
| 1 | 93 ± 3 | 97 ± 0 | 96 ± 3 | 98 ± 0 | 95 ± 0 | 96 ± 2 |
| 2 | 92 ± 6 | 96 ± 1 | 92 ± 9 | 97 ± 1 | 94 ± 0 | 94 ± 1 |
| 4 | 90 ± 7 | 96 ± 1 | 90 ± 9 | 95 ± 1 | 88 ± 3 | 93 ± 1 |
| 24 | 62 ± 13 | 78 ± 1 | n.d. | 80 ± 2 | 66 ± 0 | 80 ± 10 |

### Lipophilicity

The hydrophilic/lipophilic character of the ¹⁷⁷Lu/⁶⁸Ga-labeled conjugates was determined by the “shake-flask” method. To a pre-saturated solution containing 500 μL of n-octanol and 500 μL of phosphate-buffered saline (PBS) at pH 7.4, 10 μL of 1 picomol ¹⁷⁷Lu/⁶⁸Ga-labeled conjugates was added. The solutions were vortexed for 1 h to reach equilibrium and then centrifuged (3000 rpm) for 10 min. Hundred μl of the sample was removed from each phase and measured in a γ-counter. The partition coefficient was calculated as the average of the logarithmic values (n=3) of the ratio between the radioactivity in the organic and the PBS phase. Results are means±standard deviation from a minimum of two separate experiments. ¹⁷⁷Lu/⁶⁸Ga-labeled Pentixather, which is a known CXCR4-directed endoradiotherapeutic agent, is used as reference molecule.

Lipophilicity is an important physicochemical property of a potential radiotracer, playing a role in distribution in the body, excretion, pharmacokinetics and in plasma protein binding. When compared with the reference molecule [¹⁷⁷Lu]Lu-Pentixather (log D_{O/PBS pH7.4} −1.53±0.08), [¹⁷⁷Lu]Lu-DOTA-K-JM #122 shows the lowest log D_{O/PBS pH7.4} value −3.23±0.23, while [¹⁷⁷Lu]Lu-DOTA-K-JM #235 is the most lipophilic compound (log D_{O/PBS pH7.4} 0.29±0.10). The rest of the ¹⁷⁷Lu-labeled conjugates were found to have moderate lipophilicities (Table 4). In the case of ⁶⁸Ga-complexes, [⁶⁸Ga]Ga-Pentixather (log D_{O/PBS pH7.4} −2.17±0.07) was found to be more lipophilic compared to [⁶⁸Ga]Ga-DOTA-K-JM #173 (log D_{O/PBS pH7.4} −2.67±0.36) (Table 4).

TABLE 4
Lipophilicity of radiolabeled Pentixather and radiolabeled conjugates

| Radio ligand | log D_{O/PBS pH7.4} |
|---|---|
| [¹⁷⁷Lu]Lu-Pentixather | −1.53 ± 0.08 |
| [¹⁷⁷Lu]Lu-DOTA-K-JM#21 | −1.91 ± 0.24 |
| [¹⁷⁷Lu]Lu-DOTA-K-JM#122 | −3.23 ± 0.23 |
| [¹⁷⁷Lu]Lu-DOTA-K-JM#29 | −1.49 ± 0.29 |
| [¹⁷⁷Lu]Lu-DOTA-JM#118 | −3.12 ± 0.43 |
| [¹⁷⁷Lu]Lu-DOTA-K-JM#173 | −2.72 ± 0.22 |
| [¹⁷⁷Lu]Lu-DOTA-K-JM#235 | 0.29 ± 0.10 |
| [⁶⁸Ga]Ga-Pentixather | −2.17 ± 0.07 |
| [⁶⁸Ga]Ga-DOTA-K-JM#173 | −2.67 ± 0.36 |

### Cellular Uptake and Distribution

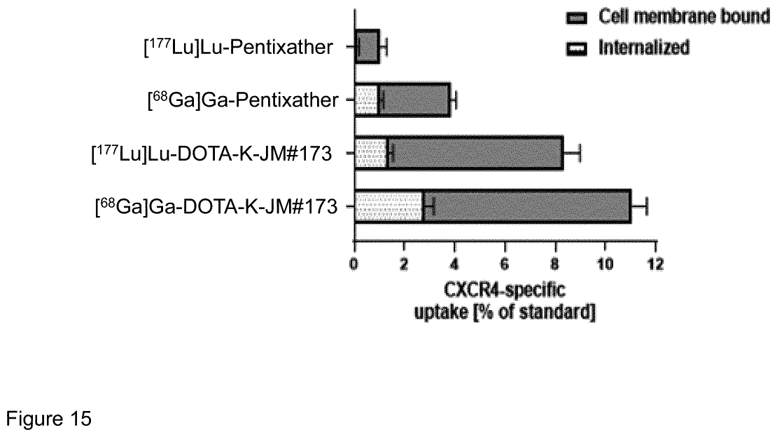

The receptor binding and internalization rates of ¹⁷⁷Lu/⁶⁸Ga-labeled conjugates were studied in GHOST-CXCR4+ cells seeded in 24-well plates (1×10⁵ cells/well). The radiolabeled conjugate (1 nM) was added and the cells were incubated at 37° C. for different time points (15, 30 and 60 min). Incubation was interrupted by the removal of the medium and washing the cells twice with ice-cold PBS. Membrane-bound radiolabeled conjugate was obtained by washing the cells twice with ice-cold glycine buffer pH 2.8, followed by collection of the internalized fraction with 1M NaOH. The activity in each fraction was measured in a γ-counter. Non-specific binding was determined in the presence of 100,000-fold excess of AMD3100 (blocking agent). The results are expressed as a percentage of the applied radioactivity and are demonstrated in FIG. 14 and FIG. 15, both showing the results for cellular uptake at 60 min.

[¹⁷⁷Lu]Lu-DOTA-K-JM #173 shows the highest overall cellular uptake as compared to all other ¹⁷⁷Lu-labeled conjugates (FIG. 14). More specifically, [¹⁷⁷Lu]Lu-DOTA-K-JM #173 is predominantly cell-membrane bound, while the more lipophilic [¹⁷⁷Lu]Lu-DOTA-K-JM #235 gets mainly internalized (FIG. 14). [¹⁷⁷Lu]Lu-DOTA-K-JM #173 displayed superiority over the reference molecule [¹⁷⁷Lu]Lu-Pentixather in this assay.

Based on the above results obtained for ¹⁷⁷Lu-labeled complexes, the best performing molecule (JM #173-K-DOTA (SEQ ID NO.: 169) was selected and evaluated in vitro using Ga-68. Even in this case, [⁶⁸Ga]Ga-DOTA-K-JM #173 exhibited higher cellular uptake when compared with [⁶⁸Ga]Ga-Pentixather (FIG. 15). [⁶⁸Ga]Ga-DOTA-K-JM #173 was seen to be 8.25±0.5% bound to the cell membrane (FIG. 15) and 2.82±0.3% internalized.

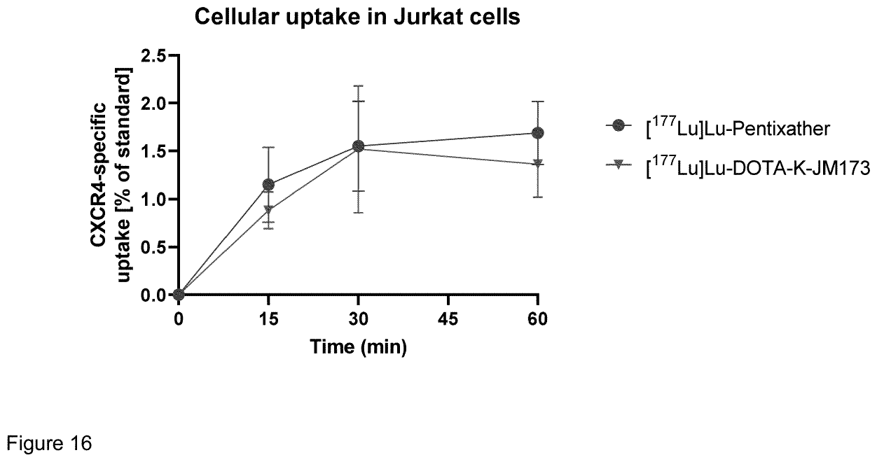

Next, in vitro assays in Jurkat cells (suspension), which had similar CXCR4 expression to that of GHOST-CXCR4+, were performed. Jurkat cells (4×10⁵ cells/sample) in assay medium containing 5% BSA were incubated with 1 nM of [¹⁷⁷Lu]Lu-Pentixather and [¹⁷⁷Lu]Lu-DOTA-K-JM #173 at 37° C. for different time points (15, 30 and 60 min) in presence and absence of AMD3100 (100 μM). Samples were then centrifuged, the supernatant was removed and the pellet was washed twice with 300 μL cold PBS. Finally, the supernatant and pellets were counted in the gamma counter to determine the total cellular uptake. Here the findings did not corroborate to that obtained with GHOST-CXCR4+ cells. In this study, both the compounds [¹⁷⁷Lu]Lu-Pentixather and [¹⁷⁷Lu]Lu-DOTA-K-JM #173 were found to have very similar cellular uptake in Jurkat cells (FIG. 16).

### Small Animal SPECT/CT and PET/CT Imaging

SPECT/CT: The total body distribution of [¹⁷⁷Lu]Lu-Pentixather was compared to [¹⁷⁷Lu]Lu-DOTA-K-JM #173 and [¹⁷⁷Lu]Lu-DOTA-K-JM #235. Healthy Balb/c mice were injected into the tail vein with 15-20 MBq (100 μmol) of ¹⁷⁷Lu-labeled complexes and SPECT/CT images was acquired 4 h post injection (p.i.). For acquiring the images, mice were euthanized by CO₂ inhalation after 4 hours, measured in a suitable dose calibrator and imaged supine, head first, using a SPECT/CT system dedicated to imaging small animals (NanoSPECT/CTTM Bioscan Inc.). The images were reconstructed using proprietary HiSPECT iterative reconstruction and fused with CT images using proprietary InVivoScope (Bioscan) software.

PET/CT: PET/CT imaging was performed to determine and compare the total body distribution of [⁶⁸Ga]Ga-Pentixather with [⁶⁸Ga]Ga-DOTA-K-JM #173. Healthy Balb/c mice were injected into the tail vein with 5-6 MBq (200 μmol) of ⁶⁸Ga-labeled complexes and PET/CT images were acquired 1 h p.i. For acquiring the images, mice were euthanized by CO₂ inhalation after 1 h, measured in a suitable dose calibrator and imaged supine, head first, using a PET/CT system dedicated to imaging small animals (Molecubes). The images were reconstructed using the Molecubes software and fused with CT images using Vivo Quant.

In order to get a first impression on in vivo characteristics of [¹⁷⁷Lu]Lu-DOTA-K-JM #173 and [⁶⁸Ga]Ga-DOTA-K-JM #173, a small animal nanoSPECT/CT and PET/CT imaging was performed, including Pentixather as a reference. The highly lipophilic compound [¹⁷⁷Lu]Lu-DOTA-K-JM #235 was also evaluated by SPECT/CT imaging to determine its distribution pattern. A distinct pharmacokinetic behavior was observed where [¹⁷⁷Lu]Lu-DOTA-K-JM #173, the less lipophilic compound (log D_{pH7.4}=−2.72±0.22), predominantly accumulates in kidneys, whereas the highly lipophilic [¹⁷⁷Lu]Lu-DOTA-K-JM #235 (log D_{pH7.4}=0.29±0.102) was seen to be predominantly accumulated in liver including some background activity. The reference compound Pentixather (log D_{pH7.4}=−1.53±0.08) displayed similar uptake in liver as [¹⁷⁷Lu]Lu-DOTA-K-JM #235, along with higher background activity.

### CONCLUSIONS

The radiolabeled DOTA-conjugated peptides have been evaluated in terms of lipophilicity, stability, and cellular uptake in GHOST-CXCR4+ cells. Out of the tested conjugates, [¹⁷⁷Lu]Lu-DOTA-K-JM #173 along with its diagnostic counterpart [⁶⁸Ga]Ga-DOTA-K-JM #173 is the most promising radiolabeled DOTA-conjugated peptide, as it exhibited the highest cellular uptake on GHOST-CXCR4+ cells compared to the other conjugates and the reference [¹⁷⁷Lu]Lu-Pentixather.

Further on, [¹⁷⁷Lu]Lu-DOTA-K-JM #173 and [⁶⁸Ga]Ga-DOTA-K-JM #173 showed no specific uptake in any organ in vivo. However, no uptake in other organs can also be attributed to the fact that these compounds are specific to human CXCR4. On the other hand, its renal accumulation is attributed to urinary excretion. Renal accumulation is preferable instead of hepatic accumulation as for [¹⁷⁷Lu]Lu-Pentixather. Renal uptake of radioactivity can be reduced by using nephroprotective agents, therefore lowering off-target radiotoxicity. Hepatic uptake instead, cannot be lowered and remains a major drawback for both, imaging and therapy. In this regard, [¹⁷⁷Lu]Lu-DOTA-K-JM #173 seems to be a suitable radiopharmaceutical.

Further disclosed are the following clauses:
1. A peptide consisting of one of the following amino acid sequences, selected from any of the groups 1 to 10, wherein
   Group 1 consists of

(SEQ ID NO.: 15, JM#13)
ILRWSRKMPCVS
(SEQ ID NO.: 19, JM#17)
IMRWSRKMPCVS
(SEQ ID NO.: 20, JM#18)
ILRWSRKMPCLS
(SEQ ID NO.: 22, JM#20)
ILRWSRKMPCMS
(SEQ ID NO.: 23, JM#21)
ILRWSRKLPCVS
(SEQ ID NO.: 24, JM#22)
ILRWSRKFPCVS
(SEQ ID NO.: 55, JM#144)
ILRWSRK(Pal)LPCVS
(SEQ ID NO.: 89, JM#192)
ILRWSRK(Glu-Pal)-NH2
(SEQ ID NO.: 91, JM#194)
d-LMRWSRK(Glu-Pal)-NH2

Group 2 consists of

(SEQ ID NO.: 21, JM#19)
ILRWSRKMPCFS
(SEQ ID NO.: 69, JM#172)
ILRWSRK(Pal)L-NH2
(SEQ ID NO.: 75, JM#178)
d-LMRWSRK(Pal)MP-NH2
(SEQ ID NO.: 77, JM#180)
d-LMRWSRK(Pal)-NH2
(SEQ ID NO.: 80, JM#183)
ILRWSRK(Ole)LPCVS

Group 3 consists of

(SEQ ID NO.: 53, JM#141)
d-LLRWSRK(Pal)MPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 66, JM#169)
IVRWSKK(Pal)VPCVS
(SEQ ID NO.: 67, JM#170)
ILRWSRK(Pal)-NH2
(SEQ ID NO.: 68, JM#171)
ILRWSRK(Pal)LP-NH2
(SEQ ID NO.: 88, JM#191)
ILRWSRKK(Glu-Pal)-NH2
(SEQ ID NO.: 90, JM#193)
ILRWSRKLK(Glu-Pal)-NH2
(SEQ ID NO.: 96, JM#199)
ILRWSRKK(Glu-Ste)LPCVS
(SEQ ID NO.: 98, JM#203)
ILRWSRK(Glu-Dec)LPCVS
(SEQ ID NO.: 100, JM#205)
ILRWSRK(Glu-Ste)LPCVS

Group 4 consists of

(SEQ ID NO.: 10, JM#8)
ILRWSRKVPCVS
(SEQ ID NO.: 12, JM#10)
IFRWSRKVPCVS
(SEQ ID NO.: 29, JM#27)
MLRWSRKMPCVS
(SEQ ID NO.: 36, JM#34)
MMRWSRKMPCVS
(SEQ ID NO.: 41, JM#39)
MLRWSRKLPCVS
(SEQ ID NO.: 51, JM#122)
ILRWSRKLPSVS
(SEQ ID NO.: 52, JM#140)
d-LLRWSRK(Glu-Pal)MPCVS
(SEQ ID NO.: 59, JM#149)
ILRWSRK(Pal)MPCLS
(SEQ ID NO.: 92, JM#195)
d-LMRWSRKK(Glu-Pal)-NH2
(SEQ ID NO.: 97, JM#200)
ILRWSRK-AcLPCVS

Group 5 consisting of

(SEQ ID NO.: 3, JM#1)
ILRWSKKVPCVS
(SEQ ID NO.: 4, JM#2)
IFRWSKKVPCVS
(SEQ ID NO.: 5, JM#3)
IVRWSRKVPCVS
(SEQ ID NO.: 6, JM#4)
IVRWSHKVPCVS
(SEQ ID NO.: 7, JM#5)
IVRWSKKLPCVS
(SEQ ID NO.: 8, JM#6)
IVRWSKKIPCVS
(SEQ ID NO.: 9, JM#7)
IVRWSKKFPCVS
(SEQ ID NO.: 11, JM#9)
ILRWSHKVPCVS
(SEQ ID NO.: 13, JM#11)
IFRWSHKVPCVS
(SEQ ID NO.: 14, JM#12)
IVRWSKKMPCVS
(SEQ ID NO.: 16, JM#14)
IVRWSKKVPCd-VS
(SEQ ID NO.: 17, JM#15)
ILRWSRKVPCd-VS
(SEQ ID NO.: 18, JM#16)
IIRWSRKMPCVS
(SEQ ID NO.: 25, JM#23)
ILRWSRKVPSVS
(SEQ ID NO.: 26, JM#24)
ILRWSRKMPSVS
(SEQ ID NO.: 27, JM#25)
Ac-SLRWSRKMPCVS
(SEQ ID NO.: 28, JM#26)
d-Ac-SLRWSRKMPCVS
(SEQ ID NO.: 30, JM#28)
d-MLRWSRKMPCVS
(SEQ ID NO.: 31, JM#29)
d-LLRWSRKMPCVS
(SEQ ID NO.: 32, JM#30)
d-FLRWSRKMPCVS
(SEQ ID NO.: 33, JM#31)
GLRWSRKMPCVS
(SEQ ID NO.: 34, JM#32)
Ac-SMRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 39, JM#37)
d-FMRWSRKMPCVS
(SEQ ID NO.: 40, JM#38)
d-GMRWSRKMPCVS
(SEQ ID NO.: 42, JM#40)
d-MLRWSRKLPCVS
(SEQ ID NO.: 43, JM#41)
Id-LRWSRKLPCVS
(SEQ ID NO.: 44, JM#42)
Id-LRWSRKMPCVS
(SEQ ID NO.: 45, JM#43)
Md-LRWSRKLPCVS
(SEQ ID NO.: 46, JM#44)
Md-LRWSRKMPCVS
(SEQ ID NO.: 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS
-Group 6 consists of
(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS

Group 7 consists of

(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 70, JM#173)
d-ILRWSRK-NH2
(SEQ ID NO.: 71, JM#174)
d-ILRWSRKLP-NH2

Group 8 consists of

(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2

Group 9 consists of

| | |
|---|---|
| Ac-SLRWSRKMPCVS | (SEQ ID NO.: 27, JM#25) |
| d-MLRWSRKMPCVS | (SEQ ID NO.: 30, JM#28) |
| d-Ac-SMRWSRKMPCVS | (SEQ ID NO.: 35, JM#33) |
| d-MMRWSRKMPCVS | (SEQ ID NO.: 37, JM#35) |
| d-LMRWSRKMPCVS | (SEQ ID NO.: 38, JM#36) |
| d-LLRWSRK(Glu-Pal)MPCVS | (SEQ ID NO.: 52, JM#140) |
| d-LMRWSRK(Pal)MP-NH2 | (SEQ ID NO.: 75, JM#178) |

Group 10 consists of

| | |
|---|---|
| ILRWSRK(Pal)LPCVS | (SEQ ID NO.: 55, JM#144) |
| IVRWSK(Pal)KVP-NH2 | (SEQ ID NO.: 63, JM#166) |
| IVRWSKK(Pal)VP-NH2 | (SEQ ID NO.: 64, JM#167) |
| ILRWSRK(Pal)-NH2 | (SEQ ID NO.: 67, JM#170) |
| ILRWSRK(Pal)LP-NH2 | (SEQ ID NO.: 68, JM#171) |
| ILRWSRK(Pal)L-NH2 | (SEQ ID NO.: 69, JM#172) |
| d-LMRWSRK(Pal)-NH2 | (SEQ ID NO.: 77, JM#180) |
| ILRWSRKK(Glu-Pal)-NH2 | (SEQ ID NO.: 88, JM#191) |
| ILRWSRK(Glu-Pal)-NH2 | (SEQ ID NO.: 89, JM#192) |
| ILRWSRKLK(Glu-Pal)-NH2 | (SEQ ID NO.: 90, JM#193) |
| d-LMRWSRK(Glu-Pal)-NH2 | (SEQ ID NO.: 91, JM#194) |

wherein d- in front of an amino acid indicates a D-Amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol, and Ac indicates a substitution of an amino group by an acetyl group.
2. A peptide consisting of one of the following amino acid sequences, selected from any of the groups 1 to 10, wherein
   Group 1 consists of

| | |
|---|---|
| ILRWSRKMPCVS | (SEQ ID NO.: 15, JM#13) |
| IMRWSRKMPCVS | (SEQ ID NO.: 19, JM#17) |
| ILRWSRKMPCLS | (SEQ ID NO.: 20, JM#18) |
| ILRWSRKMPCMS | (SEQ ID NO.: 22, JM#20) |
| ILRWSRKLPCVS | (SEQ ID NO.: 23, JM#21) |
| ILRWSRKFPCVS | (SEQ ID NO.: 24, JM#22) |
| ILRWSRK(Pal)LPCVS | (SEQ ID NO.: 55, JM#144) |
| ILRWSRK(Glu-Pal)-NH2 | (SEQ ID NO.: 89, JM#192) |
| d-LMRWSRK(Glu-Pal)-NH2 | (SEQ ID NO.: 91, JM#194) |

Group 2 consists of

| | |
|---|---|
| ILRWSRKMPCFS | (SEQ ID NO.: 21, JM#19) |
| ILRWSRK(Pal)L-NH2 | (SEQ ID NO.: 69, JM#172) |
| d-LMRWSRK(Pal)MP-NH2 | (SEQ ID NO.: 75, JM#178) |
| d-LMRWSRK(Pal)-NH2 | (SEQ ID NO.: 77, JM#180) |
| ILRWSRK(Ole)LPCVS | (SEQ ID NO.: 80, JM#183) |

Group 3 consists of

| | |
|---|---|
| d-LLRWSRK(Pal)MPCVS | (SEQ ID NO.: 53, JM#141) |
| IVRWSK(Pal)KVP-NH2 | (SEQ ID NO.: 63, JM#166) |
| IVRWSKK(Pal)VP-NH2 | (SEQ ID NO.: 64, JM#167) |
| IVRWSKK(Pal)VPCVS | (SEQ ID NO.: 66, JM#169) |
| ILRWSRK(Pal)-NH2 | (SEQ ID NO.: 67, JM#170) |
| ILRWSRK(Pal)LP-NH2 | (SEQ ID NO.: 68, JM#171) |
| ILRWSRKK(Glu-Pal)-NH2 | (SEQ ID NO.: 88, JM#191) |
| ILRWSRKLK(Glu-Pal)-NH2 | (SEQ ID NO.: 90, JM#193) |
| ILRWSRKK(Glu-Ste)LPCVS | (SEQ ID NO.: 96, JM#199) |
| ILRWSRK(Glu-Dec)LPCVS | (SEQ ID NO.: 98, JM#203) |
| ILRWSRK(Glu-Ste)LPCVS | (SEQ ID NO.: 100, JM#205) |

Group 4 consists of

| | |
|---|---|
| ILRWSRKVPCVS | (SEQ ID NO.: 10, JM#8) |
| IFRWSRKVPCVS | (SEQ ID NO.: 12, JM#10) |
| MLRWSRKMPCVS | (SEQ ID NO.: 29, JM#27) |
| MMRWSRKMPCVS | (SEQ ID NO.: 36, JM#34) |
| MLRWSRKLPCVS | (SEQ ID NO.: 41, JM#39) |
| ILRWSRKLPSVS | (SEQ ID NO.: 51, JM#122) |
| d-LLRWSRK(Glu-Pal)MPCVS | (SEQ ID NO.: 52, JM#140) |
| ILRWSRK(Pal)MPCLS | (SEQ ID NO.: 59, JM#149) |
| d-LMRWSRKK(Glu-Pal)-NH2 | (SEQ ID NO.: 92, JM#195) |
| ILRWSRK-AcLPCVS | (SEQ ID NO.: 97, JM#200) |

Group 5 consisting of

| | |
|---|---|
| ILRWSKKVPCVS | (SEQ ID NO.: 3, JM#1) |
| IFRWSKKVPCVS | (SEQ ID NO.: 4, JM#2) |
| IVRWSRKVPCVS | (SEQ ID NO.: 5, JM#3) |
| IVRWSHKVPCVS | (SEQ ID NO.: 6, JM#4) |
| IVRWSKKLPCVS | (SEQ ID NO.: 7, JM#5) |
| IVRWSKKIPCVS | (SEQ ID NO.: 8, JM#6) |
| IVRWSKKFPCVS | (SEQ ID NO.: 9, JM#7) |
| ILRWSHKVPCVS | (SEQ ID NO.: 11, JM#9) |
| IFRWSHKVPCVS | (SEQ ID NO.: 13, JM#11) |
| IVRWSKKMPCVS | (SEQ ID NO.: 14, JM#12) |
| IVRWSKKVPCd-VS | (SEQ ID NO.: 16, JM#14) |
| ILRWSRKVPCd-VS | (SEQ ID NO.: 17, JM#15) |
| IIRWSRKMPCVS | (SEQ ID NO.: 18, JM#16) |
| ILRWSRKVPSVS | (SEQ ID NO.: 25, JM#23) |
| ILRWSRKMPSVS | (SEQ ID NO.: 26, JM#24) |
| Ac-SLRWSRKMPCVS | (SEQ ID NO.: 27, JM#25) |
| d-Ac-SLRWSRKMPCVS | (SEQ ID NO.: 28, JM#26) |
| d-MLRWSRKMPCVS | (SEQ ID NO.: 30, JM#28) |
| d-LLRWSRKMPCVS | (SEQ ID NO.: 31, JM#29) |
| d-FLRWSRKMPCVS | (SEQ ID NO.: 32, JM#30) |
| GLRWSRKMPCVS | (SEQ ID NO.: 33, JM#31) |
| Ac-SMRWSRKMPCVS | (SEQ ID NO.: 34, JM#32) |
| d-Ac-SMRWSRKMPCVS | (SEQ ID NO.: 35, JM#33) |
| d-MMRWSRKMPCVS | (SEQ ID NO.: 37, JM#35) |
| d-LMRWSRKMPCVS | (SEQ ID NO.: 38, JM#36) |
| d-FMRWSRKMPCVS | (SEQ ID NO.: 39, JM#37) |
| d-GMRWSRKMPCVS | (SEQ ID NO.: 40, JM#38) |
| d-MLRWSRKLPCVS | (SEQ ID NO.: 42, JM#40) |
| Id-LRWSRKLPCVS | (SEQ ID NO.: 43, JM#41) |
| Id-LRWSRKMPCVS | (SEQ ID NO.: 44, JM#42) |
| Md-LRWSRKLPCVS | (SEQ ID NO.: 45, JM#43) |
| Md-LRWSRKMPCVS | (SEQ ID NO.: 46, JM#44) |
| IVRWSKKVP-NH2 | (SEQ ID NO.: 47, JM#106) |
| IVRWSKK-NH2 | (SEQ ID NO.: 48, JM#110) |
| ILRWSRKLP-NH2 | (SEQ ID NO.: 49, JM#114) |
| ILRWSRK-NH2 | (SEQ ID NO.: 50, JM#118) |
| ILRWSRK(Glu-Pal)LPCVS | (SEQ ID NO.: 54, JM#143) |
| ILRWSRKLPCK(Glu-Pal)S | (SEQ ID NO.: 56, JM#145) |
| IYRWSRKMPCLS | (SEQ ID NO.: 57, JM#146) |
| ILRWSRK(Glu-Pal)MPCLS | (SEQ ID NO.: 58, JM#148) |
| IVRWSKKVPSVS | (SEQ ID NO.: 60, JM#151) |
| IVRWSK(Pal)K-NH2 | (SEQ ID NO.: 61, JM#164) |
| IVRWSKK(Pal)-NH2 | (SEQ ID NO.: 62, JM#165) |
| IVRWSK(Pal)KVPCVS | (SEQ ID NO.: 65, JM#168) |
| d-ILRWSRK-NH2 | (SEQ ID NO.: 70, JM#173) |
| d-ILRWSRKLP-NH2 | (SEQ ID NO.: 71, JM#174) |
| d-ILRWSRK(Pal)LP-NH2 | (SEQ ID NO.: 72, JM#175) |
| d-LMRWSRK(Pal)MPCVS | (SEQ ID NO.: 73, JM#176) |
| Md-LRWSRK(Pal)LPCVS | (SEQ ID NO.: 74, JM#177) |
| Md-LRWSRK(Pal)LP-NH2 | (SEQ ID NO.: 76, JM#179) |
| Md-LRWSRK(Pal)-NH2 | (SEQ ID NO.: 78, JM#181) |
| ILRWSRK(Ste)LPCVS | (SEQ ID NO.: 79, JM#182) |
| ILRWSRK(Chl)LPCVS | (SEQ ID NO.: 81, JM#184) |
| Ac-ILRWSRKLPCVS | (SEQ ID NO.: 82, JM#185) |
| d-Ac-ILRWSRKLPCVS | (SEQ ID NO.: 83, JM#186) |
| Ac-MLRWSRKLPCVS | (SEQ ID NO.: 84, JM#187) |
| d-Ac-MLRWSRKLPCVS | (SEQ ID NO.: 85, JM#188) |
| VLRWSRKLPCVS | (SEQ ID NO.: 86, JM#189) |
| d-VLRWSRKLPCVS | (SEQ ID NO.: 87, JM#190) |
| d-MLRWSRK(Glu-Pal)-NH2 | (SEQ ID NO.: 93, JM#196) |
| d-MLRWSRKK(Glu-Pal)-NH2 | (SEQ ID NO.: 94, JM#197) |
| ILRWSRK(Glu-Ste)LPCVS | (SEQ ID NO.: 95, JM#198) |
| ILRWSRK(Glu-Myr)LPCVS | (SEQ ID NO.: 99, JM#204) |
| Group 6 consists of | |
| d-LMRWSRKK(Glu-Pal)-NH2 | (SEQ ID NO.: 92, JM#195) |
| d-MLRWSRK(Glu-Pal)-NH2 | (SEQ ID NO.: 93, JM#196) |
| d-MLRWSRKK(Glu-Pal)-NH2 | (SEQ ID NO.: 94, JM#197) |
| ILRWSRK(Glu-Ste)LPCVS | (SEQ ID NO.: 95, JM#198) |

Group 7 consists of

| | |
|---|---|
| Md-LRWSRKLPCVS | (SEQ ID NO.: 45, JM#43) |
| Md-LRWSRKMPCVS | (SEQ ID NO.: 46, JM#44) |
| ILRWSRK(Glu-Pal)LPCVS | (SEQ ID NO.: 54, JM#143) |
| ILRWSRK(Pal)LPCVS | (SEQ ID NO.: 55, JM#144) |
| ILRWSRKLPCK(Glu-Pal)S | (SEQ ID NO.: 56, JM#145) |
| ILRWSRK(Pal)MPCLS | (SEQ ID NO.: 59, JM#149) |
| IVRWSK(Pal)KVP-NH2 | (SEQ ID NO.: 63, JM#166) |
| IVRWSKK(Pal)VP-NH2 | (SEQ ID NO.: 64, JM#167) |
| ILRWSRK(Pal)-NH2 | (SEQ ID NO.: 67, JM#170) |
| ILRWSRK(Pal)LP-NH2 | (SEQ ID NO.: 68, JM#171) |
| d-ILRWSRK-NH2 | (SEQ ID NO.: 70, JM#173) |
| d-ILRWSRKLP-NH2 | (SEQ ID NO.: 71, JM#174) |

Group 8 consists of

| | |
|---|---|
| d-LLRWSRKMPCVS | (SEQ ID NO.: 31, JM#29) |
| ILRWSRK(Pal)L-NH2 | (SEQ ID NO.: 69, JM#172) |
| ILRWSRK(Glu-Pal)-NH2 | (SEQ ID NO.: 89, JM#192) |

Group 9 consists of

| | |
|---|---|
| Ac-SLRWSRKMPCVS | (SEQ ID NO.: 27, JM#25) |
| d-MLRWSRKMPCVS | (SEQ ID NO.: 30, JM#28) |
| d-Ac-SMRWSRKMPCVS | (SEQ ID NO.: 35, JM#33) |
| d-MMRWSRKMPCVS | (SEQ ID NO.: 37, JM#35) |
| d-LMRWSRKMPCVS | (SEQ ID NO.: 38, JM#36) |
| d-LLRWSRK(Glu-Pal)MPCVS | (SEQ ID NO.: 52, JM#140) |
| d-LMRWSRK(Pal)MP-NH2 | (SEQ ID NO.: 75, JM#178) |

Group 10 consists of

| | |
|---|---|
| ILRWSRK(Pal)LPCVS | (SEQ ID NO.: 55, JM#144) |
| IVRWSK(Pal)KVP-NH2 | (SEQ ID NO.: 63, JM#166) |
| IVRWSKK(Pal)VP-NH2 | (SEQ ID NO.: 64, JM#167) |
| ILRWSRK(Pal)-NH2 | (SEQ ID NO.: 67, JM#170) |
| ILRWSRK(Pal)LP-NH2 | (SEQ ID NO.: 68, JM#171) |
| ILRWSRK(Pal)L-NH2 | (SEQ ID NO.: 69, JM#172) |
| d-LMRWSRK(Pal)-NH2 | (SEQ ID NO.: 77, JM#180) |
| ILRWSRKK(Glu-Pal)-NH2 | (SEQ ID NO.: 88, JM#191) |
| ILRWSRK(Glu-Pal)-NH2 | (SEQ ID NO.: 89, JM#192) |
| ILRWSRKLK(Glu-Pal)-NH2 | (SEQ ID NO.: 90, JM#193) |
| d-LMRWSRK(Glu-Pal)-NH2 | (SEQ ID NO.: 91, JM#194) |

wherein d- in front of an amino acid indicates a D-Amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol, and Ac indicates a substitution of an amino group by an acetyl group, and wherein the peptide is C-terminally conjugated to a complexing agent.
3. A peptide consisting of one of the following amino acid sequences, selected from any of the groups 1 to 10, wherein
   Group 1 consists of

| | |
|---|---|
| ILRWSRKMPCVS | (SEQ ID NO.: 15, JM#13) |
| IMRWSRKMPCVS | (SEQ ID NO.: 19, JM#17) |
| ILRWSRKMPCLS | (SEQ ID NO.: 20, JM#18) |
| ILRWSRKMPCMS | (SEQ ID NO.: 22, JM#20) |
| ILRWSRKLPCVS | (SEQ ID NO.: 23, JM#21) |
| ILRWSRKFPCVS | (SEQ ID NO.: 24, JM#22) |
| ILRWSRK(Pal)LPCVS | (SEQ ID NO.: 55, JM#144) |
| ILRWSRK(Glu-Pal)-NH2 | (SEQ ID NO.: 89, JM#192) |
| d-LMRWSRK(Glu-Pal)-NH2 | (SEQ ID NO.: 91, JM#194) |

Group 2 consists of

| | |
|---|---|
| ILRWSRKMPCFS | (SEQ ID NO.: 21, JM#19) |
| ILRWSRK(Pal)L-NH2 | (SEQ ID NO.: 69, JM#172) |
| d-LMRWSRK(Pal)MP-NH2 | (SEQ ID NO.: 75, JM#178) |
| d-LMRWSRK(Pal)-NH2 | (SEQ ID NO.: 77, JM#180) |
| ILRWSRK(Ole)LPCVS | (SEQ ID NO.: 80, JM#183) |

Group 3 consists of

| | |
|---|---|
| d-LLRWSRK(Pal)MPCVS | (SEQ ID NO.: 53, JM#141) |
| IVRWSK(Pal)KVP-NH2 | (SEQ ID NO.: 63, JM#166) |
| IVRWSKK(Pal)VP-NH2 | (SEQ ID NO.: 64, JM#167) |
| IVRWSKK(Pal)VPCVS | (SEQ ID NO.: 66, JM#169) |
| ILRWSRK(Pal)-NH2 | (SEQ ID NO.: 67, JM#170) |
| ILRWSRK(Pal)LP-NH2 | (SEQ ID NO.: 68, JM#171) |
| ILRWSRKK(Glu-Pal)-NH2 | (SEQ ID NO.: 88, JM#191) |
| ILRWSRKLK(Glu-Pal)-NH2 | (SEQ ID NO.: 90, JM#193) |
| ILRWSRKK(Glu-Ste)LPCVS | (SEQ ID NO.: 96, JM#199) |
| ILRWSRK(Glu-Dec)LPCVS | (SEQ ID NO.: 98, JM#203) |
| ILRWSRK(Glu-Ste)LPCVS | (SEQ ID NO.: 100, JM#205) |

Group 4 consists of

| | |
|---|---|
| ILRWSRKVPCVS | (SEQ ID NO.: 10, JM#8) |
| IFRWSRKVPCVS | (SEQ ID NO.: 12, JM#10) |
| MLRWSRKMPCVS | (SEQ ID NO.: 29, JM#27) |
| MMRWSRKMPCVS | (SEQ ID NO.: 36, JM#34) |
| MLRWSRKLPCVS | (SEQ ID NO.: 41, JM#39) |
| ILRWSRKLPSVS | (SEQ ID NO.: 51, JM#122) |
| d-LLRWSRK(Glu-Pal)MPCVS | (SEQ ID NO.: 52, JM#140) |
| ILRWSRK(Pal)MPCLS | (SEQ ID NO.: 59, JM#149) |
| d-LMRWSRKK(Glu-Pal)-NH2 | (SEQ ID NO.: 92, JM#195) |
| ILRWSRK-AcLPCVS | (SEQ ID NO.: 97, JM#200) |

Group 5 consisting of

(SEQ ID NO.: 3, JM#1)
ILRWSKKVPCVS
(SEQ ID NO.: 4, JM#2)
IFRWSKKVPCVS
(SEQ ID NO.: 5, JM#3)
IVRWSRKVPCVS
(SEQ ID NO.: 6, JM#4)
IVRWSHKVPCVS
(SEQ ID NO.: 7, JM#5)
IVRWSKKLPCVS
(SEQ ID NO.: 8, JM#6)
IVRWSKKIPCVS
(SEQ ID NO.: 9, JM#7)
IVRWSKKFPCVS
(SEQ ID NO.: 11, JM#9)
ILRWSHKVPCVS
(SEQ ID NO.: 13, JM#11)
IFRWSHKVPCVS
(SEQ ID NO.: 14, JM#12)
IVRWSKKMPCVS
(SEQ ID NO.: 16, JM#14)
IVRWSKKVPCd-VS
(SEQ ID NO.: 17, JM#15)
ILRWSRKVPCd-VS
(SEQ ID NO.: 18, JM#16)
IIRWSRKMPCVS
(SEQ ID NO.: 25, JM#23)
ILRWSRKVPSVS
(SEQ ID NO.: 26, JM#24)
ILRWSRKMPSVS
(SEQ ID NO.: 27, JM#25)
Ac-SLRWSRKMPCVS
(SEQ ID NO.: 28, JM#26)
d-Ac-SLRWSRKMPCVS
(SEQ ID NO.: 30, JM#28)
d-MLRWSRKMPCVS
(SEQ ID NO.: 31, JM#29)
d-LLRWSRKMPCVS
(SEQ ID NO.: 32, JM#30)
d-FLRWSRKMPCVS
(SEQ ID NO.: 33, JM#31)
GLRWSRKMPCVS
(SEQ ID NO.: 34, JM#32)
Ac-SMRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 39, JM#37)
d-FMRWSRKMPCVS
(SEQ ID NO.: 40, JM#38)
d-GMRWSRKMPCVS
(SEQ ID NO.: 42, JM#40)
d-MLRWSRKLPCVS
(SEQ ID NO.: 43, JM#41)
Id-LRWSRKLPCVS
(SEQ ID NO.: 44, JM#42)
Id-LRWSRKMPCVS
(SEQ ID NO.: 45, JM#43)
Md-LRWSRKLPCVS
(SEQ ID NO.: 46, JM#44)
Md-LRWSRKMPCVS
(SEQ ID NO.: 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS

Group 6 consists of

(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS

Group 7 consists of

(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 70, JM#173)
d-ILRWSRK-NH2
(SEQ ID NO.: 71, JM#174)
d-ILRWSRKLP-NH2

Group 8 consists of

(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2

Group 9 consists of

(SEQ ID NO.: 27, JM#25)
Ac-SLRWSRKMPCVS
(SEQ ID NO.: 30, JM#28)
d-MLRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 52, JM#140)
d-LLRWSRK(Glu-Pal)MPCVS
(SEQ ID NO.: 75, JM#178)
d-LMRWSRK(Pal)MP-NH2

Group 10 consists of

(SEQ ID NO.: 55, JM#144)
ILRWSRK(Pal)LPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 69, JM#172)
ILRWSRK(Pal)L-NH2
(SEQ ID NO.: 77, JM#180)
d-LMRWSRK(Pal)-NH2
(SEQ ID NO.: 88, JM#191)
ILRWSRKK(Glu-Pal)-NH2
(SEQ ID NO.: 89, JM#192)
ILRWSRK(Glu-Pal)-NH2
(SEQ ID NO.: 90, JM#193)
ILRWSRKLK(Glu-Pal)-NH2
(SEQ ID NO.: 91, JM#194)
d-LMRWSRK(Glu-Pal)-NH2

wherein d- in front of an amino acid indicates a D-Amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol, and Ac indicates a substitution of an amino group by an acetyl group, wherein the peptide is coupled to a polymer.
4. The peptide according to clause 3, wherein the peptide is coupled to PEG.
5. The peptide according to clause 4, wherein the peptide is coupled to 1,2-distearoyl-sn-glycero-3-phosphoethanolamine.
6. The peptide according to clause 3, wherein the peptide is coupled to a poly(vinyl alcohol).
7 The peptide according to clause 3, wherein the peptide is a poly(vinyl pyrrolidone).
8. A peptide consisting of two identical monomeric peptides according to any of the preceding clauses, wherein the monomeric peptides are linked to each other via a cysteine bridge which is formed between the monomeric peptides to form a dimeric peptide.
9. The peptide or the pharmaceutical composition according to any of the preceding clauses for use in medicine.
10. Pharmaceutical composition comprising the peptide according to any of the preceding clauses, together with at least one pharmaceutically acceptable carrier, mesoporous nanoparticles, cryoprotectant, lyoprotectant, excipient and/or diluent.
11. Use of the peptide or the pharmaceutical composition according to any of the preceding clauses for the preparation of a formulation for oral administration, inhalation, intravenous administration, topical administration, intranasal, intraperitoneal administration, subcutaneous administration and/or any other injectable form.
12. Use according to clause 11, wherein the peptide or the pharmaceutical composition are used for the preparation of a lyophilized formulation of a buffered liquid formulation.
13. The peptide or the pharmaceutical composition according to any of the preceding clauses for use in theranostics, in the treatment of disorders of hematopoiesis, in particular for support of the mobilization, proliferation and migration of stem cells; in the treatment of wounds, in particular wounds caused by burning; in the treatment of viral diseases, in particular infections with HIV-1, HIV-2, Cytomegalovirus, Herpes simplex virus (type 1 and 2), Varicella zoster virus, Hepatitis A and Hepatitis B virus, Influenza virus, Polio virus, Rhino virus, Rubella virus, Measles virus, Rabies virus, Rous sarcoma virus, Epstein-Barr Virus; in the treatment of infections caused by bacteria and fungi, in particular Pseudomonas, Candida, S. aureus; in the treatment of infectious processes, abnormal infectious processes; in the treatment of inflammation, in particular of periodontal disease; in the treatment of growth disorders; in the treatment of neuronal diseases, disorders of the blood clotting cascade and hematopoiesis, vascular diseases, diseases of the immune system, for improving wound and bone healing, for use in the treatment of neurological diseases, in particular stroke, Parkinson's disease, Alzheimer's disease, multiple sclerosis; in the treatment of warts, Hypogammaglobulinemia, Immunodeficiency, and Myelokathexis syndrome and rheumatoid arthritis; in the treatment of cancers, in particular cancers showing the CXCR receptor, preferably cancer of the liver, pancreas, prostate, breast cancer or other solid tumors; in the treatment of lack of mobilization, proliferation and migration of stem cells, T-cell activation as well as support of immunoblasts, preferably of cytotoxic T lymphocytes with programmed cell death receptor 1; in the treatment of antifibrosis; in the treatment or prevention of scars; in the treatment of cardiologic disorders, in particular heart insufficiency; in the treatment of metabolic disorders, in particular diabetes.
14. A method of prophylaxis and/or treatment of cancer, viral diseases, metabolic disorders, neurologic disorders, diseases of the immune system, and disorders of the blood clotting cascade and hematopoiesis in a mammal, including a human.
15. A method for manufacturing the peptide according to any of the preceding clauses by solid phase synthesis.

### REFERENCES

Hendrix C W, Flexner C, MacFarland R T, Giandomenico C, Fuchs E J, Redpath E, Bridger G, Henson G W, 2000, Antimicrob Agents Chemother 44: 1667-1673.
Münch J, Rajan D, Schindler M, Specht A, Rücker E, Novembre, F J, Nerrienet, E, Müller-Trutwin, M C, Peeters, M, Hahn, B H and Kirchhoff, F, 2007, J Virol 81: 13852-13864.
Balabanian K, Levoye A, Klemm L, Lagane B, Hermine O, Harriague J, Baleux F, Arenzana-Seisdedos F, Bachelerie F, 2008 J Clin Invest 118: 1074-1084.

1. A peptide consisting of one of the following amino acid sequences, selected from any of the groups 1 to 11, wherein
Group 7 consists of
(SEQ ID NO.: 70, JM#173)
d-ILRWSRK-NH2
(SEQ ID NO.: 45, JM#43)
Md-LRWSRKLPCVS
(SEQ ID NO.: 46, JM#44)
Md-LRWSRKMPCVS
(SEQ ID NO.: 54, JM#143)
ILRWSRK(Glu-Pal)LPCVS
(SEQ ID NO.: 55, JM#144)
ILRWSRK(Pal)LPCVS
(SEQ ID NO.: 56, JM#145)
ILRWSRKLPCK(Glu-Pal)S
(SEQ ID NO.: 59, JM#149)
ILRWSRK(Pal)MPCLS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 71, JM#174)
d-ILRWSRKLP-NH2
(SEQ ID NO.: 95, JM#198)
ILRWSRK(Glu-Ste)LPCVS
(SEQ ID NO.: 104, JM#213)
ILRWSRK(Glu-Lau)LPCVS
(SEQ ID NO.: 108, JM#217)
ILRWSRK(Glu-Ole)LPCVS
(SEQ ID NO.: 117, JM#226)
ILRWSRK(C16diacid)LPCVS
(SEQ ID NO.: 118, JM#227)
ILRWSRK(Glu-C16diacid)LPCVS
(SEQ ID NO.: 119, JM#228)
ILRWSRK(C18diacid)LPCVS
(SEQ ID NO.: 120, JM#229)
ILRWSRK(Glu-C18diacid)LPCVS
(SEQ ID NO.: 121, JM#230)
ILRWSRK(OEG-OEG-γGlu-C18diacid)LPCVS
(SEQ ID NO.: 130, JM#235)
d-LLRWSRK(Glu-Pal)-NH2
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 133, JM#238)
d-ILRWSRK(Glu-Pal)-NH2
(SEQ ID NO.: 139, JM#244)
ILRWSRK(C16diacid)-NH2
(SEQ ID NO.: 140, JM#+245)
ILRWSRK(C18diacid)-NH2
(SEQ ID NO.: 141, JM#246)
ILRWSRK(Glu-C16diacid)-NH2
(SEQ ID NO.: 142, JM#247)
ILRWSRK(Glu-C18diacid)-NH2
(SEQ ID NO.: 143, JM#248)
d-LLRWSRK(C16diacid)-NH2
(SEQ ID NO.: 144, JM#249)
d-LLRWSRK(C18diacid)-NH2
(SEQ ID NO.: 145, JM#250)
d-LLRWSRK(Glu-C16diacid)-NH2
(SEQ ID NO.: 146, JM#251)
d-LLRWSRK(Glu-C18diacid)-NH2
(SEQ ID NO.: 147, JM#252)
ILRWSRK(Ara)LPCVS
(SEQ ID NO.: 148, JM#253)
ILRWSRK(Glu-Ara)LPCVS
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 151, JM#256)
d-LLRWSRK(Ste)-NH2
(SEQ ID NO.: 152, JM#257)
d-LLRWSRK(Glu-Ste)-NH2
(SEQ ID NO.: 157, JM#260)
d-LLRWSRK(Glu-Myr)-NH2
(SEQ ID NO.: 159, JM#262)
ILRWSRK(Myr)-NH2
(SEQ ID NO.: 161, JM#264)
LVRYTKK(Glu-Pal)-NH2
(SEQ ID NO.: 162, JM#265)
d-LVRYTKK(Glu-Pal)-NH2
(SEQ ID NO.: 163)
ILRWSRK(Pal-Glu)LPSVS
Group 1 consists of
(SEQ ID NO.: 20, JM#18)
ILRWSRKMPCLS
(SEQ ID NO.: 55, JM#144)
ILRWSRK(Pal)LPCVS
(SEQ ID NO.: 89, JM#192)
ILRWSRK(Glu-Pal)-NH2
(SEQ ID NO.: 91, JM#194)
d-LMRWSRK(Glu-Pal)-NH2
(SEQ ID NO.: 150, JM#255)
ILRWSRK(Glu-Ste)-NH2
Group 2 consists of
(SEQ ID NO.: 69, JM#172)
ILRWSRK(Pal)L-NH2
(SEQ ID NO.: 75, JM#178)
d-LMRWSRK(Pal)MP-NH2
(SEQ ID NO.: 77, JM#180)
d-LMRWSRK(Pal)-NH2
(SEQ ID NO.: 80, JM#183)
ILRWSRK(Ole)LPCVS
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 152, JM#257)
d-LLRWSRK(Glu-Ste)-NH2
Group 3 consists of
(SEQ ID NO.: 53, JM#141)
d-LLRWSRK(Pal)MPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 66, JM#169)
IVRWSKK(Pal)VPCVS
(SEQ ID NO.: 67, JM#170)
ILRWSRK(Pal)-NH2
(SEQ ID NO.: 68, JM#171)
ILRWSRK(Pal)LP-NH2
(SEQ ID NO.: 88, JM#191)
ILRWSRKK(Glu-Pal)-NH2
(SEQ ID NO.: 90, JM#193)
ILRWSRKLK(Glu-Pal)-NH2
(SEQ ID NO.: 96, JM#199)
ILRWSRKK(Glu-Ste)LPCVS
(SEQ ID NO.: 98, JM#203)
ILRWSRK(Glu-Dec)LPCVS
(SEQ ID NO.: 100, JM#205)
ILRWSRK(Glu-Ste)LPCVS
(SEQ ID NO.: 107, JM#216)
ILRWSRK(Myr)LPCVS
(SEQ ID NO.: 149, JM#254)
ILRWSRK(Ste)-NH2
(SEQ ID NO.: 157, JM#260)
d-LLRWSRK(Glu-Myr)-NH2
(SEQ ID NO.: 160, JM#263)
ILRWSRK(Glu-Myr)-NH2
Group 4 consists of
(SEQ ID NO.: 10, JM#8)
ILRWSRKVPCVS
(SEQ ID NO.: 12, JM#10)
IFRWSRKVPCVS
(SEQ ID NO.: 29, JM#27)
MLRWSRKMPCVS
(SEQ ID NO.: 36, JM#34)
MMRWSRKMPCVS
(SEQ ID NO.: 41, JM#39)
MLRWSRKLPCVS
(SEQ ID NO.: 51, JM#122)
ILRWSRKLPSVS
(SEQ ID NO.: 52, JM#140)
d-LLRWSRK(Glu-Pal)MPCVS
(SEQ ID NO.: 59, JM#149)
ILRWSRK(Pal)MPCLS
(SEQ ID NO.: 92, JM#195)
d-LMRWSRKK(Glu-Pal)-NH2
(SEQ ID NO.: 97, JM#200)
ILRWSRK-AcLPCVS
(SEQ ID NO.: 105, JM#214)
ILRWSRK(Lau)LPCVS
(SEQ ID NO: 151, JM#256)
d-LLRWSRK(Ste)-NH2
(SEQ ID NO.: 159, JM#262)
ILRWSRK(Myr)-NH2
Group 5 consists of
(SEQ ID NO.: 3, JM#1)
ILRWSKKVPCVS
(SEQ ID NO.: 4, JM#2)
IFRWSKKVPCVS
(SEQ ID NO.: 5, JM#3)
IVRWSRKVPCVS
(SEQ ID NO.: 6, JM#4)
IVRWSHKVPCVS
(SEQ ID NO.: 7, JM#5)
IVRWSKKLPCVS
(SEQ ID NO.: 8, JM#6)
IVRWSKKIPCVS
(SEQ ID NO.: 9, JM#7)
IVRWSKKFPCVS
(SEQ ID NO.: 11, JM#9)
ILRWSHKVPCVS
(SEQ ID NO.: 13, JM#11)
IFRWSHKVPCVS
(SEQ ID NO.: 14, JM#12)
IVRWSKKMPCVS
(SEQ ID NO.: 16, JM#14)
IVRWSKKVPCd-VS
(SEQ ID NO.: 17, JM#15)
ILRWSRKVPCd-VS
(SEQ ID NO.: 18, JM#16)
IIRWSRKMPCVS
(SEQ ID NO.: 25, JM#23)
ILRWSRKVPSVS
(SEQ ID NO.: 26, JM#24)
ILRWSRKMPSVS
(SEQ ID NO.: 27, JM#25)
Ac-SLRWSRKMPCVS
(SEQ ID NO.: 28, JM#26)
d-Ac-SLRWSRKMPCVS
(SEQ ID NO.: 30, JM#28)
d-MLRWSRKMPCVS
(SEQ ID NO.: 31, JM#29)
d-LLRWSRKMPCVS
(SEQ ID NO.: 32, JM#30)
d-FLRWSRKMPCVS
(SEQ ID NO.: 33, JM#31)
GLRWSRKMPCVS
(SEQ ID NO.: 34, JM#32)
Ac-SMRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 39, JM#37)
d-FMRWSRKMPCVS
(SEQ ID NO.: 40, JM#38)
d-GMRWSRKMPCVS
(SEQ ID NO.: 42, JM#40)
d-MLRWSRKLPCVS
(SEQ ID NO.: 43, JM#41)
Id-LRWSRKLPCVS
(SEQ ID NO.: 44, JM#42)
Id-LRWSRKMPCVS
(SEQ ID NO.: 45, JM#43)
Md-LRWSRKLPCVS
(SEQ ID NO.: 46, JM#44)
Md-LRWSRKMPCVS
(SEQ ID NO.: 47, JM#106)
IVRWSKKVP-NH2
(SEQ ID NO.: 48, JM#110)
IVRWSKK-NH2
(SEQ ID NO.: 49, JM#114)
ILRWSRKLP-NH2
(SEQ ID NO.: 50, JM#118)
ILRWSRK-NH2
(SEQ ID NO.: 54, JM#143)
ILRWSRK(Glu-Pal)LPCVS
(SEQ ID NO.: 56, JM#145)
ILRWSRKLPCK(Glu-Pal)S
(SEQ ID NO.: 57, JM#146)
IYRWSRKMPCLS
(SEQ ID NO.: 58, JM#148)
ILRWSRK(Glu-Pal)MPCLS
(SEQ ID NO.: 60, JM#151)
IVRWSKKVPSVS
(SEQ ID NO.: 61, JM#164)
IVRWSK(Pal)K-NH2
(SEQ ID NO.: 62, JM#165)
IVRWSKK(Pal)-NH2
(SEQ ID NO.: 65, JM#168)
IVRWSK(Pal)KVPCVS
(SEQ ID NO.: 70, JM#173)
d-ILRWSRK-NH2
(SEQ ID NO.: 71, JM#174)
d-ILRWSRKLP-NH2
(SEQ ID NO.: 72, JM#175)
d-ILRWSRK(Pal)LP-NH2
(SEQ ID NO.: 73, JM#176)
d-LMRWSRK(Pal)MPCVS
(SEQ ID NO.: 74, JM#177)
Md-LRWSRK(Pal)LPCVS
(SEQ ID NO.: 76, JM#179)
Md-LRWSRK(Pal)LP-NH2
(SEQ ID NO.: 78, JM#181)
Md-LRWSRK(Pal)-NH2
(SEQ ID NO.: 79, JM#182)
ILRWSRK(Ste)LPCVS
(SEQ ID NO.: 81, JM#184)
ILRWSRK(Chl)LPCVS
(SEQ ID NO.: 82, JM#185)
Ac-ILRWSRKLPCVS
(SEQ ID NO.: 83, JM#186)
d-Ac-ILRWSRKLPCVS
(SEQ ID NO.: 84, JM#187)
Ac-MLRWSRKLPCVS
(SEQ ID NO.: 85, JM#188)
d-Ac-MLRWSRKLPCVS
(SEQ ID NO.: 86, JM#189)
VLRWSRKLPCVS
(SEQ ID NO.: 87, JM#190)
d-VLRWSRKLPCVS
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 99, JM#204)
ILRWSRK(Glu-Myr)LPCVS
(SEQ ID NO.: 106, JM#215)
ILRWSRK(Dec)LPCVS
(SEQ ID NO.: 131, JM#236)
d-LLRWSRK(Pal)-NH2
(SEQ ID NO.: 132, JM#237)
d-ILRWSRK(Pal)-NH2
(SEQ ID NO.: 158, JM#261)
d-LLRWSRK(Myr)-NH2
(SEQ ID NO.: 163)
ILRWSRK(Pal-Glu)LPSVS
(SEQ ID NO.: 164)
ILRWSRK(Glu-Ste)LPSVS
Group 6 consists of
(SEQ ID NO.: 92, JM#195)
d-LMRWSRKK(Glu-Pal)-NH2
(SEQ ID NO.: 93, JM#196)
d-MLRWSRK(Glu-Pal)-NH2
(SEQ ID NO.: 94, JM#197)
d-MLRWSRKK(Glu-Pal)-NH2
(SEQ ID NO.: 95, JM#198)
ILRWSRK(Glu-Ste)LPCVS
(SEQ ID NO.: 164)
ILRWSRK(Glu-Ste)LPSVS
Group 8 consists of
(SEQ ID NO.: 31, JM#29)
d-LLRWSRKMPCVS
(SEQ ID NO.: 69, JM#172)
ILRWSRK(Pal)L-NH2
(SEQ ID NO.: 89, JM#192)
ILRWSRK(Glu-Pal)-NH2
Group 9 consists of
(SEQ ID NO.: 27, JM#25)
Ac-SLRWSRKMPCVS
(SEQ ID NO.: 30, JM#28)
d-MLRWSRKMPCVS
(SEQ ID NO.: 35, JM#33)
d-Ac-SMRWSRKMPCVS
(SEQ ID NO.: 37, JM#35)
d-MMRWSRKMPCVS
(SEQ ID NO.: 38, JM#36)
d-LMRWSRKMPCVS
(SEQ ID NO.: 52, JM#140)
d-LLRWSRK(Glu-Pal)MPCVS
(SEQ ID NO.: 75, JM#178)
d-LMRWSRK(Pal)MP-NH2
(SEQ ID NO.: 106, JM#215)
ILRWSRK(Dec)LPCVS
(SEQ ID NO.: 158, JM#261)
d-LLRWSRK(Myr)-NH2
(SEQ ID NO.: 160, JM#263)
ILRWSRK(Glu-Myr)-NH2
Group 10 consists of
(SEQ ID NO.: 55, JM#144)
ILRWSRK(Pal)LPCVS
(SEQ ID NO.: 63, JM#166)
IVRWSK(Pal)KVP-NH2
(SEQ ID NO.: 64, JM#167)
IVRWSKK(Pal)VP-NH2
(SEQ ID NO.: 67, JM#170)
ILRWSRK(Pal)-NH2
(SEQ ID NO.: 68, JM#171)
ILRWSRK(Pal)LP-NH2
(SEQ ID NO.: 69, JM#172)
ILRWSRK(Pal)L-NH2
(SEQ ID NO.: 77, JM#180)
d-LMRWSRK(Pal)-NH2
(SEQ ID NO.: 88, JM#191)
ILRWSRKK(Glu-Pal)-NH2
(SEQ ID NO.: 89, JM#192)
ILRWSRK(Glu-Pal)-NH2
(SEQ ID NO.: 90, JM#193)
ILRWSRKLK(Glu-Pal)-NH2
(SEQ ID NO.: 91, JM#194)
d-LMRWSRK(Glu-Pal)-NH2
(SEQ ID NO.: 104, JM#213)
ILRWSRK(Glu-Lau)LPCVS
(SEQ ID NO.: 130, JM#235)
d-LLRWSRK(Glu-Pal)-NH2
(SEQ ID NO.: 133, JM#238)
d-ILRWSRK(Glu-Pal)-NH2
(SEQ ID NO.: 149, JM#254)
ILRWSRK(Ste)-NH2
(SEQ ID NO.: 150, JM#255)
ILRWSRK(Glu-Ste)-NH2
(SEQ ID NO.: 152, JM#+257)
d-LLRWSRK(Glu-Ste)-NH2
(SEQ ID NO 157, JM#260)
d-LLRWSRK(Glu-Myr)-NH2
Group 11 consists of
ILRWCRKPC-NH2, wherein the cysteine at position 5 is bound to the cysteine at position 9 via a disulfide bond to form a cyclic peptide (SEQ ID NO.: 109, JM #218)
ILRW(d-C)RKPC-NH2, wherein the d-cysteine at position 5 is bound to the cysteine at position 9 via a disulfide bond to form a cyclic peptide (SEQ ID NO.: 111, JM #219)
IPRW(d-C)RKC-NH2, wherein the d-cysteine at position 5 is bound to the cysteine at position 8 via a disulfide bond to form a cyclic peptide (SEQ ID NO.: 113, JM #220)
ILRWSRKLPCVS, wherein the lysine at position 7 is bound to the carboxyl-terminus via a peptide bond to form a cyclic peptide (SEQ ID NO.: 115, JM #221)
ILRWSKKLPCVS, wherein the lysine at position 6 is bound to the carboxyl-terminus via a peptide bond to form a cyclic peptide (SEQ ID NO.: 116, JM #222)
IPRW(d-C)RKP, wherein the d-cysteine at position 5 is bound to the proline at position 8 via a thioester bond to form a cyclic peptide (SEQ ID NO.: 122, JM #231)
ILRW(d-C)RKP, wherein the d-cysteine at position 5 is bound to the proline at position 8 via a thioester bond to form a cyclic peptide (SEQ ID NO.: 124, JM #232)
IPRW(d-S)RKP, wherein the d-serine at position 5 is bound to the proline at position 8 via an ester bond to form a cyclic peptide (SEQ ID NO.: 126, JM #233)
ILRW(d-S)RKP, wherein the d-serine at position 5 is bound to the proline at position 8 via an ester bond to form a cyclic peptide (SEQ ID NO.: 128, JM #234)
IMRWCRKPC-NH12, wherein the cysteine at position 5 is bound to the cysteine at position 9 via a disulfide bond to form a cyclic peptide (SEQ ID NO.: 153, JM #258)
IPRW(d-C)RKCP-NH2, wherein the d-cysteine at position 5 is bound to the cysteine at position 8 via a disulfide bond to form a cyclic peptide (SEQ ID NO.: 155, JM #259),
wherein d- in front of an amino acid indicates a D-amino acid, Pal indicates a palmitic acid on the preceding amino acid, Glu-Pal indicates a palmitic acid on the preceding amino acid with a glutamate linker, Dec indicates decanoic acid on the preceding amino acid, Glu-Dec indicates a decanoic acid on the preceding amino acid with a glutamate linker, Myr indicates myristic acid on the preceding amino acid, Glu-Myr indicates a myristic acid on the preceding amino acid with a glutamate linker, Ole indicates oleic acid on the preceding amino acid, Ste indicates stearic acid on the preceding amino acid, Glu-Ste indicates a stearic acid on the preceding amino acid with a glutamate linker, Chl indicates Cholesterol on the preceding amino acid, Ac indicates a substitution of an amino group by an acetyl group, Lau indicates lauric acid on the preceding amino acid, Glu-Lau indicates a lauric acid on the preceding amino acid with a glutamate linker, Glu-Ole indicates an oleic acid on the preceding amino acid with a glutamate linker, C16diacid indicates a saturated C16 fatty diacid on the preceding amino acid, Glu-C16diacid indicates a saturated C16 fatty diacid on the preceding amino acid with a glutamate linker, C18diacid indicates a saturated C18 fatty diacid on the preceding amino acid, Glu-C18diacid indicates a saturated C18 fatty diacid on the preceding amino acid with a glutamate linker, OEG-OEG-γGlu-C18diacid indicates a saturated C18 fatty diacid on the preceding amino acid with an OEG-OEG-gamma glutamate linker, OEG represents the residue of 8-amino-3,6-dioxaoctanoic acid, Ara indicates a saturated C20 fatty acid on the preceding amino acid, and Glu-Ara indicates a saturated C20 fatty acid on the preceding amino acid with a glutamate linker.

2-9. (canceled)

10. A peptide consisting of two identical monomeric peptides according to claim 1, wherein the monomeric peptides are linked to each other via a cysteine bridge which is formed between the monomeric peptides to form a dimeric peptide.

11. Pharmaceutical composition comprising the peptide according to claim 1, together with at least one pharmaceutically acceptable carrier, mesoporous nanoparticles, cryoprotectant, lyoprotectant, excipient and/or diluent.

12. (canceled)

13. A method of treating a CXCR4-related medical condition, comprising administering a peptide that binds to CXCR4, wherein the peptide that binds to CXCR4 is a peptide according to claim 1 to a mammal in need thereof.

14. The method LT-se according to claim 13, wherein the peptide is formulated as a lyophilized formulation of a buffered liquid formulation.

15. (canceled)

16. The method according to claim 13, wherein the peptide is administered by oral administration, inhalation, intravenous administration, topical administration, intranasal administration, intraperitoneal administration, subcutaneous administration, and/or any other injectable form.

17. The method according to claim 13, wherein the disease or condition associated with CXCR4 is cancer, a viral disease, an infection caused by bacteria and fungi, a metabolic disorder, a neurologic disorder, a disease of the immune system, inflammation, a growth disorder, a disorder of the blood clotting cascade or hematopoiesis, a vascular disease, a wound, fibrosis, a cardiologic disorder, a metabolic disorder, or a lung disease.

18. A conjugate comprising a peptide according to claim 1 coupled to a second agent.

19. The conjugate of claim 18, wherein the second agent is a complexing agent.

20. The conjugate of claim 19, wherein the complexing agent is dodecane tetraacetic acid (DOTA) or deferoxamine.

21. The conjugate according to claim 20, wherein the peptide is labelled with a radioactive nuclide.

22. The conjugate of claim 18, wherein the second agent is a polymer.

23. The conjugate of claim 18, wherein the second agent is PEG.

24. The conjugate of claim 18, wherein the second agent is 1,2-distearoyl-sn-glycero-3-phosphoethanolamine.

25. The conjugate of claim 18, wherein the second agent is a poly(vinyl alcohol).

26. The conjugate of claim 18, wherein the second agent is a poly(vinyl pyrrolidone).

27. The conjugate of claim 18, wherein the second agent is a further peptide.

28. The conjugate of claim 27, wherein the further peptide is a copy of the peptide according to claim 1.

29. The conjugate of claim 18, wherein the second agent is cholesterol.

30. The conjugate of claim 18, wherein the second agent a drug.

31. The conjugate of claim 18, wherein the second agent human serum albumin.

32. A method for manufacturing the peptide according to claim 1 by solid phase synthesis.
